(19) United States
(12) Patent Application Publication
(10) Pub. No.: US 2009/0239214 A1
(43) Pub. Date: Sep. 24, 2009

(54) Prognosis of breast cancer patients

(76) Inventors: Hongyue Dai, Kenmore, WA (US); Yudong He, Kirkland, WA (US); Mao Mao, Redmond, WA (US); Bernard Fine, Menlo Park, CA (US); Laura J. van't Veer, Amsterdam (NL); Marc J. van de Vijver, Amsterdam (NL)

Correspondence Address: JONES DAY, 222 EAST 41ST ST, NEW YORK, NY, 10017, US

(21) Appl. No.: 11/658,605

(22) PCT Filed: Aug. 1, 2005
(86) PCT No.: PCT/US05/27243

§ 371 Date: Feb. 27, 2009

Related U.S. Application Data
(60) Provisional application No. 60/592,858, filed on Jul. 30, 2004.

(51) Int. Cl. C12Q 1/68; G06F 19/00

(52) U.S. Cl. 435/6; 702/19

(57) ABSTRACT

The present invention relates to sets of genetic markers whose expression is correlated with prognosis of breast cancer in individuals having breast cancer. Specifically, the invention provides sets of markers whose expression patterns can be used to differentiate individuals having a good prognosis, e.g., no reoccurrence or metastasis within five years of initial diagnosis, and individuals having a poor prognosis, e.g., reoccurrence or metastasis within five years of initial diagnosis. The invention relates to methods of prognosis using these markers. The invention also relates to microarrays containing probes to these markers, and to kits containing ready-to-use microarrays and computer software for data analysis using the prognostic and statistical methods disclosed herein.

This application claims the benefit under 35 U.S.C. § 119(e) of U.S. Provisional Patent Application No. 60/592,858, filed on Jul. 30, 2004, which is incorporated by reference herein in its entirety.

### 1. FIELD OF THE INVENTION

The present invention relates to the identification of marker genes useful in the diagnosis and prognosis of breast cancer. More particularly, the invention relates to the identification of sets of marker genes able to distinguish individuals having breast cancer with good clinical prognosis from individuals with poor clinical prognosis. The invention further relates to methods of distinguishing breast cancer-related conditions using the identified sets of markers. The invention further provides methods for determining the course of treatment of a patient with breast cancer.

### 2. BACKGROUND OF THE INVENTION

The increased number of cancer cases reported in the United States, and, indeed, around the world, is a major concern. Currently there are only a handful of treatments available for specific types of cancer, and these provide no guarantee of success. In order to be most effective, these treatments require not only an early detection of the malignancy, but a reliable assessment of the severity of the malignancy.

The incidence of breast cancer, a leading cause of death in women, has been gradually increasing in the United States over the last thirty years. Its cumulative risk is relatively high; 1 in 8 women are expected to develop some type of breast cancer by age 85 in the United States. In fact, breast cancer is the most common cancer in women and the second most common cause of cancer death in the United States. In 1997, it was estimated that 181,000 new cases were reported in the U.S., and that 44,000 people would die of breast cancer (Parker et al., CA Cancer J. Clin. 47:5-27 (1997); Chu et al., J. Nat. Cancer Inst. 88:1571-1579 (1996)). While mechanism of tumorigenesis for most breast carcinomas is largely unknown, there are genetic factors that can predispose some women to developing breast cancer (Miki et al., Science, 266:66-71 (1994)). The discovery and characterization of BRCA1 and BRCA2 has recently expanded our knowledge of genetic factors which can contribute to familial breast cancer. Germ-line mutations within these two loci are associated with a 50 to 85% lifetime risk of breast and/or ovarian cancer (Casey, Curr. Opin. Oncol. 9:88-93 (1997); Marcus et al., Cancer 77:697-709 (1996)). Only about 5% to 10% of breast cancers are associated with breast cancer susceptibility genes, BRCA1 and BRCA2. The cumulative lifetime risk of breast cancer for women who carry the mutant BRCA1 is predicted to be approximately 92%, while the cumulative lifetime risk for the non-carrier majority is estimated to be approximately 10%. BRCA1 is a tumor suppressor gene that is involved in DNA repair and cell cycle control, which are both important for the maintenance of genomic stability. More than 90% of all mutations reported so far result in a premature truncation of the protein product with abnormal or abolished function. The histology of breast cancer in BRCA1 mutation carriers differs from that in sporadic cases, but mutation analysis is the only way to find the carrier. Like BRCA1, BRCA2 is involved in the development of breast cancer, and like BRCA1 plays a role in DNA repair. However, unlike BRCA1, it is not involved in ovarian cancer.

Other genes have been linked to breast cancer, for example c-erb-2 (HER2) and p53 (Beenken et al., Ann. Surg. 233(5):630-638 (2001). Overexpression of c-erb-2 (HER2) and p53 have been correlated with poor prognosis (Rudolph et al., Hum. Pathol. 32(3):311-319 (2001), as has been aberrant expression products of mdm2 (Lukas et al., Cancer Res. 61(7):3212-3219 (2001) and cyclin1 and p27 (Porter & Roberts, International Publication WO98/33450, published Aug. 6, 1998). However, no other clinically useful markers consistently associated with breast cancer have been identified.

Sporadic tumors, those not currently associated with a known germline mutation, constitute the majority of breast cancers. It is also likely that other, non-genetic factors also have a significant effect on the etiology of the disease. Regardless of the cancer's origin, breast cancer morbidity and mortality increases significantly if it is not detected early in its progression. Thus, considerable effort has focused on the early detection of cellular transformation and tumor formation in breast tissue.

A marker-based approach to tumor identification and characterization promises improved diagnostic and prognostic reliability. Typically, the diagnosis of breast cancer requires histopathological proof of the presence of the tumor. In addition to diagnosis, histopathological examinations also provide information about prognosis and selection of treatment regimens. Prognosis may also be established based upon clinical parameters such as tumor size, tumor grade, the age of the patient, and lymph node metastasis.

Diagnosis and/or prognosis may be determined to varying degrees of effectiveness by direct examination of the outside of the breast, or through mammography or other X-ray imaging methods (Jatoi, Am. J. Surg. 177:518-524 (1999)). The latter approach is not without considerable cost, however. Every time a mammogram is taken, the patient incurs a small risk of having a breast tumor induced by the ionizing properties of the radiation used during the test. In addition, the process is expensive and the subjective interpretations of a technician can lead to imprecision. For example, one study showed major clinical disagreements for about one-third of a set of mammograms that were interpreted individually by a surveyed group of radiologists. Moreover, many women find that undergoing a mammogram is a painful experience. Accordingly, the National Cancer Institute has not recommended mammograms for women under fifty years of age, since this group is not as likely to develop breast cancers as are older women. It is compelling to note, however, that while only about 22% of breast cancers occur in women under fifty, data suggests that breast cancer is more aggressive in pre-menopausal women.

In clinical practice, accurate diagnosis of various subtypes of breast cancer is important because treatment options, prognosis, and the likelihood of therapeutic response all vary broadly depending on the diagnosis. Accurate prognosis, or determination of distant metastasis-free survival could allow the oncologist to tailor the administration of adjuvant chemotherapy, with women having poorer prognoses being given the most aggressive treatment. Furthermore, accurate prediction of poor prognosis would greatly impact clinical trials for new breast cancer therapies, because potential study patients could then be stratified according to prognosis. Trials could then be limited to patients having poor prognosis, in turn making it easier to discern if an experimental therapy is efficacious.

Adjuvant systemic therapy has been shown to substantially improve the disease-free and overall survival in both premenopausal and postmenopausal women up to age 70 with lymph node negative and lymph node positive breast cancer. See Early Breast Cancer Trialists' Collaborative Group, Lancet 352(9132):930-942 (1998); Early Breast Cancer Trialists' Collaborative Group, Lancet 351(9114):1451-1467 (1998). The absolute benefit from adjuvant treatment is larger for patients with poor prognostic features and this has resulted in the policy to select only these so-called ‘high-risk’ patients for adjuvant chemotherapy. Goldhirsch et al., Meeting highlights: International Consensus Panel on the Treatment of Primary Breast Cancer, Seventh International Conference on Adjuvant Therapy of Primary Breast Cancer, J. Clin. Oncol. 19(18):3817-3827 (2001); Eifel et al., National Institutes of Health Consensus Development Conference Statement: Adjuvant Therapy for Breast Cancer, Nov. 1-3, 2000, J. Natl. Cancer Inst. 93(13):979-989 (2001). Accepted prognostic and predictive factors in breast cancer include age, tumor size, axillary lymph node status, histological tumor type, pathological grade and hormone receptor status. A large number of other factors has been investigated for their potential to predict disease outcome, but these have in general only limited predictive power. Isaacs et al., Semin. Oncol. 28(1):53-67 (2001).

Using gene expression profiling with cDNA microarrays, Perou et al. showed that there are several subgroups of breast cancer patients based on unsupervised cluster analysis: those of “basal type” and those of “luminal type.” Perou et al., Nature 406(6797):747-752 (2000). These subgroups differ with respect to outcome of disease in patients with locally advanced breast cancer. Sorlie et al., Proc. Natl. Acad. Sci. U.S.A. 98(19): 10869-10874 (2001). In addition, microarray analysis has been used to identify diagnostic categories, e.g., BRCA1 and 2 (Hedenfalk et al., N. Engl. J. Med. 344(8):539-548 (2001); van't Veer et al., Nature 415(6871):530-536 (2002)); estrogen receptor status (Perou, supra; Van't Veer, supra; Gruvberger et al., Cancer. Res. 61(16):5979-5984 (2001)) and lymph node status (West et al., Proc. Natl. Acad. Sci. U.S.A. 98(20):11462-11467 (2001); Ahr et al., Lancet 359(9301):131-132 (2002)). Recently, a collection of 70 markers was identified for breast cancer that could classify an individual as having a good prognosis or poor prognosis. See Van't Veer et al., Nature 415(6871):530-536 (2002). This set of markers was derived from individuals who were all less than 55 years of age.

The power of gene expression analysis in the identification of prognosis-relevant genes having been demonstrated, there still exists a need in the art for additional sets of prognosis-relevant markers for individuals having breast cancer, e.g., individuals 55 years of age and older. The present invention provides marker sets that are useful for the prognosis of breast cancer in individuals, particularly individuals 55 years of age and older.

### 3. SUMMARY OF THE INVENTION

The present invention provides a method for classifying an individual with breast cancer as having a good prognosis or a poor prognosis, wherein said individual is 55 years of age or older, comprising detecting a difference in the expression of a first plurality of genes in a cell sample taken from the individual relative to a control, said first plurality of genes comprising 10 of the genes corresponding to the different markers listed in any of Tables 1-8, wherein “good prognosis” is a desired outcome and “poor prognosis” is an undesired outcome. In a specific embodiment, said plurality comprises 20 of the genes corresponding to the different markers listed in any of Tables 1-8. In a specific embodiment of the method, said plurality comprises 50 of the genes corresponding to the different markers listed in any of Tables 1-8. In another specific embodiment, said plurality comprises each of the genes corresponding to the markers listed in Table 1. In another specific embodiment, said plurality comprises each of the genes corresponding to the markers listed in Table 3. In another specific embodiment, said individual is identified as ER+, and said plurality comprises 10 of the genes corresponding to the markers listed in Table 5. In another specific embodiment, said individual is identified as ER+, and said plurality comprises 50 of the genes corresponding to the markers listed in Table 5. In another specific embodiment, said individual is identified as ER+, and said plurality comprises each of the genes corresponding to the markers listed in Table 5. In another specific embodiment, said individual is identified as ER+, and said plurality comprises 10 of the genes corresponding to the markers listed in Table 7. In another embodiment, said individual is identified as ER+, and said plurality comprises 50 of the genes corresponding to the markers listed in Table 7. In another specific embodiment, said individual is identified as ER+, and said plurality comprises each of the genes corresponding to the markers listed in Table 7. In another specific embodiment, said control comprises nucleic acids derived from a pool of tumors from individual sporadic patients. In another specific embodiment, said good prognosis is the non reoccurrence or metastasis within five years of initial diagnosis, and said poor prognosis is the reoccurrence or metastasis within five years of initial diagnosis. In another specific embodiment, said detecting comprises the steps of: (a) generating a good prognosis template by hybridization of nucleic acids derived from a plurality of good prognosis patients against nucleic acids derived from a pool of tumors from individual patients; (b) generating a poor prognosis template by hybridization of nucleic acids derived from a plurality of poor prognosis patients against nucleic acids derived from said pool of tumors from said plurality of individual patients; (c) hybridizing an nucleic acids derived from and individual sample against said pool; and (d) determining the similarity of marker gene expression in the individual sample to the good prognosis template and the poor prognosis template, wherein if said expression is more similar to the good prognosis template, the sample is classified as having a good prognosis, and if said expression is more similar to the poor prognosis template, the sample is classified as having a poor prognosis.

The invention further provides a method for classifying a sample as derived from an individual having a good prognosis or derived from an individual having a poor prognosis, wherein said individual is 55 years of age or older, by calculating the similarity between the expression of at least 10 of the different markers listed in any of Tables 1-8 in the sample to the expression of the same markers in a good prognosis nucleic acid pool and a poor prognosis nucleic acid pool, comprising the steps of: (a) labeling nucleic acids derived from a sample, with a first fluorophore to obtain a first pool of fluorophore-labeled nucleic acids; (b) labeling with a second fluorophore a first pool of nucleic acids derived from two or more samples from individuals having a good prognosis (good prognosis pool), and a second pool of nucleic acids derived from two or more samples from individuals having a poor prognosis (poor prognosis pool): (c) contacting said first fluorophore-labeled nucleic acid and said first pool of second fluorophore-labeled nucleic acid with a first microarray under conditions such that hybridization can occur, and contacting said first fluorophore-labeled nucleic acid and said second pool of second fluorophore-labeled nucleic acid with a second microarray under conditions such that hybridization can occur, wherein said first microarray and said second microarray are similar to each other, exact replicas of each other, or are identical, detecting at each of a plurality of discrete loci on the first microarray a first flourescent emission signal from said first fluorophore-labeled nucleic acid and a second fluorescent emission signal from said first pool of second fluorophore-labeled genetic matter that is bound to said first microarray under said conditions, and detecting at each of the marker loci on said second microarray said first fluorescent emission signal from said first fluorophore-labeled nucleic acid and a third fluorescent emission signal from said second pool of second fluorophore-labeled nucleic acid; (d) determining the similarity of the sample to the good prognosis and poor prognosis pools by comparing said first fluorescence emission signals and said second fluorescence emission signals, and said first emission signals and said third fluorescence emission signals; and (e) classifying the sample as derived from an individual having a good prognosis where the first fluorescence emission signals are more similar to said second fluorescence emission signals than to said third fluorescent emission signals, and classifying the sample as derived from an individual having a poor prognosis where the first fluorescence emission signals are more similar to said third fluorescence emission signals than to said second fluorescent emission signals. In a specific embodiment, said similarity is calculated by determining a first sum of the differences of expression levels for each marker between said first fluorophore-labeled nucleic acid and said first pool of second fluorophore-labeled nucleic acid, and a second sum of the differences of expression levels for each marker between said first fluorophore-labeled nucleic acid and said second pool of second fluorophore-labeled nucleic acid, wherein if said first sum is greater than said second sum, the sample is classified as derived from an individual having a poor prognosis, and if said second sum is greater than said first sum, the sample is classified as derived from an individual having a good prognosis.

The invention further provides a method for determining a set of marker genes whose expression is associated with a particular phenotype, comprising the steps of: (a) selecting a phenotype having two or more phenotype categories; (b) identifying a first plurality of genes, wherein the expression of said genes in a first plurality of samples is correlated or anticorrelated with one of the phenotype categories; (c) predicting the phenotype category of each sample in said plurality of samples based on the expression level of each of said plurality of genes across all other samples in said plurality of samples; (d) selecting those samples for which the phenotype category is correctly predicted, to form a second plurality of samples; (e) identifying a second plurality of genes, wherein the expression of said genes in said second plurality of samples is correlated or anticorrelated with one of the phenotype categories; wherein said second plurality of genes is a set of marker genes whose expression is associated with a particular phenotype. In a specific embodiment, said phenotype is breast cancer, and said phenotype categories are good prognosis and poor prognosis. In another specific embodiment, said second plurality of marker genes is validated by: (a) using a statistical method to randomize the association between said second plurality of marker genes and said phenotype category, thereby creating a control correlation coefficient for each marker gene; (b) repeating step (a) one hundred or more times to develop a frequency distribution of said control correlation coefficients for each marker gene; (c) determining the number of marker genes having a control correlation coefficient above a preselected threshold, thereby creating a control marker gene set; and (d) comparing the number of control marker genes so identified to the number of marker genes, wherein if the difference between the number of marker genes and the number of control genes is statistically significant, said set of marker genes is validated. In another specific embodiment, said second plurality of marker genes is optimized by the method comprising: (a) rank-ordering the genes by amplitude of correlation or by significance of the correlation coefficients to create a rank-ordered list, and (b) selecting an arbitrary number n of marker genes from the top of the rank-ordered list. In a more specific embodiment, said set of marker genes is further optimized by the method comprising: (a) calculating an error rate for said arbitrary number n of marker genes; (b) increasing by 1 the number of genes selected from the top of the rank-ordered list; (c) calculating an error rate for said number of genes selected from the top of the rank-ordered list; (d) repeating steps (b) and (c) until said number of genes selected from the top of the rank-ordered list includes all genes included in said rank ordered list, and (e) identifying said number of genes selected from the top of the rank-ordered list for which the error rate is smallest, wherein said set of marker genes is optimized when the error rate is the smallest.

The invention also provides a method for assigning a person to one of a plurality of categories in a clinical trial, comprising determining for each said person the level of expression of at least 10 of the different prognosis markers listed in any of Tables 1-8, determining therefrom whether the person has an expression pattern that correlates with a good prognosis or a poor prognosis, and assigning said person to one category in a clinical trial if said person is determined to have a good prognosis, and a different category if that person is determined to have a poor prognosis.

The invention further provides a microarray comprising a plurality of probes complementary and hybridizable to at least 10 different genes for which markers are listed in any one of Tables 1-8, wherein said plurality of probes is at least 50% of probes on said microarray. In a specific embodiment, said plurality of probes is at least 50% of probes on said microarray. In another specific embodiment, said plurality of probes is at least 70% of probes on said microarray. In another specific embodiment, said plurality of probes is at least 90% of probes on said microarray. In another specific embodiment, said plurality of probes is at least 95% of probes on said microarray. In another specific embodiment, at least 98% of the probes on the microarray are present in any one of Tables 1-6.

The invention also provides a microarray for distinguishing a cell sample from an individual having a good prognosis from a cell sample from an individual having a poor prognosis, wherein said individual is 55 years of age or older, comprising a positionally-addressable array of polynucleotide probes bound to a support, said polynucleotide probes comprising a plurality of polynucleotide probes of different nucleotide sequences, each of said different nucleotide sequences comprising a sequence complementary and hybridizable to a different gene, said plurality consisting of at least 10 of the different genes corresponding to the markers listed in any of Tables 1-8, wherein said plurality of polynucleotide probes is at least 50% of probes on said microarray.

The invention further provides a kit for determining whether a sample is derived from a patient having a good prognosis or a poor prognosis, wherein said patient is 55 years of age or older, comprising at least one microarray comprising probes to at least 10 of the different genes corresponding to the markers listed in any of Tables 1-8, and a computer readable medium having recorded thereon one or more programs for determining the similarity of the level of nucleic acid derived from the markers listed in Table 1-8 in a sample to that in a pool of samples derived from individuals having a good prognosis and a pool of samples derived from individuals having a good prognosis, wherein the one or more programs cause a computer to perform a method comprising computing the aggregate differences in expression of each marker between the sample and the good prognosis pool and the aggregate differences in expression of each marker between the sample and the poor prognosis pool, or a method comprising determining the correlation of expression of the markers in the sample to the expression in the good prognosis and poor prognosis pools.

The invention also provides a method for classifying a breast cancer patient according to prognosis, wherein said patient is 55 years of age or older, comprising: (a) comparing the respective levels of expression of at least 10 different genes for which markers are listed in any of Tables 1-8 in a cell sample taken from said breast cancer patient to respective control levels of expression of said at least 10 genes; and (b) classifying said breast cancer patient according to prognosis based on the similarity between said levels of expression in said cell sample and said control levels. In a specific embodiment, step (b) comprises determining whether said similarity exceeds one or more predetermined threshold values of similarity. In a more specific embodiment, the method further comprises determining prior to step (a) said level of expression of said at least five genes. In another specific embodiment, said control levels are the mean levels of expression of each of said at least five genes in a pool of tumor samples obtained from a plurality of breast cancer patients who have no distant metastasis within five years of initial diagnosis. In another specific embodiment, said control levels comprise the expression levels of said genes in breast cancer patients who have had no distant metastasis within five years of initial diagnosis. In another specific embodiment, wherein said control levels comprise, for each of said at least five genes, mean log intensity values stored on a computer.

The invention further provides a computer program product for classifying a breast cancer patient according to prognosis, said patient being 55 years of age or older, the computer program product for use in conjunction with a computer having a memory and a processor, the computer program product comprising a computer readable storage medium having a computer program encoded thereon, wherein said computer program product can be loaded into the one or more memory units of a computer and causes the one or more processor units of the computer to execute the steps of: (a) receiving a first data structure comprising the respective levels of expression of each of at least 10 different genes for which markers are listed in any of Tables 1-8 in a cell sample taken from said patient; (b) determining the similarity of the level of expression of each of said at least ten genes to respective control levels of expression of said at least five genes to obtain a patient similarity value; (c) comparing said patient similarity value to a selected threshold value of similarity of said respective levels of expression of each of said at least 10 genes to said respective control levels of expression of said at least 10 genes; and (d) classifying said patient as having a first prognosis if said patient similarity value exceeds said threshold similarity value, and a second prognosis if said patient similarity value does not exceed said threshold similarity value. In a specific embodiment, said threshold value of similarity is a value stored in said computer. In another specific embodiment, said control levels of expression of said at least 10 genes are stored in said computer. In another specific embodiment, said computer program, when loaded into memory, further causes said one or more processor units of the computer to execute the steps of receiving a data structure comprising clinical data specific to said breast cancer patient. In another specific embodiment, said respective control levels of expression of said at least 10 genes comprises a set of single-channel mean hybridization intensity values for each of said at least 10 genes, stored on said computer readable storage medium. In a more specific embodiment, said single-channel mean hybridization intensity values are log transformed. In another specific embodiment, said computer program product causes said processing unit to perform said comparing step (c) by calculating the difference between the level of expression of each of said at least five genes in said cell sample taken from said breast cancer patient and said respective control levels of expression of said at least five genes. In another specific embodiment, said computer program product causes said processing unit to perform said comparing step (c) by calculating the mean log level of expression of each of said at least 10 genes in said control to obtain a control mean log expression level for each gene, calculating the log expression level for each of said at least 10 genes in a breast cancer sample from said patient to obtain a patient log expression level, and calculating the difference between the patient log expression level and the control mean log expression for each of said at least 10 genes. In another specific embodiment, said computer program product causes said processing unit to perform said comparing step (c) by calculating similarity between the level of expression of each of said at least 10 genes in said cell sample taken from said patient and said respective control levels of expression of said at least 10 genes, wherein said similarity is expressed as a similarity value. In a more specific embodiment, said similarity value is a correlation coefficient.

### 4. BRIEF DESCRIPTION OF THE FIGURES

FIG. 1. Overview of gene expression data for a sample group of 153 breast cancer tumors from patients of age >55 years over approximately 10,000 significant genes. Each row displays a tumor profile, and each column displays the data for a gene. White indicates the most overexpression relative to the reference pool, black indicates the most underexpression relative to the reference pool, and medium gray indicates no change.

FIG. 2. The predictive power of the 70 marker classifier (see Van't Veer et al., Nature 415(6871):530-536 (2002)) for 153 tumors from this study. The overall odds ratio is 2.5 [26 38 19 70], and 5 year odds ratio is 5.2 [21 30 8 59]. The overall error rate is 0.387 and 5 year error rate is 0.306. Error rates for prediction of outcome for good outcome samples and poor outcome samples were calculated based upon the selected threshold (X-axis). Circles: Error rate for good prognosis samples. Stars: Error rates for poor prognosis samples. Line: average of good prognosis and poor prognosis error rates.

FIG. 3. Expression pattern of 70 prognosis markers identified by a clustering method previously as described (see Van't Veer et al., Nature 415(6871):530-536 (2002)) based on breast tumor profiles from patients of age <55 years. The pattern is associated with the metastasis as shown in the panel on the right (=1 for metastasis samples and =0 for metastasis-free samples).

FIG. 4. Procedures used in identifying the optimal set of discriminating genes for the purpose of prognosis (the “Homogeneous method”, also called “iterative algorithm”).

FIG. 5. The classification error rate for type 1 and type 2 together as a function of the number of discriminating genes used in the classifier. The combined optimal error rate is reached by approximately 200 discriminating marker genes. The classifier was constructed by using the same method used in out previous study (see Van't Veer et al., Nature 415(6871):530-536 (2002)) (“the Nature method”) and as described herein. Circles: error for a particular number of markers used in the classifier. X-axis: number of reporters. Y-axis: error rate.

FIG. 6. Expression profile of 200 reporter genes in the optimal prognosis classifier constructed by the Nature method (Van't Veer (2000)) based on breast tumor profiles from patients with age >55 years. The profile can be used to predict the metastasis status as shown in the panel on right side (1=metastasis within 5 years of initial diagnosis; and 0=metastasis-free at least within 5 years of initial diagnosis).

FIG. 7. The classification error rate for type 1 and type 2 together as a function of the number of discriminating genes used in the classifier. The combined optimal error rate is reached at approximately 100 discriminating marker genes. The classifier is modeled by using the new method discussed in the text (“the homogenous method”). Circles: error for a particular number of markers used in the classifier. X-axis: number of reporters. Y-axis: error rate.

FIGS. 8A, 8B. FIG. 8A: Scattering plots between the correlation of tumor profiles to “poor outcome group” and the correlation of tumor profiles to “good outcome group” based on the new optimal classifier. Filled circles: good outcome patients. Squares: poor outcome patients. FIG. 8B Type 1 error rate, type 2 error rate, and average type 1 and type 2 error rate as a function of threshold.

FIG. 9. Gene expression pattern of 100 genes identified by the “iterative algorithn” (see Example 3) that can be used to predict the disease outcome. The profile can be used to predict the metastasis status as shown in the panel on right side (1=metastasis; 0=metastasis-free).

FIGS. 10A-10C. Kaplan-Meier plots of the metastasis free probability as a function of time since initial diagnosis. Patients with breast cancer in the age group >55 years are classified as either “poor prognosis” or “good prognosis” group based on a classifier with 70 genes derived from data of age group <55 years in our previous study (FIG. 10A), a classifier with 200 genes built with the same method but based on this data set (FIG. 10B), and a classifier with 100 genes built with a new method that looks for homogenous patterns in each group based on this data set (FIG. 10C).

FIGS. 11A, 11B. Classification error rate for type 1 and type 2 together as a function of the number of discriminating genes used in the classifier for ER+ sample group by the previously-published method (Van't Veer (2002); FIG. 11A); and by the iterative method (see Example 3; FIG. 11B).

FIG. 12. Gene expression pattern of 200 genes identified by the previously-published algorithm (Van't Veer (2002)) that can be used to predict the disease outcome for ER+ patient population (118 samples in the present study). The panel on the right indicates which samples are metastasis-positive (=1) and metastasis-free (═O).

FIG. 13. Gene expression pattern of 100 genes identified by the iterative algorithm that can be used to predict the disease outcome for ER+ patient population (118 samples in the present study. The panel on the right indicates which samples are metastasis-positive (=1) and metastasis-free (=0).

FIGS. 14A-14C. Kaplan-Meier plots of the metastasis free probability as a function of time since initial diagnosis. Patients with breast cancer in the age group >55 years and ER+ (118 patients total) are classified into a “poor prognosis” group and a “good prognosis” group based on a classifier with 70 genes derived from data of age group <55 years in our previous study (FIG. 14A); a classifier with 200 genes built with the same method but based on this data set (FIG. 14B); and a classifier with 100 genes build with an iterative method (Example 3) that looks for homogenous patterns in each group based on this data set (FIG. 14C).

### 5. DETAILED DESCRIPTION OF THE INVENTION

### 5.1 Markers Useful in the Prognosis of Breast Cancer

### 5.1.1 Definitions

As used herein, “age 55+ individuals” means individuals that are age 55 or older.

As used herein, “BRCA1 tumor” means a tumor having cells containing a mutation of the BRCA1 locus.

The “absolute amplitude” of a correlation coefficient means the absolute value of the correlation coefficient, e.g., both correlation coefficients −0.35 and 0.35 have an absolute amplitude of 0.35.

“Good prognosis” means a desired outcome. For example, in the context of breast cancer, a good prognosis may be an expectation of no reoccurrences or metastasis within two, three, four, five years or more of initial diagnosis of breast cancer.

“Poor prognosis” means an undesired outcome. For example, in the context of breast cancer, a poor prognosis may be an expectation of a reoccurrence or metastasis within two, three, four, or five years of initial diagnosis of breast cancer.

“Marker” means a gene or gene products, or an EST derived from that gene, the expression or level of which changes between certain conditions. Where the expression of the gene or gene products correlates with a certain condition, the gene or its products are a marker for that condition.

“Marker-derived polynucleotides” means the RNA transcribed from a marker gene, any cDNA or cRNA produced therefrom, and any nucleic acid derived therefrom, such as synthetic nucleic acid having a sequence derived from the marker gene.

As used herein, “prognosis informative” means statistically significantly correlated. For example, the expression of a particular gene is prognosis-informative if its expression is significantly correlated with either a good prognosis or a poor prognosis.

A “similarity value” is a number that represents the degree of similarity between two things being compared. For example, a similarity value may be a number that indicates the overall similarity between a patient's expression profile using specific phenotype-related markers and a control specific to that phenotype (for instance, the similarity to a “good prognosis” template, where the phenotype is a good prognosis). The similarity value may be expressed as a similarity metric, such as a correlation coefficient, or may simply be expressed as the expression level difference, or the aggregate of the expression level differences, between a patient sample and a template.

ER designates the estrogen receptor status of a breast cancer patient. ER⁺ designates a high ER level, while ER⁻ designates a low ER level. The ER status of a breast cancer patient can be evaluated by various means. In one embodiment, the ER level is determined by measuring an expression level of a gene encoding the estrogen receptor in a patient. In one embodiment, the gene encoding the estrogen receptor is the estrogen receptor a gene. In a specific embodiment, the expression level of the estrogen receptor a gene in the patient relative to the expression level of the gene in a pool of breast tumor samples is used as a measure of the ER status, and the ER level is classified as ER⁺ if the log 10(ratio) of the expression level is greater than −0.65, and the ER level is classified as ER⁻ if the log 10(ratio) of the expression level is equal to or less than −0.65. In another embodiment, the ER status is evaluated based on the expression profile of a set of marker genes as described in PCT Publication No. WO 02/103320.

### 5.1.2 Marker Sets

The invention provides sets of genetic markers whose expression is correlated with the prognosis of breast cancer. These markers are listed as SEQ ID NOS: 1-387 herein. These markers are particularly useful in the prognosis of breast cancer in individuals of age 55 or older.

In one embodiment, the invention provides a set of 387 breast cancer prognosis-informative markers, i.e., markers that are significantly correlated with either a good or a poor outcome in breast cancer patients. These markers are listed in Tables 1, 3, 5 and 7 or in Tables 2, 4, 6 and 8. Tables 1 and 2 list the same markers; Tables 1, 3, 5 and 7 correlate particular markers with SEQ ID NOS for the 387 markers, and Tables 2, 4, 6 and 8 provide gene names and descriptions for each of the 387 markers. The invention also provides subsets of at least 10, 15, 20, 25, 30, 40, 50, 75, 100, 125, 150, or 175 of the different markers present in Tables 1, 3, 5 and 7 or in Tables 2, 4, 6 and 8, which are useful for prognosis of breast cancer in individuals having breast cancer, including age 55+ individuals. The invention further provides subsets of no more than 10, 15, 20, 25, 30, 40, 50, 75, 100, 125, 150, or 175 of the different markers in Tables 1, 3, 5 and 7 or in Tables 2, 4, 6 and 8, which are useful for prognosis of breast cancer in individuals having breast cancer, including age 55+ individuals. The invention further provides subsets of at least 10%, 15%, 20%, 25%, 30%, 40%, 50%, 60%, 70%, 80%, or 90% of the different markers listed in Tables 1, 3, 5 and 7 or in Tables 2, 4, 6 and 8. Preferably, a subset comprises all 387 different markers listed in Tables 1, 3, 5 and 7 or in Tables 2, 4, 6 and 8.

In one embodiment, the invention provides a set of 200 breast cancer prognosis-informative markers, i.e., markers that are significantly correlated with either a good or a poor outcome in breast cancer patients. These markers were identified using an algorithm previously described (see International Application Publication No. WO 02/103320), and are listed in Tables 1 and 2. Tables 1 and 2 list the same markers; Table 1 correlates particular markers with SEQ ID NOS for the 200 markers, and Table 2 provides gene names and descriptions for each of the 200 markers. The invention also provides subsets of at least 10, 15, 20, 25, 30, 40, 50, 75, 100, 125, 150, or 175 of the markers present in Tables 1 or 2, which are useful for prognosis of breast cancer in individuals having breast cancer, including age 55+ individuals. The invention further provides subsets of no more than 10, 15, 20, 25, 30, 40, 50, 75, 100, 125, 150, or 175 of the markers listed in Tables 1 or 2, which are useful for prognosis of breast cancer in individuals having breast cancer, including age 55+ individuals. The invention further provides subsets of at least 10%, 15%, 20%, 25%, 30%, 40%, 50%, 60%, 70%, 80%, or 90% of the markers listed in Table 1 or 2. Preferably, a subset comprises 100 of the markers, and even more preferably comprises all 200 markers listed in Table 1 or 2.

In another embodiment, the invention provides a set of 100 breast cancer prognosis-informative markers. These markers were identified by an iterative sample-exclusion method described elsewhere herein (see Example 3). These markers are listed in both Tables 3 and 4; Table 3 correlates particular markers with SEQ ID NOS for the 100 markers, and Table 4 provides gene names and descriptions for each of the 100 markers. The invention also provides subsets of at least 10, 15, 20, 25, 30, 40, 50 or 75 of the markers present in Table 3 or 4, which are useful for prognosis of breast cancer in individuals having breast cancer, including age 55+ individuals. The invention further provides subsets of no more than 10, 15, 20, 25, 30, 40, 50 or 75 of the markers present in Table 3 or 4, which are useful for prognosis of breast cancer in individuals having breast cancer, including age 55+ individuals. The invention further provides subsets of at least 10%, 15%, 20%, 25%, 30%, 40%, 50%, 60%, 70%, 80%, or 90% of the markers listed in Table 3 or 4. Preferably, a subset comprises 50 of the markers, and even more preferably comprises all 100 markers listed in Table 3 or 4.

In another embodiment, the invention provides a set of 200 breast cancer prognosis-informative markers, i.e., markers that are significantly correlated with either a good or a poor prognosis. These markers were identified using an algorithm previously described (see International Application Publication No. WO 02/103320) applied to samples from individuals with ER+ tumors. These markers are listed in Table 5 and 6. Table 5 and 6 list the same markers; Table 5 correlates particular markers with SEQ ID NOS for the 200 markers, and Table 6 provides gene names and descriptions for each of the 200 markers. The invention also provides subsets of at least 10, 15, 20, 25, 30, 40, 50, 75, 100, 125, 150, or 175 of the markers present in Table 5 or 6, which are particularly useful for prognosis of breast cancer in individuals having breast cancer, including age 55+, ER+ individuals. The invention further provides subsets of no more than 10, 15, 20, 25, 30, 40, 50, 75, 100, 125, 150, or 175 of the markers present in Table 5 or 6, which are particularly useful for prognosis of breast cancer in individuals having breast cancer, including age 55+ individuals with ER+ tumors. The invention further provides subsets of at least 10%, 15%, 20%, 25%, 30%, 40%, 50%, 60%, 70%, 80%, or 90% of the markers listed in Table 5 or 6. Preferably, a subset comprises 100 of the markers, and even more preferably comprises all 200 markers listed in Table 5 or 6.

In another embodiment, the invention provides a set of 100 breast cancer prognosis-informative markers. These markers were identified by an iterative sample-exclusion method described elsewhere herein (see Example 3) using ER+tumor samples. These markers are listed in both Table 7 and 8; Table 7 correlates particular markers with SEQ ID NOS for the 100 markers, and Table 8 provides gene names and descriptions for each of the 100 markers. The invention also provides subsets of at least 10, 15, 20, 25, 30, 40, 50 or 75 of the markers present in Table 7 or 8, which are useful for prognosis of breast cancer in individuals having breast cancer, including age 55+ individuals having ER+ tumors. The invention further provides subsets of no more than 10, 15, 20, 25, 30, 40, 50 or 75 of the markers present in Table 7 or 8, which are useful for prognosis of breast cancer in individuals having breast cancer, including age 55+ individuals having ER+ tumors. The invention further provides subsets of at least 10%, 15%, 20%, 25%, 30%, 40%, 50%, 60%, 70%, 80%, or 90% of the markers listed in Table 7 or 8. Preferably, a subset comprises 50 of the markers, and even more preferably comprises all 100 markers listed in Table 7 or 8.

In another embodiment, the invention provides subsets of at least 10, 15, 20, 25, 30, 40, 50, 75, 100, 125, 150, 175, 200, 225, 275, 300 or 350 of the markers listed in any one or more of Tables 1, 3, 5, and 7, or in any one or more of Tables 2, 4, 6, and 8. The invention further provides subsets of at least 10%, 15%, 20%, 25%, 30%, 40%, 50%, 60%, 70%, 80%, or 90% of the markers listed in any one or more of Tables 1, 3, 5, and 7, or in any one or more of Tables 2, 4, 6, and 8; that is, 10%, 15%, 20%, 25%, 30%, 40%, 50%, 60%, 70%, 80%, or 90% of the sequences of SEQ ID NOS:1-387. That is, prognosis-informative markers may be selected from any one or more of Tables 1, 3, 5, and 7, or any one or more of Tables 2, 4, 6, and 8, and used in the methods of the invention. In specific embodiments, preferred prognosis-informative markers are those derived from genes that encode kinases or cell cycle control proteins.

TABLE 1
200 prognosis markers identified by clustering method
previously described (“Nature method”)(Van't Veer et al. Nature
415(6871): 530-536(2002)).
Table 1

| Accession/ | |
|---|---|
| Contig No. | SEQ ID NO.: |
| AB007913 | SEQ ID NO 1 |
| AB029000 | SEQ ID NO 3 |
| AB033006 | SEQ ID NO 4 |
| AB033025 | SEQ ID NO 5 |
| AB033090 | SEQ ID NO 7 |
| AB033117 | SEQ ID NO 8 |
| AB037734 | SEQ ID NO 9 |
| AB037805 | SEQ ID NO 10 |
| AB040912 | SEQ ID NO 11 |
| AF052162 | SEQ ID NO 16 |
| AF067972 | SEQ ID NO 18 |
| AF121255 | SEQ ID NO 21 |
| AF154121 | SEQ ID NO 23 |
| AK000770 | SEQ ID NO 24 |
| AL050015 | SEQ ID NO 31 |
| AL050021 | SEQ ID NO 32 |
| AL080065 | SEQ ID NO 33 |
| AL080199 | SEQ ID NO 35 |
| AL117544 | SEQ ID NO 39 |
| AL133017 | SEQ ID NO 42 |
| AL133092 | SEQ ID NO 44 |
| AL137379 | SEQ ID NO 45 |
| AL137566 | SEQ ID NO 46 |
| AL137698 | SEQ ID NO 47 |
| D14678 | SEQ ID NO 49 |
| D25304 | SEQ ID NO 50 |
| D25328 | SEQ ID NO 51 |
| D42046 | SEQ ID NO 53 |
| D55716 | SEQ ID NO 54 |
| L19778 | SEQ ID NO 57 |
| L35035 | SEQ ID NO 59 |
| NM_000114 | SEQ ID NO 64 |
| NM_000147 | SEQ ID NO 65 |
| NM_000150 | SEQ ID NO 66 |
| NM_000587 | SEQ ID NO 69 |
| NM_000785 | SEQ ID NO 71 |
| NM_000876 | SEQ ID NO 73 |
| NM_000926 | SEQ ID NO 75 |
| NM_000927 | SEQ ID NO 76 |
| NM_000988 | SEQ ID NO 77 |
| NM_001034 | SEQ ID NO 78 |
| NM_001128 | SEQ ID NO 80 |
| NM_001310 | SEQ ID NO 85 |
| NM_001605 | SEQ ID NO 87 |
| NM_001673 | SEQ ID NO 89 |
| NM_001723 | SEQ ID NO 90 |
| NM_001762 | SEQ ID NO 91 |
| NM_001765 | SEQ ID NO 92 |
| NM_002001 | SEQ ID NO 97 |
| NM_002036 | SEQ ID NO 98 |
| NM_002428 | SEQ ID NO 104 |
| NM_002624 | SEQ ID NO 106 |
| NM_002837 | SEQ ID NO 107 |
| NM_002875 | SEQ ID NO 108 |
| NM_003005 | SEQ ID NO 110 |
| NM_003152 | SEQ ID NO 113 |
| NM_003160 | SEQ ID NO 114 |
| NM_003183 | SEQ ID NO 115 |
| NM_003243 | SEQ ID NO 117 |
| NM_003364 | SEQ ID NO 119 |
| NM_003504 | SEQ ID NO 120 |
| NM_003551 | SEQ ID NO 121 |
| NM_003559 | SEQ ID NO 122 |
| NM_003600 | SEQ ID NO 124 |
| NM_003686 | SEQ ID NO 126 |
| NM_003722 | SEQ ID NO 127 |
| NM_003864 | SEQ ID NO 130 |
| NM_003956 | SEQ ID NO 131 |
| NM_003981 | SEQ ID NO 133 |
| NM_004162 | SEQ ID NO 135 |
| NM_004203 | SEQ ID NO 136 |
| NM_004216 | SEQ ID NO 137 |
| NM_004217 | SEQ ID NO 138 |
| NM_004456 | SEQ ID NO 141 |
| NM_004526 | SEQ ID NO 142 |
| NM_004527 | SEQ ID NO 143 |
| NM_004603 | SEQ ID NO 144 |
| NM_004615 | SEQ ID NO 145 |
| NM_004642 | SEQ ID NO 147 |
| NM_004701 | SEQ ID NO 148 |
| NM_004774 | SEQ ID NO 149 |
| NM_004827 | SEQ ID NO 151 |
| NM_004861 | SEQ ID NO 152 |
| NM_004867 | SEQ ID NO 153 |
| NM_004944 | SEQ ID NO 155 |
| NM_005176 | SEQ ID NO 157 |
| NM_005269 | SEQ ID NO 160 |
| NM_005447 | SEQ ID NO 161 |
| NM_005721 | SEQ ID NO 164 |
| NM_005792 | SEQ ID NO 166 |
| NM_005856 | SEQ ID NO 167 |
| NM_006141 | SEQ ID NO 171 |
| NM_006461 | SEQ ID NO 175 |
| NM_006636 | SEQ ID NO 177 |
| NM_006826 | SEQ ID NO 179 |
| NM_006829 | SEQ ID NO 180 |
| NM_007057 | SEQ ID NO 184 |
| NM_012087 | SEQ ID NO 187 |
| NM_012291 | SEQ ID NO 189 |
| NM_012394 | SEQ ID NO 191 |
| NM_012453 | SEQ ID NO 194 |
| NM_012464 | SEQ ID NO 195 |
| NM_013242 | SEQ ID NO 196 |
| NM_013277 | SEQ ID NO 199 |
| NM_013999 | SEQ ID NO 201 |
| NM_014176 | SEQ ID NO 204 |
| NM_014251 | SEQ ID NO 205 |
| NM_014370 | SEQ ID NO 208 |
| NM_014669 | SEQ ID NO 209 |
| NM_014791 | SEQ ID NO 213 |
| NM_014926 | SEQ ID NO 214 |
| NM_015710 | SEQ ID NO 216 |
| NM_015969 | SEQ ID NO 217 |
| NM_015982 | SEQ ID NO 218 |
| NM_015987 | SEQ ID NO 219 |
| NM_017697 | SEQ ID NO 230 |
| NM_017870 | SEQ ID NO 231 |
| NM_017888 | SEQ ID NO 232 |
| NM_017899 | SEQ ID NO 233 |
| NM_017975 | SEQ ID NO 234 |
| NM_018114 | SEQ ID NO 237 |
| NM_018655 | SEQ ID NO 246 |
| NM_018659 | SEQ ID NO 247 |
| NM_018944 | SEQ ID NO 248 |
| NM_019013 | SEQ ID NO 249 |
| NM_020397 | SEQ ID NO 250 |
| NM_020980 | SEQ ID NO 252 |
| NM_020990 | SEQ ID NO 253 |
| NM_021128 | SEQ ID NO 256 |
| NM_021225 | SEQ ID NO 258 |
| S62027 | SEQ ID NO 259 |
| U20180 | SEQ ID NO 260 |
| U96131 | SEQ ID NO 264 |
| V00522 | SEQ ID NO 265 |
| Z26649 | SEQ ID NO 267 |
| NM_003158 | SEQ ID NO 269 |
| AW190932_RC | SEQ ID NO 270 |
| BE675111_RC | SEQ ID NO 271 |
| NM_018605 | SEQ ID NO 272 |
| NM_018692 | SEQ ID NO 273 |
| Contig1352_RC | SEQ ID NO 274 |
| Contig1938_RC | SEQ ID NO 275 |
| Contig3677_RC | SEQ ID NO 277 |
| Contig6796_RC | SEQ ID NO 278 |
| Contig8373_RC | SEQ ID NO 280 |
| Contig9810_RC | SEQ ID NO 281 |
| Contig32810 | SEQ ID NO 283 |
| Contig34952 | SEQ ID NO 284 |
| Contig35752 | SEQ ID NO 285 |
| Contig37540 | SEQ ID NO 286 |
| Contig47129 | SEQ ID NO 287 |
| Contig49875 | SEQ ID NO 288 |
| Contig50368 | SEQ ID NO 289 |
| Contig56307 | SEQ ID NO 290 |
| Contig62306 | SEQ ID NO 291 |
| Contig10007_RC | SEQ ID NO 292 |
| Contig13866_RC | SEQ ID NO 293 |
| Contig15750_RC | SEQ ID NO 294 |
| Contig16098_RC | SEQ ID NO 295 |
| Contig18374_RC | SEQ ID NO 297 |
| Contig24565_RC | SEQ ID NO 303 |
| Contig26492_RC | SEQ ID NO 307 |
| Contig27623_RC | SEQ ID NO 308 |
| Contig28286_RC | SEQ ID NO 309 |
| Contig29543_RC | SEQ ID NO 311 |
| Contig31885_RC | SEQ ID NO 315 |
| Contig34019_RC | SEQ ID NO 318 |
| Contig34154_RC | SEQ ID NO 319 |
| Contig34667_RC | SEQ ID NO 321 |
| Contig34766_RC | SEQ ID NO 322 |
| Contig35030_RC | SEQ ID NO 323 |
| Contig36243_RC | SEQ ID NO 324 |
| Contig37966_RC | SEQ ID NO 326 |
| Contig38901_RC | SEQ ID NO 328 |
| Contig39031_RC | SEQ ID NO 329 |
| Contig39795_RC | SEQ ID NO 330 |
| Contig41413_RC | SEQ ID NO 333 |
| Contig42103_RC | SEQ ID NO 336 |
| Contig43747_RC | SEQ ID NO 339 |
| Contig43898_RC | SEQ ID NO 341 |
| Contig44291_RC | SEQ ID NO 343 |
| Contig45441_RC | SEQ ID NO 345 |
| Contig45565_RC | SEQ ID NO 346 |
| Contig47308_RC | SEQ ID NO 352 |
| Contig48215_RC | SEQ ID NO 354 |
| Contig49270_RC | SEQ ID NO 356 |
| Contig49279_RC | SEQ ID NO 357 |
| Contig50900_RC | SEQ ID NO 363 |
| Contig52193_RC | SEQ ID NO 366 |
| Contig52543_RC | SEQ ID NO 368 |
| Contig53180_RC | SEQ ID NO 369 |
| Contig54096_RC | SEQ ID NO 370 |
| Contig54666_RC | SEQ ID NO 371 |
| Contig54895_RC | SEQ ID NO 372 |
| Contig55725_RC | SEQ ID NO 376 |
| Contig55883_RC | SEQ ID NO 377 |
| Contig55997_RC | SEQ ID NO 378 |
| Contig56298_RC | SEQ ID NO 379 |
| Contig56852_RC | SEQ ID NO 381 |
| Contig63649_RC | SEQ ID NO 387 |

TABLE 2
Accession/contig number, gene name, correlation to prognosis, and description for
each of the markers listed in Table 1.
Table 2.

| Accession/ | | | | |
|---|---|---|---|---|
| Contig No. | Gene | Corr | Name | Description |
| Z26649 | PLCB3 | 0.46 | PLCB3 | phospholipase C, beta 3 |
| | | | | (phosphatidylinositol-specific) |
| AF052162 | FLJ12443 | 0.43 | FLJ12443 | hypothetical protein FLJ12443 |
| AK000770 | B3GNT7 | 0.42 | B3GNT7 | UDP-GlcNAc:betaGal beta-1,3-N- |
| | | | | acetylglucosaminyltransferase 7 |
| AL133017 | FLJ22865 | 0.41 | FLJ22865 | hypothetical protein FLJ22865 |
| NM_019013 | FLJ10156 | 0.4 | FLJ10156 | hypothetical protein FLJ10156 |
| NM_001034 | RRM2 | 0.4 | RRM2 | ribonucleotide reductase M2 polypeptide |
| Contig54895_RC | FLJ12691 | 0.4 | GALNT14 | UDP-N-acetyl-alpha-D- |
| | | | | galactosamine:polypeptide N- |
| | | | | acetylgalactosaminyltransferase 14 |
| | | | | (GalNAc-T14) |
| AL137379 | FLJ13912 | 0.39 | FLJ13912 | hypothetical protein FLJ13912 |
| Contig38901_RC | MGC45866 | 0.38 | MGC45866 | hypothetical protein MGC45866 |
| Contig41413_RC | RRM2 | 0.38 | RRM2 | ribonucleotide reductase M2 polypeptide |
| NM_006636 | MTHFD2 | 0.38 | MTHFD2 | methylene tetrahydrofolate |
| | | | | dehydrogenase (NAD+ dependent), |
| | | | | methenyltetrahydrofolate cyclohydrolase |
| D25328 | PFKP | 0.38 | PFKP | phosphofructokinase, platelet |
| NM_013242 | GTL3 | 0.38 | GTL3 | likely ortholog of mouse gene trap locus 3 |
| NM_003600 | STK6 | 0.37 | STK6 | serine/threonine kinase 6 |
| NM_002875 | RAD51 | 0.37 | RAD51 | RAD51 homolog (RecA homolog, E. coli) |
| | | | | (S. cerevisiae) |
| NM_014791 | MELK | 0.37 | MELK | maternal embryonic leucine zipper kinase |
| NM_004217 | STK12 | 0.37 | AURKB | aurora kinase B |
| NM_004603 | STX1A | 0.37 | STX1A | syntaxin 1A (brain) |
| Contig35752 | CUL1 | 0.37 | CUL1 | cullin 1 |
| NM_003364 | UP | 0.37 | UPP1 | uridine phosphorylase 1 |
| Contig16098_RC | | 0.37 | | Homo sapiens transcribed sequences |
| AW190932_RC | GPI | 0.37 | GPI | xl66g09.x1 NCI_CGAP_Pan1 Homo |
| | | | | sapiens cDNA clone IMAGE: 2679712 3′, |
| | | | | mRNA sequence. |
| NM_003158 | STK6 | 0.36 | STK6 | Homo sapiens mRNA for aurora/IPL1- |
| | | | | related kinase, complete cds. |
| NM_020990 | CKMT1 | 0.36 | CKMT1 | creatine kinase, mitochondrial 1 |
| | | | | (ubiquitous) |
| Contig53180_RC | ADCY3 | 0.36 | MGC11266 | hypothetical protein MGC11266 |
| NM_003559 | PIP5K2B | 0.36 | PIP5K2B | phosphatidylinositol-4-phosphate 5- |
| | | | | kinase, type II, beta |
| Contig63649_RC | | 0.36 | | Homo sapiens cDNA FLJ41489 fis, clone |
| | | | | BRTHA2004582 |
| NM_006461 | SPAG5 | 0.35 | SPAG5 | sperm associated antigen 5 |
| NM_004642 | CDK2AP1 | 0.35 | CDK2AP1 | CDK2-associated protein 1 |
| NM_005792 | MPHOSPH6 | 0.35 | MPHOSPH6 | M-phase phosphoprotein 6 |
| Contig18374_RC | LYPLA3 | 0.35 | LYPLA3 | lysophospholipase 3 (lysosomal |
| | | | | phospholipase A2) |
| NM_003183 | ADAM17 | 0.35 | ADAM17 | a disintegrin and metalloproteinase |
| | | | | domain 17 (tumor necrosis factor, alpha, |
| | | | | converting enzyme) |
| NM_015969 | MRPS17 | 0.35 | MRPS17 | mitochondrial ribosomal protein S17 |
| NM_017975 | FLJ10036 | 0.35 | FLJ10036 | hypothetical protein FLJ10036 |
| Contig39031_RC | FLJ37312 | 0.35 | PTP9Q22 | protein tyrosine phosphatase PTP9Q22 |
| AF154121 | SLC13A3 | 0.35 | SLC13A3 | solute carrier family 13 (sodium- |
| | | | | dependent dicarboxylate transporter), |
| | | | | member 3 |
| Contig28286_RC | | 0.35 | | Homo sapiens transcribed sequences |
| NM_004216 | DEDD | 0.35 | DEDD | death effector domain containing |
| Contig13866_RC | | 0.35 | | Homo sapiens transcribed sequences |
| Contig49270_RC | KIAA1553 | 0.34 | KIAA1553 | KIAA1553 |
| D42046 | DNA2L | 0.34 | DNA2L | DNA2 DNA replication helicase 2-like |
| | | | | (yeast) |
| AF067972 | DNMT3A | 0.34 | DNMT3A | DNA (cytosine-5-)-methyltransferase 3 |
| | | | | alpha |
| Contig47129 | MGC22014 | 0.34 | MGC22014 | hypothetical protein MGC22014 |
| Contig35030_RC | LOC91689 | 0.34 | RDH10 | retinol dehydrogenase 10 (all-trans) |
| AB033006 | NDRG4 | 0.34 | NDRG4 | NDRG family member 4 |
| NM_003864 | SAP30 | 0.34 | SAP30 | sin3-associated polypeptide, 30 kDa |
| Contig56298_RC | FLJ13154 | 0.34 | FLJ13154 | hypothetical protein FLJ13154 |
| NM_018114 | WDR6 | 0.34 | FLJ10496 | hypothetical protein FLJ10496 |
| BE675111_RC | FLJ20374 | 0.34 | Rpp25 | RNase P protein subunit p25 |
| NM_015982 | YBX2 | 0.34 | YBX2 | germ cell specific Y-box binding protein |
| Contig34019_RC | MGC15827 | 0.34 | MGC15827 | hypothetical protein MGC15827 |
| NM_004861 | CST | 0.34 | CST | cerebroside (3′- |
| | | | | phosphoadenylylsulfate:galactosylceramide |
| | | | | 3′) sulfotransferase |
| AB033117 | XPO5 | 0.34 | XPO5 | exportin 5 |
| Contig44291_RC | FLJ21415 | 0.34 | FLJ21415 | hypothetical protein FLJ21415 |
| Contig29543_RC | 0.34 | FLJ30594 | hypothetical | |
| | | | protein | |
| | | | FLJ30594 | |
| NM_014370 | STK23 | 0.34 | STK23 | serine/threonine kinase 23 |
| Contig6796_RC | 0.34 | | Homo | |
| | | | sapiens | |
| | | | transcribed | |
| | | | sequences | |
| NM_014176 | HSPC150 | 0.33 | HSPC150 | HSPC150 protein similar to ubiquitin- |
| | | | | conjugating enzyme |
| D14678 | KIFC1 | 0.33 | KIFC1 | kinesin family member C1 |
| NM_001762 | CCT6A | 0.33 | CCT6A | chaperonin containing TCP1, subunit 6A |
| | | | | (zeta 1) |
| Contig3677_RC | CBFB | 0.33 | CBFB | core-binding factor, beta subunit |
| NM_017697 | FLJ20171 | 0.33 | FLJ20171 | hypothetical protein FLJ20171 |
| AL050021 | | 0.33 | SLC7A1 | solute carrier family 7 (cationic amino |
| | | | | acid transporter, y+ system), member 1 |
| NM_014251 | SLC25A13 | 0.33 | SLC25A13 | solute carrier family 25, member 13 |
| | | | | (citrin) |
| NM_014669 | KIAA0095 | 0.33 | KIAA0095 | KIAA0095 gene product |
| Contig37966_RC | MGC15482 | 0.33 | MGC15482 | F-box protein FBL2 |
| Contig54096_RC | CKMT1 | 0.33 | CKMT1 | creatine kinase, mitochondrial 1 |
| | | | | (ubiquitous) |
| NM_001128 | AP1G1 | 0.33 | AP1G1 | adaptor-related protein complex 1, |
| | | | | gamma 1 subunit |
| NM_001605 | AARS | 0.33 | AARS | alanyl-tRNA synthetase |
| NM_000785 | CYP27B1 | 0.33 | CYP27B1 | cytochrome P450, family 27, subfamily B, |
| | | | | polypeptide 1 |
| NM_012453 | TBL2 | 0.33 | TBL2 | transducin (beta)-like 2 |
| U20180 | IREB2 | 0.33 | IREB2 | iron-responsive element binding protein 2 |
| NM_003160 | STK13 | 0.33 | AURKC | aurora kinase C |
| NM_007057 | ZWINT | 0.33 | ZWINT | ZW10 interactor |
| NM_004203 | RBL2 | 0.33 | PKMYT1 | membrane-associated tyrosine- and |
| | | | | threonine-specific cdc2-inhibitory kinase |
| NM_012394 | PFDN2 | 0.33 | PFDN2 | prefoldin 2 |
| NM_012087 | GTF3C5 | 0.33 | GTF3C5 | general transcription factor IIIC, |
| | | | | polypeptide 5, 63 kDa |
| Contig48215_RC | FLJ35801 | 0.33 | FLJ35801 | hypothetical protein FLJ35801 |
| NM_000150 | FUT6 | 0.33 | FUT6 | fucosyltransferase 6 (alpha (1,3) |
| | | | | fucosyltransferase) |
| Contig36243_RC | | 0.33 | DKFZP434A1022 | hypothetical protein DKFZp434A1022 |
| NM_004701 | CCNB2 | 0.32 | CCNB2 | cyclin B2 |
| NM_003981 | PRC1 | 0.32 | PRC1 | protein regulator of cytokinesis 1 |
| U96131 | TRIP13 | 0.32 | TRIP13 | thyroid hormone receptor interactor 13 |
| NM_003686 | EXO1 | 0.32 | EXO1 | exonuclease 1 |
| Contig34952 | SHCBP1 | 0.32 | SHCBP1 | likely ortholog of mouse Shc SH2-domain |
| | | | | binding protein 1 |
| NM_013277 | RACGAP1 | 0.32 | RACGAP1 | Rac GTPase activating protein 1 |
| NM_012291 | ESPL1 | 0.32 | ESPL1 | extra spindle poles like 1 (S. cerevisiae) |
| NM_005721 | ACTR3 | 0.32 | ACTR3 | ARP3 actin-related protein 3 homolog |
| | | | | (yeast) |
| NM_006826 | YWHAQ | 0.32 | YWHAQ | tyrosine 3-monooxygenase/tryptophan 5- |
| | | | | monooxygenase activation protein, theta |
| | | | | polypeptide |
| NM_004456 | EZH2 | 0.32 | EZH2 | enhancer of zeste homolog 2 |
| | | | | (Drosophila) |
| NM_004162 | RAB5A | 0.32 | RAB5A | RAB5A, member RAS oncogene family |
| Contig62306 | C21orf45 | 0.32 | C21orf45 | chromosome 21 open reading frame 45 |
| NM_001673 | ASNS | 0.32 | ASNS | asparagine synthetase |
| NM_020980 | AQP9 | 0.32 | AQP9 | aquaporin 9 |
| Contig50900_RC | LOC124491 | 0.32 | LOC124491 | LOC124491 |
| NM_018655 | LENEP | 0.32 | LENEP | lens epithelial protein |
| Contig1352_RC | FLJ36874 | 0.32 | FLJ36874 | hypothetical protein FLJ36874 |
| NM_020397 | CKLiK | 0.32 | CAMK1D | calcium/calmodulin-dependent protein |
| | | | | kinase ID |
| Contig34766_RC | LOC151648 | 0.32 | LOC151648 | hypothetical protein BC001339 |
| S62027 | GNGT1 | 0.32 | GNGT1 | guanine nucleotide binding protein (G |
| | | | | protein), gamma transducing activity |
| | | | | polypeptide 1 |
| NM_000876 | IGF2R | 0.32 | IGF2R | insulin-like growth factor 2 receptor |
| NM_002428 | MMP15 | 0.32 | MMP15 | matrix metalloproteinase 15 (membrane- |
| | | | | inserted) |
| Contig43898_RC | | 0.32 | FLJ39575 | hypothetical protein FLJ39575 |
| NM_003504 | CDC45L | 0.31 | CDC45L | CDC45 cell division cycle 45-like (S. cerevisiae) |
| Contig55997_RC | | 0.31 | | Homo sapiens cDNA clone |
| | | | | IMAGE: 4448513, partial cds |
| D55716 | MCM7 | 0.31 | MCM7 | MCM7 minichromosome maintenance |
| | | | | deficient 7 (S. cerevisiae) |
| NM_004526 | MCM2 | 0.31 | MCM2 | MCM2 minichromosome maintenance |
| | | | | deficient 2, mitotin (S. cerevisiae) |
| Contig55725_RC | CDCA7 | 0.31 | CDCA7 | cell division cycle associated 7 |
| L35035 | RPIA | 0.31 | RPIA | ribose 5-phosphate isomerase A (ribose |
| | | | | 5-phosphate epimerase) |
| NM_004774 | PPARBP | 0.31 | PPARBP | PPAR binding protein |
| NM_017870 | FLJ20539 | 0.31 | GBP | likely ortholog of rat GRP78-binding |
| | | | | protein |
| L19778 | HIST1H2AG | 0.31 | HIST1H2AG | histone 1, H2ag |
| AB033025 | KIAA1199 | 0.31 | KIAA1199 | KIAA1199 protein |
| AB029000 | KIAA1077 | 0.31 | SULF1 | sulfatase 1 |
| NM_006141 | DNCLI2 | 0.31 | DNCLI2 | dynein, cytoplasmic, light intermediate |
| | | | | polypeptide 2 |
| AF121255 | EIF2C2 | 0.31 | EIF2C2 | eukaryotic translation initiation factor 2C, 2 |
| NM_018944 | C21orf45 | 0.31 | C21orf45 | chromosome 21 open reading frame 45 |
| AB007913 | CHD5 | 0.31 | CHD5 | chromodomain helicase DNA binding |
| | | | | protein 5 |
| Contig24565_RC | MGC3077 | 0.31 | C7orf24 | chromosome 7 open reading frame 24 |
| Contig49875 | | −0.31 | | Homo sapiens full length insert cDNA |
| | | | | YN61C04 |
| NM_000927 | ABCB1 | −0.31 | ABCB1 | ATP-binding cassette, sub-family B |
| | | | | (MDR/TAP), member 1 |
| AB033090 | PAK7 | −0.31 | PAK7 | p21(CDKN1A)-activated kinase 7 |
| NM_021225 | PROL1 | −0.31 | PROL1 | proline rich 1 |
| Contig9810_RC | KCNE1 | −0.31 | KCNE1 | potassium voltage-gated channel, Isk- |
| | | | | related family, member 1 |
| Contig39795_RC | RASA1 | −0.31 | RASA1 | cyclin H |
| NM_001310 | CREBL2 | −0.31 | CREBL2 | cAMP responsive element binding |
| | | | | protein-like 2 |
| AL050015 | DKFZP564O243 | −0.31 | DKFZP564O243 | DKFZP564O243 protein |
| NM_002624 | PFDN5 | −0.31 | PFDN5 | prefoldin 5 |
| NM_021128 | POLR2L | −0.31 | POLR2L | polymerase (RNA) II (DNA directed) |
| | | | | polypeptide L, 7.6 kDa |
| NM_006829 | APM2 | −0.31 | APM2 | adipose specific 2 |
| NM_003005 | SELP | −0.31 | SELP | selectin P (granule membrane protein |
| | | | | 140 kDa, antigen CD62) |
| NM_012464 | TLL1 | −0.31 | TLL1 | tolloid-like 1 |
| NM_014926 | KIAA0848 | −0.31 | KIAA0848 | KIAA0848 protein |
| NM_000988 | RPL27 | −0.31 | RPL27 | ribosomal protein L27 |
| NM_001723 | BPAG1 | −0.32 | BPAG1 | bullous pemphigoid antigen 1, |
| | | | | 230/240 kDa |
| NM_018605 | PRO1777 | −0.32 | C13orf10 | chromosome 13 open reading frame 10 |
| AB037805 | KIAA1384 | −0.32 | KIAA1384 | KIAA1384 protein |
| Contig56852_RC | LRRN3 | −0.32 | LRRN3 | leucine rich repeat neuronal 3 |
| Contig31885_RC | | −0.32 | LOC147463 | hypothetical protein LOC147463 |
| NM_000147 | FUCA1 | −0.32 | FUCA1 | fucosidase, alpha-L-1, tissue |
| Contig34667_RC | LAK-4P | −0.32 | EVER1 | KIAA1582 protein |
| D25304 | ARHGEF6 | −0.32 | ARHGEF6 | Rac/Cdc42 guanine nucleotide exchange |
| | | | | factor (GEF) 6 |
| NM_005856 | RAMP3 | −0.32 | RAMP3 | receptor (calcitonin) activity modifying |
| | | | | protein 3 |
| NM_003551 | NME5 | −0.32 | NME5 | non-metastatic cells 5, protein expressed |
| | | | | in (nucleoside-diphosphate kinase) |
| V00522 | HLA-DRB3 | −0.32 | HLA-DRB3 | major histocompatibility complex, class II, |
| | | | | DR beta 3 |
| Contig54666_RC | C2orf9 | −0.32 | C2orf9 | chromosome 2 open reading frame 9 |
| AL133092 | DKFZp434I0428 | −0.32 | DISP1 | dispatched homolog 1 (Drosophila) |
| Contig32810 | | −0.32 | | Homo sapiens LOC374363 |
| | | | | (LOC374363), mRNA |
| AL137698 | DKFZp434C1915 | −0.32 | PGM5 | phosphoglucomutase 5 |
| Contig42103_RC | | −0.33 | C20orf17 | chromosome 20 open reading frame 17 |
| NM_001765 | CD1C | −0.33 | CD1C | CD1C antigen, c polypeptide |
| NM_018692 | C20orf17 | −0.33 | C20orf17 | chromosome 20 open reading frame 17 |
| NM_003722 | TP73L | −0.33 | TP73L | tumor protein p73-like |
| Contig15750_RC | | −0.33 | | Homo sapiens cDNA FLJ26876 fis, clone |
| | | | | PRS09003 |
| Contig55883_RC | | −0.33 | | Homo sapiens transcribed sequences |
| AL137566 | | −0.33 | | Homo sapiens mRNA; cDNA |
| | | | | DKFZp686A0815 (from clone |
| | | | | DKFZp686A0815) |
| NM_003243 | TGFBR3 | −0.33 | TGFBR3 | transforming growth factor, beta receptor |
| | | | | III (betaglycan, 300 kDa) |
| NM_005176 | ATP5G2 | −0.33 | ATP5G2 | ATP synthase, H+ transporting, |
| | | | | mitochondrial F0 complex, subunit c |
| | | | | (subunit 9), isoform 2 |
| Contig52543_RC | MGC29761 | −0.33 | MGC29761 | hypothetical protein MGC29761 |
| NM_005269 | GLI | −0.33 | GLI | glioma-associated oncogene homolog |
| | | | | (zinc finger protein) |
| NM_018659 | C17 | −0.33 | C17 | cytokine-like protein C17 |
| NM_017888 | FLJ20581 | −0.33 | FLJ20581 | hypothetical protein FLJ20581 |
| NM_004827 | ABCG2 | −0.33 | ABCG2 | ATP-binding cassette, sub-family G |
| | | | | (WHITE), member 2 |
| AL080065 | DKFZP564J102 | −0.33 | DKFZP564J102 | DKFZP564J102 protein |
| Contig27623_RC | | −0.33 | | Homo sapiens transcribed sequences |
| NM_002036 | FY | −0.34 | FY | Duffy blood group |
| Contig47308_RC | | −0.34 | | Homo sapiens hypothetical gene |
| | | | | supported by NM_018692 (LOC374296), |
| | | | | mRNA |
| Contig26492_RC | C12orf6 | −0.34 | C12orf6 | chromosome 12 open reading frame 6 |
| AL080199 | ELOVL2 | −0.34 | ELOVL2 | elongation of very long chain fatty acids |
| | | | | (FEN1/Elo2, SUR4/Elo3, yeast)-like 2 |
| NM_000926 | PGR | −0.34 | PGR | progesterone receptor |
| Contig34154_RC | | −0.34 | | Homo sapiens transcribed sequence with |
| | | | | strong similarity to protein |
| | | | | ref: NP_113668.1 (H. sapiens) |
| | | | | hypothetical protein AD034 [Homo |
| | | | | sapiens] |
| NM_003152 | STAT5A | −0.34 | STAT5A | signal transducer and activator of |
| | | | | transcription 5A |
| Contig43747_RC | MGC7036 | −0.34 | MGC7036 | hypothetical protein MGC7036 |
| Contig10007_RC | | −0.34 | | Homo sapiens similar to MHC HLA-SX- |
| | | | | alpha (LOC377373), mRNA |
| NM_013999 | MEOX1 | −0.35 | MEOX1 | mesenchyme homeo box 1 |
| NM_000587 | C7 | −0.35 | C7 | complement component 7 |
| Contig52193_RC | | −0.35 | ABCB1 | ATP-binding cassette, sub-family B |
| | | | | (MDR/TAP), member 1 |
| AB040912 | SEMA6D | −0.35 | SEMA6D | sema domain, transmembrane domain |
| | | | | (TM), and cytoplasmic domain, |
| | | | | (semaphorin) 6D |
| Contig56307 | C1orf21 | −0.35 | C1orf21 | chromosome 1 open reading frame 21 |
| AL117544 | DKFZP434I092 | −0.35 | DKFZP434I092 | DKFZP434I092 protein |
| NM_005447 | PAMCI | −0.35 | PAMCI | peptidylglycine alpha-amidating |
| | | | | monooxygenase COOH-terminal |
| | | | | interactor |
| Contig1938_RC | CAB56184 | −0.35 | CAB56184 | GlcNAc-phosphotransferase gamma- |
| | | | | subunit |
| NM_015710 | GLTSCR2 | −0.35 | GLTSCR2 | glioma tumor suppressor candidate |
| | | | | region gene 2 |
| NM_002837 | PTPRB | −0.35 | PTPRB | protein tyrosine phosphatase, receptor |
| | | | | type, B |
| Contig45565_RC | FLJ25162 | −0.35 | FLJ25162 | Homo sapiens cDNA FLJ25135 fis, clone |
| | | | | CBR06974. |
| NM_003956 | CH25H | −0.36 | CH25H | cholesterol 25-hydroxylase |
| Contig45441_RC | | −0.36 | LOC284542 | hypothetical protein LOC284542 |
| Contig37540 | | −0.36 | | Homo sapiens transcribed sequence with |
| | | | | weak similarity to protein |
| | | | | ref: NP_009056.1 (H. sapiens) |
| | | | | ubiquitously transcribed tetratricopeptide |
| | | | | repeat gene, Y chromosome; |
| | | | | Ubiquitously transcribed TPR gene on Y |
| | | | | chromosome [Homo sapiens] |
| Contig8373_RC | | −0.36 | | Homo sapiens transcribed sequence with |
| | | | | weak similarity to protein |
| | | | | ref: NP_060312.1 (H. sapiens) |
| | | | | hypothetical protein FLJ20489 [Homo |
| | | | | sapiens] |
| Contig50368 | | −0.36 | | cDNA encoding novel polypeptide from |
| | | | | human umbilical vein endothelial cell. |
| NM_000114 | EDN3 | −0.37 | EDN3 | endothelin 3 |
| NM_004944 | DNASE1L3 | −0.38 | DNASE1L3 | deoxyribonuclease I-like 3 |
| NM_017899 | TSC | −0.38 | TSC | hypothetical protein FLJ20607 |
| Contig49279_RC | | −0.38 | FLJ25461 | hypothetical protein FLJ25461 |
| NM_002001 | FCER1A | −0.38 | FCER1A | Fc fragment of IgE, high affinity I, |
| | | | | receptor for; alpha polypeptide |
| AB037734 | PCDH19 | −0.38 | PCDH19 | protocadherin 19 |
| NM_015987 | HEBP1 | −0.38 | HEBP1 | heme binding protein 1 |
| NM_004615 | TM4SF2 | −0.39 | TM4SF2 | transmembrane 4 superfamily member 2 |
| NM_004527 | MEOX1 | −0.39 | MEOX1 | mesenchyme homeo box 1 |
| NM_004867 | ITM2A | −0.39 | ITM2A | integral membrane protein 2A |

TABLE 3
100 prognosis markers identified by an iterative method.
Table 3.

| Accession/ | |
|---|---|
| Contig No. | SEQ ID NO.: |
| AB024704 | SEQ ID NO 2 |
| AF016495 | SEQ ID NO 13 |
| AF025441 | SEQ ID NO 14 |
| AF052162 | SEQ ID NO 16 |
| AK001166 | SEQ ID NO 25 |
| AL133017 | SEQ ID NO 42 |
| AL137698 | SEQ ID NO 47 |
| D14678 | SEQ ID NO 49 |
| D25328 | SEQ ID NO 51 |
| D38553 | SEQ ID NO 52 |
| D42046 | SEQ ID NO 53 |
| D55716 | SEQ ID NO 54 |
| D86978 | SEQ ID NO 55 |
| M96577 | SEQ ID NO 60 |
| NM_000291 | SEQ ID NO 67 |
| NM_001034 | SEQ ID NO 78 |
| NM_001071 | SEQ ID NO 79 |
| NM_001211 | SEQ ID NO 81 |
| NM_001237 | SEQ ID NO 83 |
| NM_001274 | SEQ ID NO 84 |
| NM_001762 | SEQ ID NO 91 |
| NM_001809 | SEQ ID NO 94 |
| NM_002001 | SEQ ID NO 97 |
| NM_002358 | SEQ ID NO 102 |
| NM_002466 | SEQ ID NO 105 |
| NM_002875 | SEQ ID NO 108 |
| NM_003090 | SEQ ID NO 112 |
| NM_003152 | SEQ ID NO 113 |
| NM_003318 | SEQ ID NO 118 |
| NM_003504 | SEQ ID NO 120 |
| NM_003551 | SEQ ID NO 121 |
| NM_003579 | SEQ ID NO 123 |
| NM_003600 | SEQ ID NO 124 |
| NM_003686 | SEQ ID NO 126 |
| NM_003981 | SEQ ID NO 133 |
| NM_004153 | SEQ ID NO 134 |
| NM_004217 | SEQ ID NO 138 |
| NM_004336 | SEQ ID NO 139 |
| NM_004456 | SEQ ID NO 141 |
| NM_004526 | SEQ ID NO 142 |
| NM_004527 | SEQ ID NO 143 |
| NM_004615 | SEQ ID NO 145 |
| NM_004631 | SEQ ID NO 146 |
| NM_004701 | SEQ ID NO 148 |
| NM_004887 | SEQ ID NO 154 |
| NM_005192 | SEQ ID NO 158 |
| NM_005721 | SEQ ID NO 164 |
| NM_005733 | SEQ ID NO 165 |
| NM_005915 | SEQ ID NO 168 |
| NM_006027 | SEQ ID NO 169 |
| NM_006461 | SEQ ID NO 175 |
| NM_006636 | SEQ ID NO 177 |
| NM_006845 | SEQ ID NO 181 |
| NM_007019 | SEQ ID NO 183 |
| NM_007057 | SEQ ID NO 184 |
| NM_012310 | SEQ ID NO 190 |
| NM_012412 | SEQ ID NO 192 |
| NM_013242 | SEQ ID NO 196 |
| NM_013277 | SEQ ID NO 199 |
| NM_014176 | SEQ ID NO 204 |
| NM_014251 | SEQ ID NO 205 |
| NM_014317 | SEQ ID NO 207 |
| NM_014736 | SEQ ID NO 210 |
| NM_014773 | SEQ ID NO 212 |
| NM_014791 | SEQ ID NO 213 |
| NM_015987 | SEQ ID NO 219 |
| NM_016101 | SEQ ID NO 221 |
| NM_016359 | SEQ ID NO 223 |
| NM_017613 | SEQ ID NO 227 |
| NM_018101 | SEQ ID NO 236 |
| NM_018131 | SEQ ID NO 238 |
| NM_018410 | SEQ ID NO 243 |
| NM_018518 | SEQ ID NO 245 |
| NM_019013 | SEQ ID NO 249 |
| NM_020675 | SEQ ID NO 251 |
| NM_020980 | SEQ ID NO 252 |
| U74612 | SEQ ID NO 262 |
| U81002 | SEQ ID NO 263 |
| U96131 | SEQ ID NO 264 |
| X74794 | SEQ ID NO 266 |
| NM_003158 | SEQ ID NO 269 |
| Contig173 | SEQ ID NO 282 |
| Contig34952 | SEQ ID NO 284 |
| Contig37540 | SEQ ID NO 286 |
| Contig28947_RC | SEQ ID NO 310 |
| Contig33814_RC | SEQ ID NO 317 |
| Contig34766_RC | SEQ ID NO 322 |
| Contig38901_RC | SEQ ID NO 328 |
| Contig40120_RC | SEQ ID NO 331 |
| Contig41413_RC | SEQ ID NO 333 |
| Contig45032_RC | SEQ ID NO 344 |
| Contig45816_RC | SEQ ID NO 347 |
| Contig47793_RC | SEQ ID NO 353 |
| Contig49270_RC | SEQ ID NO 356 |
| Contig50841_RC | SEQ ID NO 362 |
| Contig52543_RC | SEQ ID NO 368 |
| Contig54895_RC | SEQ ID NO 372 |
| Contig55725_RC | SEQ ID NO 376 |
| Contig55997_RC | SEQ ID NO 378 |
| Contig57584_RC | SEQ ID NO 382 |
| NM_006845 | SEQ ID NO 181 |
| NM_007019 | SEQ ID NO 183 |
| NM_007057 | SEQ ID NO 184 |
| NM_012310 | SEQ ID NO 190 |

TABLE 4
Accession/contig number, gene name, correlation to prognosis, and description for
each of the markers listed in Table 3.
Table 4.

| Accession/ | | | | |
|---|---|---|---|---|
| Contig No. | Gene | Corr | Name | Description |
| Contig41413_RC | RRM2 | 0.66 | RRM2 | ribonucleotide reductase M2 polypeptide |
| NM_014791 | MELK | 0.65 | MELK | maternal embryonic leucine zipper kinase |
| NM_004701 | CCNB2 | 0.65 | CCNB2 | cyclin B2 |
| NM_001034 | RRM2 | 0.65 | RRM2 | ribonucleotide reductase M2 polypeptide |
| Contig38901_RC | MGC45866 | 0.65 | MGC45866 | hypothetical protein MGC45866 |
| NM_003504 | CDC45L | 0.65 | CDC45L | CDC45 cell division cycle 45-like (S. cerevisiae) |
| NM_002875 | RAD51 | 0.63 | RAD51 | RAD51 homolog (RecA homolog, E. coli) |
| | | | | (S. cerevisiae) |
| NM_003158 | STK6 | 0.61 | STK6 | Homo sapiens mRNA for aurora/IPL1- |
| | | | | related kinase, complete cds. |
| NM_013277 | RACGAP1 | 0.61 | RACGAP1 | Rac GTPase activating protein 1 |
| NM_006636 | MTHFD2 | 0.61 | MTHFD2 | methylene tetrahydrofolate |
| | | | | dehydrogenase (NAD+ dependent), |
| | | | | methenyltetrahydrofolate cyclohydrolase |
| NM_003686 | EXO1 | 0.6 | EXO1 | exonuclease 1 |
| NM_012310 | KIF4A | 0.6 | KIF4A | kinesin family member 4A |
| NM_004217 | STK12 | 0.6 | AURKB | aurora kinase B |
| NM_002466 | MYBL2 | 0.6 | MYBL2 | v-myb myeloblastosis viral oncogene |
| | | | | homolog (avian)-like 2 |
| NM_003600 | STK6 | 0.59 | STK6 | serine/threonine kinase 6 |
| D55716 | MCM7 | 0.59 | MCM7 | MCM7 minichromosome maintenance |
| | | | | deficient 7 (S. cerevisiae) |
| NM_018410 | DKFZp762E1312 | 0.58 | DKFZp762E1312 | hypothetical protein DKFZp762E1312 |
| U96131 | TRIP13 | 0.58 | TRIP13 | thyroid hormone receptor interactor 13 |
| Contig55997_RC | | 0.58 | | Homo sapiens cDNA clone |
| | | | | IMAGE: 4448513, partial cds |
| NM_002358 | MAD2L1 | 0.58 | MAD2L1 | MAD2 mitotic arrest deficient-like 1 |
| | | | | (yeast) |
| NM_001237 | CCNA2 | 0.58 | CCNA2 | cyclin A2 |
| NM_017613 | DONSON | 0.58 | DONSON | downstream neighbor of SON |
| AB024704 | C20orf1 | 0.58 | C20orf1 | TPX2, microtubule-associated protein |
| | | | | homolog (Xenopus laevis) |
| Contig40120_RC | DCTN1 | 0.58 | SLC4A5 | solute carrier family 4, sodium |
| | | | | bicarbonate cotransporter, member 5 |
| NM_006027 | EXO1 | 0.57 | EXO1 | exonuclease 1 |
| NM_005733 | KIF20A | 0.57 | KIF20A | kinesin family member 20A |
| NM_003981 | PRC1 | 0.57 | PRC1 | protein regulator of cytokinesis 1 |
| NM_004456 | EZH2 | 0.57 | EZH2 | enhancer of zeste homolog 2 |
| | | | | (Drosophila) |
| Contig50841_RC | LOC113115 | 0.57 | DUFD1 | Homo sapiens DUF729 domain |
| | | | | containing 1, mRNA (cDNA clone |
| | | | | MGC: 19798 IMAGE: 3926284), complete |
| | | | | cds. |
| NM_019013 | FLJ10156 | 0.57 | FLJ10156 | hypothetical protein FLJ10156 |
| NM_004526 | MCM2 | 0.57 | MCM2 | MCM2 minichromosome maintenance |
| | | | | deficient 2, mitotin (S. cerevisiae) |
| NM_013242 | GTL3 | 0.57 | GTL3 | likely ortholog of mouse gene trap locus 3 |
| D25328 | PFKP | 0.57 | PFKP | phosphofructokinase, platelet |
| NM_004336 | BUB1 | 0.56 | BUB1 | BUB1 budding uninhibited by |
| | | | | benzimidazoles 1 homolog (yeast) |
| NM_001809 | CENPA | 0.56 | CENPA | centromere protein A, 17 kDa |
| D38553 | BRRN1 | 0.56 | BRRN1 | barren homolog (Drosophila) |
| NM_018518 | MCM10 | 0.56 | MCM10 | MCM10 minichromosome maintenance |
| | | | | deficient 10 (S. cerevisiae) |
| NM_016359 | ANKT | 0.56 | NUSAP1 | nucleolar and spindle associated protein 1 |
| Contig34952 | SHCBP1 | 0.56 | SHCBP1 | likely ortholog of mouse Shc SH2-domain |
| | | | | binding protein 1 |
| U74612 | FOXM1 | 0.56 | FOXM1 | forkhead box M1 |
| NM_003579 | RAD54L | 0.56 | RAD54L | RAD54-like (S. cerevisiae) |
| NM_018101 | FLJ10468 | 0.56 | CDCA8 | cell division cycle associated 8 |
| NM_006461 | SPAG5 | 0.56 | SPAG5 | sperm associated antigen 5 |
| X74794 | MCM4 | 0.56 | MCM4 | MCM4 minichromosome maintenance |
| | | | | deficient 4 (S. cerevisiae) |
| NM_004631 | LRP8 | 0.56 | LRP8 | low density lipoprotein receptor-related |
| | | | | protein 8, apolipoprotein e receptor |
| NM_014736 | KIAA0101 | 0.56 | KIAA0101 | KIAA0101 gene product |
| Contig34766_RC | LOC151648 | 0.56 | LOC151648 | hypothetical protein BC001339 |
| AF052162 | FLJ12443 | 0.56 | FLJ12443 | hypothetical protein FLJ12443 |
| NM_020675 | AD024 | 0.55 | AD024 | AD024 protein |
| Contig45032_RC | FLJ14813 | 0.55 | FLJ14813 | hypothetical protein FLJ14813 |
| NM_005192 | CDKN3 | 0.55 | CDKN3 | cyclin-dependent kinase inhibitor 3 |
| | | | | (CDK2-associated dual specificity |
| | | | | phosphatase) |
| Contig47793_RC | FLJ23311 | 0.55 | FLJ23311 | FLJ23311 protein |
| Contig57584_RC | GRCC8 | 0.55 | CDCA3 | cell division cycle associated 3 |
| Contig173 | C20orf178 | 0.55 | C20orf178 | dehydrogenase E1 and transketolase |
| | | | | domain containing 1 |
| NM_016101 | HSPC031 | 0.55 | CGI-37 | comparative gene identification transcript |
| | | | | 37 |
| NM_020980 | AQP9 | 0.55 | AQP9 | aquaporin 9 |
| NM_007057 | ZWINT | 0.55 | ZWINT | ZW10 interactor |
| NM_005915 | MCM6 | 0.54 | MCM6 | MCM6 minichromosame maintenance |
| | | | | deficient 6 (MIS5 homolog, S. pombe) (S. cerevisiae) |
| D42046 | DNA2L | 0.54 | DNA2L | DNA2 DNA replication helicase 2-like |
| | | | | (yeast) |
| U81002 | FLJ14502 | 0.54 | FLJ14502 | TRAF4 associated factor 1 |
| NM_014176 | HSPC150 | 0.54 | HSPC150 | HSPC150 protein similar to ubiquitin- |
| | | | | conjugating enzyme |
| NM_004153 | ORC1L | 0.54 | ORC1L | origin recognition complex, subunit 1-like |
| | | | | (yeast) |
| NM_005721 | ACTR3 | 0.54 | ACTR3 | ARP3 actin-related protein 3 homolog |
| | | | | (yeast) |
| Contig55725_RC | CDCA7 | 0.54 | CDCA7 | cell division cycle associated 7 |
| NM_001274 | CHEK1 | 0.54 | CHEK1 | CHK1 checkpoint homolog (S. pombe) |
| NM_014251 | SLC25A13 | 0.54 | SLC25A13 | solute carrier family 25, member 13 |
| | | | | (citrin) |
| Contig33814_RC | | 0.54 | ASPM | asp (abnormal spindle)-like, |
| | | | | microcephaly associated (Drosophila) |
| NM_003318 | TTK | 0.53 | TTK | TTK protein kinase |
| NM_007019 | UBE2C | 0.53 | UBE2C | ubiquitin-conjugating enzyme E2C |
| D14678 | KIFC1 | 0.53 | KIFC1 | kinesin family member C1 |
| Contig28947_RC | CDC25A | 0.53 | CDC25A | cell division cycle 25A |
| M96577 | E2F1 | 0.53 | E2F1 | E2F transcription factor 1 |
| NM_003090 | SNRPA1 | 0.53 | SNRPA1 | small nuclear ribonucleoprotein |
| | | | | polypeptide A′ |
| NM_014317 | TPT | 0.53 | TPT | trans-prenyltransferase |
| D86978 | C7orf14 | 0.53 | C7orf14 | chromosome 7 open reading frame 14 |
| NM_000291 | PGK1 | 0.53 | PGK1 | phosphoglycerate kinase 1 |
| AF016495 | AQP9 | 0.53 | AQP9 | aquaporin 9 |
| AL133017 | FLJ22865 | 0.53 | FLJ22865 | hypothetical protein FLJ22865 |
| Contig45816_RC | C13orf3 | 0.52 | C13orf3 | chromosome 13 open reading frame 3 |
| AK001166 | FLJ11252 | 0.52 | XTP1 | HBxAg transactivated protein 1 |
| NM_001071 | TYMS | 0.52 | TYMS | thymidylate synthetase |
| NM_018131 | C10orf3 | 0.52 | C10orf3 | chromosome 10 open reading frame 3 |
| AF025441 | OIP5 | 0.52 | OIP5 | Opa-interacting protein 5 |
| NM_001211 | BUB1B | 0.52 | BUB1B | BUB1 budding uninhibited by |
| | | | | benzimidazoles 1 homolog beta (yeast) |
| NM_006845 | KIF2C | 0.52 | KIF2C | kinesin family member 2C |
| NM_001762 | CCT6A | 0.52 | CCT6A | chaperonin containing TCP1, subunit 6A |
| | | | | (zeta 1) |
| Contig49270_RC | KIAA1553 | 0.52 | KIAA1553 | KIAA1553 |
| NM_012412 | H2AV | 0.52 | H2AV | histone H2A.F/Z variant |
| Contig54895_RC | FLJ12691 | 0.52 | GALNT14 | UDP-N-acetyl-alpha-D- |
| | | | | galactosamine:polypeptide N- |
| | | | | acetylgalactosaminyltransferase 14 |
| | | | | (GalNAc-T14) |
| NM_004527 | MEOX1 | −0.52 | MEOX1 | mesenchyme homeo box 1 |
| AL137698 | DKFZp434C1915 | −0.53 | PGM5 | phosphoglucomutase 5 |
| Contig52543_RC | MGC29761 | −0.53 | MGC29761 | hypothetical protein MGC29761 |
| Contig37540 | | −0.53 | | Homo sapiens transcribed sequence with |
| | | | | weak similarity to protein |
| | | | | ref: NP_009056.1 (H. sapiens) |
| | | | | ubiquitously transcribed tetratricopeptide |
| | | | | repeat gene, Y chromosome; |
| | | | | Ubiquitously transcribed TPR gene on Y |
| | | | | chromosome [Homo sapiens] |
| NM_004615 | TM4SF2 | −0.53 | TM4SF2 | transmembrane 4 superfamily member 2 |
| NM_002001 | FCER1A | −0.54 | FCER1A | Fc fragment of IgE, high affinity I, |
| | | | | receptor for; alpha polypeptide |
| NM_003551 | NME5 | −0.54 | NME5 | non-metastatic cells 5, protein expressed |
| | | | | in (nucleoside-diphosphate kinase) |
| NM_003152 | STAT5A | −0.55 | STAT5A | signal transducer and activator of |
| | | | | transcription 5A |
| NM_015987 | HEBP1 | −0.55 | HEBP1 | heme binding protein 1 |
| NM_004887 | CXCL14 | −0.55 | CXCL14 | chemokine (C—X—C motif) ligand 14 |
| NM_014773 | KIAA0141 | −0.56 | KIAA0141 | KIAA0141 gene product |

TABLE 5
200 prognosis markers identified by the “Nature method” previously
described (Van't Veer et al. Nature 415(6871): 530-536 (2002)) in
sporadic, ER+ individuals.
Table 5.

| Accession/ | |
|---|---|
| Contig No. | SEQ ID NO.: |
| AB033058 | SEQ ID NO 6 |
| AB033090 | SEQ ID NO 7 |
| AB037734 | SEQ ID NO 9 |
| AB040912 | SEQ ID NO 11 |
| AF007872 | SEQ ID NO 12 |
| AF052117 | SEQ ID NO 15 |
| AF052162 | SEQ ID NO 16 |
| AF064200 | SEQ ID NO 17 |
| AF080158 | SEQ ID NO 19 |
| AF119666 | SEQ ID NO 20 |
| AF131817 | SEQ ID NO 22 |
| AK001560 | SEQ ID NO 26 |
| AK002117 | SEQ ID NO 27 |
| AL049229 | SEQ ID NO 28 |
| AL049685 | SEQ ID NO 29 |
| AL080065 | SEQ ID NO 33 |
| AL080169 | SEQ ID NO 34 |
| AL080218 | SEQ ID NO 36 |
| AL109696 | SEQ ID NO 37 |
| AL110280 | SEQ ID NO 38 |
| AL117544 | SEQ ID NO 39 |
| AL117629 | SEQ ID NO 40 |
| AL122091 | SEQ ID NO 41 |
| AL133017 | SEQ ID NO 42 |
| AL133052 | SEQ ID NO 43 |
| AL137379 | SEQ ID NO 45 |
| D00174 | SEQ ID NO 48 |
| D14678 | SEQ ID NO 49 |
| D25304 | SEQ ID NO 50 |
| D55716 | SEQ ID NO 54 |
| D86980 | SEQ ID NO 56 |
| L19778 | SEQ ID NO 57 |
| L25080 | SEQ ID NO 58 |
| NM_000014 | SEQ ID NO 61 |
| NM_000076 | SEQ ID NO 62 |
| NM_000109 | SEQ ID NO 63 |
| NM_000114 | SEQ ID NO 64 |
| NM_000150 | SEQ ID NO 66 |
| NM_000331 | SEQ ID NO 68 |
| NM_000587 | SEQ ID NO 69 |
| NM_000693 | SEQ ID NO 70 |
| NM_000820 | SEQ ID NO 72 |
| NM_000903 | SEQ ID NO 74 |
| NM_000927 | SEQ ID NO 76 |
| NM_001034 | SEQ ID NO 78 |
| NM_001232 | SEQ ID NO 82 |
| NM_001463 | SEQ ID NO 86 |
| NM_001664 | SEQ ID NO 88 |
| NM_001723 | SEQ ID NO 90 |
| NM_001765 | SEQ ID NO 92 |
| NM_001801 | SEQ ID NO 93 |
| NM_001813 | SEQ ID NO 95 |
| NM_001830 | SEQ ID NO 96 |
| NM_002001 | SEQ ID NO 97 |
| NM_002036 | SEQ ID NO 98 |
| NM_002101 | SEQ ID NO 99 |
| NM_002125 | SEQ ID NO 100 |
| NM_002142 | SEQ ID NO 101 |
| NM_002405 | SEQ ID NO 103 |
| NM_002875 | SEQ ID NO 108 |
| NM_002996 | SEQ ID NO 109 |
| NM_003005 | SEQ ID NO 110 |
| NM_003012 | SEQ ID NO 111 |
| NM_003152 | SEQ ID NO 113 |
| NM_003195 | SEQ ID NO 116 |
| NM_003364 | SEQ ID NO 119 |
| NM_003504 | SEQ ID NO 120 |
| NM_003579 | SEQ ID NO 123 |
| NM_003626 | SEQ ID NO 125 |
| NM_003722 | SEQ ID NO 127 |
| NM_003824 | SEQ ID NO 128 |
| NM_003862 | SEQ ID NO 129 |
| NM_003864 | SEQ ID NO 130 |
| NM_003956 | SEQ ID NO 131 |
| NM_003970 | SEQ ID NO 132 |
| NM_004162 | SEQ ID NO 135 |
| NM_004203 | SEQ ID NO 136 |
| NM_004217 | SEQ ID NO 138 |
| NM_004349 | SEQ ID NO 140 |
| NM_004527 | SEQ ID NO 143 |
| NM_004615 | SEQ ID NO 145 |
| NM_004701 | SEQ ID NO 148 |
| NM_004787 | SEQ ID NO 150 |
| NM_004867 | SEQ ID NO 153 |
| NM_004887 | SEQ ID NO 154 |
| NM_004944 | SEQ ID NO 155 |
| NM_004981 | SEQ ID NO 156 |
| NM_005192 | SEQ ID NO 158 |
| NM_005231 | SEQ ID NO 159 |
| NM_005269 | SEQ ID NO 160 |
| NM_005542 | SEQ ID NO 162 |
| NM_005573 | SEQ ID NO 163 |
| NM_005733 | SEQ ID NO 165 |
| NM_005792 | SEQ ID NO 166 |
| NM_006070 | SEQ ID NO 170 |
| NM_006274 | SEQ ID NO 172 |
| NM_006344 | SEQ ID NO 173 |
| NM_006441 | SEQ ID NO 174 |
| NM_006614 | SEQ ID NO 176 |
| NM_006749 | SEQ ID NO 178 |
| NM_006829 | SEQ ID NO 180 |
| NM_006983 | SEQ ID NO 182 |
| NM_007210 | SEQ ID NO 185 |
| NM_007370 | SEQ ID NO 186 |
| NM_012111 | SEQ ID NO 188 |
| NM_012291 | SEQ ID NO 189 |
| NM_012450 | SEQ ID NO 193 |
| NM_012453 | SEQ ID NO 194 |
| NM_012464 | SEQ ID NO 195 |
| NM_013261 | SEQ ID NO 197 |
| NM_013272 | SEQ ID NO 198 |
| NM_013277 | SEQ ID NO 199 |
| NM_013981 | SEQ ID NO 200 |
| NM_013999 | SEQ ID NO 201 |
| NM_014015 | SEQ ID NO 202 |
| NM_014067 | SEQ ID NO 203 |
| NM_014258 | SEQ ID NO 206 |
| NM_014737 | SEQ ID NO 211 |
| NM_014791 | SEQ ID NO 213 |
| NM_014926 | SEQ ID NO 214 |
| NM_015544 | SEQ ID NO 215 |
| NM_016049 | SEQ ID NO 220 |
| NM_016250 | SEQ ID NO 222 |
| NM_016442 | SEQ ID NO 224 |
| NM_016815 | SEQ ID NO 226 |
| NM_017647 | SEQ ID NO 228 |
| NM_017681 | SEQ ID NO 229 |
| NM_017888 | SEQ ID NO 232 |
| NM_017899 | SEQ ID NO 233 |
| NM_018014 | SEQ ID NO 235 |
| NM_018154 | SEQ ID NO 239 |
| NM_018286 | SEQ ID NO 240 |
| NM_018335 | SEQ ID NO 241 |
| NM_018390 | SEQ ID NO 242 |
| NM_018659 | SEQ ID NO 247 |
| NM_019013 | SEQ ID NO 249 |
| NM_021052 | SEQ ID NO 254 |
| NM_021064 | SEQ ID NO 255 |
| NM_021183 | SEQ ID NO 257 |
| U56387 | SEQ ID NO 261 |
| V00522 | SEQ ID NO 265 |
| X74794 | SEQ ID NO 266 |
| AA598803_RC | SEQ ID NO 268 |
| AW190932_RC | SEQ ID NO 270 |
| NM_018692 | SEQ ID NO 273 |
| Contig2313_RC | SEQ ID NO 276 |
| Contig8113_RC | SEQ ID NO 279 |
| Contig8373_RC | SEQ ID NO 280 |
| Contig37540 | SEQ ID NO 286 |
| Contig49875 | SEQ ID NO 288 |
| Contig50368 | SEQ ID NO 289 |
| Contig10007_RC | SEQ ID NO 292 |
| Contig18296_RC | SEQ ID NO 296 |
| Contig18543_RC | SEQ ID NO 298 |
| Contig20512_RC | SEQ ID NO 299 |
| Contig21986_RC | SEQ ID NO 300 |
| Contig22337_RC | SEQ ID NO 301 |
| Contig23475_RC | SEQ ID NO 302 |
| Contig25938_RC | SEQ ID NO 304 |
| Contig26022_RC | SEQ ID NO 305 |
| Contig26059_RC | SEQ ID NO 306 |
| Contig26492_RC | SEQ ID NO 307 |
| Contig27623_RC | SEQ ID NO 308 |
| Contig30993_RC | SEQ ID NO 312 |
| Contig31361_RC | SEQ ID NO 313 |
| Contig32604_RC | SEQ ID NO 316 |
| Contig34430_RC | SEQ ID NO 320 |
| Contig36243_RC | SEQ ID NO 324 |
| Contig37198_RC | SEQ ID NO 325 |
| Contig38463_RC | SEQ ID NO 327 |
| Contig40552_RC | SEQ ID NO 332 |
| Contig41413_RC | SEQ ID NO 333 |
| Contig41990_RC | SEQ ID NO 334 |
| Contig42036_RC | SEQ ID NO 335 |
| Contig42103_RC | SEQ ID NO 336 |
| Contig43289_RC | SEQ ID NO 337 |
| Contig43648_RC | SEQ ID NO 338 |
| Contig43927_RC | SEQ ID NO 342 |
| Contig45441_RC | SEQ ID NO 345 |
| Contig46351_RC | SEQ ID NO 349 |
| Contig46756_RC | SEQ ID NO 350 |
| Contig47308_RC | SEQ ID NO 352 |
| Contig48353_RC | SEQ ID NO 355 |
| Contig49279_RC | SEQ ID NO 357 |
| Contig49773_RC | SEQ ID NO 358 |
| Contig50470_RC | SEQ ID NO 360 |
| Contig50661_RC | SEQ ID NO 361 |
| Contig51456_RC | SEQ ID NO 364 |
| Contig51710_RC | SEQ ID NO 365 |
| Contig52193_RC | SEQ ID NO 366 |
| Contig54895_RC | SEQ ID NO 372 |
| Contig54926_RC | SEQ ID NO 373 |
| Contig54956_RC | SEQ ID NO 374 |
| Contig54993_RC | SEQ ID NO 375 |
| Contig55725_RC | SEQ ID NO 376 |
| Contig56843_RC | SEQ ID NO 380 |
| Contig57803_RC | SEQ ID NO 383 |
| Contig58260_RC | SEQ ID NO 384 |
| Contig59294_RC | SEQ ID NO 385 |
| Contig59870_RC | SEQ ID NO 386 |

TABLE 6
Accession/contig number, gene name, correlation to prognosis, and description for
each of the markers listed in Table 5.
Table 6.

| Accession/ | | | | |
|---|---|---|---|---|
| Contig No. | Gene | Corr | Name | Description |
| AL133017 | FLJ22865 | 0.43 | FLJ22865 | hypothetical protein FLJ22865 |
| AF119666 | LOC55971 | 0.4 | LOC55971 | Homo sapiens insulin receptor tyrosine |
| | | | | kinase substrate mRNA, complete cds. |
| Contig54895_RC | FLJ12691 | 0.38 | GALNT14 | UDP-N-acetyl-alpha-D- |
| | | | | galactosamine:polypeptide N- |
| | | | | acetylgalactosaminyltransferase 14 |
| | | | | (GalNAc-T14) |
| NM_018154 | ASF1B | 0.37 | ASF1B | ASF1 anti-silencing function 1 homolog B |
| | | | | (S. cerevisiae) |
| Contig18543_RC | | 0.37 | | Homo sapiens mRNA; cDNA |
| | | | | DKFZp686F1782 (from clone |
| | | | | DKFZp686F1782) |
| NM_003195 | TCEA2 | 0.37 | TCEA2 | transcription elongation factor A (SII), 2 |
| NM_005573 | LMNB1 | 0.36 | LMNB1 | lamin B1 |
| AA598803_RC | | 0.36 | LOC139886 | hypothetical protein LOC139886 |
| AL049685 | RAP2C | 0.36 | RAP2C | RAP2C, member of RAS oncogene |
| | | | | family |
| L19778 | HIST1H2AG | 0.36 | HIST1H2AG | histone 1, H2ag |
| NM_003626 | PPFIA1 | 0.36 | PPFIA1 | protein tyrosine phosphatase, receptor |
| | | | | type, f polypeptide (PTPRF), interacting |
| | | | | protein (liprin), alpha 1 |
| NM_018335 | C14orf131 | 0.36 | C14orf131 | chromosome 14 open reading frame 131 |
| NM_004217 | STK12 | 0.35 | AURKB | aurora kinase B |
| NM_013277 | RACGAP1 | 0.35 | RACGAP1 | Rac GTPase activating protein 1 |
| NM_021183 | RAP2C | 0.35 | RAP2C | RAP2C, member of RAS oncogene |
| | | | | family |
| AF052162 | FLJ12443 | 0.35 | FLJ12443 | hypothetical protein FLJ12443 |
| NM_000903 | NQO1 | 0.35 | NQO1 | NAD(P)H dehydrogenase, quinone 1 |
| NM_001034 | RRM2 | 0.34 | RRM2 | ribonucleotide reductase M2 polypeptide |
| NM_021064 | HIST1H2AG | 0.34 | HIST1H2AG | histone 1, H2ag |
| AL137379 | FLJ13912 | 0.34 | FLJ13912 | hypothetical protein FLJ13912 |
| NM_014258 | SYCP2 | 0.34 | SYCP2 | synaptonemal complex protein 2 |
| AF007872 | TOR1B | 0.34 | TOR1B | torsin family 1, member B (torsin B) |
| NM_001664 | ARHA | 0.34 | ARHA | ras homolog gene family, member A |
| AF064200 | UGT2B4 | 0.34 | UGT2B4 | UDP glycosyltransferase 2 family, |
| | | | | polypeptide B4 |
| Contig36243_RC | | 0.34 | DKFZP434A1022 | hypothetical protein DKFZp434A1022 |
| D55716 | MCM7 | 0.33 | MCM7 | MCM7 minichromosome maintenance |
| | | | | deficient 7 (S. cerevisiae) |
| Contig22337_RC | OXR1 | 0.33 | OXR1 | oxidation resistance 1 |
| NM_001813 | CENPE | 0.33 | CENPE | centromere protein E, 312 kDa |
| NM_012111 | AHSA1 | 0.33 | AHSA1 | AHA1, activator of heat shock 90 kDa |
| | | | | protein ATPase homolog 1 (yeast) |
| AL049229 | | 0.33 | | Homo sapiens mRNA; cDNA |
| | | | | DKFZp564O1016 (from clone |
| | | | | DKFZp564O1016) |
| AK002117 | | 0.33 | GNA13 | guanine nucleotide binding protein (G |
| | | | | protein), alpha 13 |
| Contig31361_RC | | 0.33 | DOCK7 | dedicator of cytokinesis 7 |
| Contig40552_RC | FLJ25348 | 0.33 | FLJ25348 | hypothetical protein FLJ25348 |
| NM_003364 | UP | 0.33 | UPP1 | uridine phosphorylase 1 |
| NM_012450 | SLC13A4 | 0.33 | SLC13A4 | solute carrier family 13 (sodium/sulfate |
| | | | | symporters), member 4 |
| NM_014067 | LRP16 | 0.33 | LRP16 | LRP16 protein |
| NM_002875 | RAD51 | 0.32 | RAD51 | RAD51 homolog (RecA homolog, E. coli) |
| | | | | (S. cerevisiae) |
| D14678 | KIFC1 | 0.32 | KIFC1 | kinesin family member C1 |
| NM_005733 | KIF20A | 0.32 | KIF20A | kinesin family member 20A |
| NM_005231 | EMS1 | 0.32 | EMS1 | ems1 sequence (mammary tumor and |
| | | | | squamous cell carcinoma-associated |
| | | | | (p80/85 src substrate) |
| NM_003864 | SAP30 | 0.32 | SAP30 | sin3-associated polypeptide, 30 kDa |
| AW190932_RC | GPI | 0.32 | GPI | xl66g09.x1 NCI_CGAP_Pan1 Homo |
| | | | | sapiens cDNA clone IMAGE: 2679712 3′, |
| | | | | mRNA sequence. |
| Contig23475_RC | | 0.32 | FLJ23471 | MICAL-like 2 |
| L25080 | ARHA | 0.32 | ARHA | ras homolog gene family, member A |
| NM_007210 | GALNT6 | 0.32 | GALNT6 | UDP-N-acetyl-alpha-D- |
| | | | | galactosamine:polypeptide N- |
| | | | | acetylgalactosaminyltransferase 6 |
| | | | | (GalNAc-T6) |
| D86980 | KIAA0227 | 0.32 | TTC9 | tetratricopeptide repeat domain 9 |
| NM_003579 | RAD54L | 0.31 | RAD54L | RAD54-like (S. cerevisiae) |
| Contig41413_RC | RRM2 | 0.31 | RRM2 | ribonucleotide reductase M2 polypeptide |
| Contig48353_RC | FKSG14 | 0.31 | FKSG14 | leucine zipper protein FKSG14 |
| NM_004162 | RAB5A | 0.31 | RAB5A | RAB5A, member RAS oncogene family |
| NM_006070 | TFG | 0.31 | TFG | TRK-fused gene |
| Contig34430_RC | FLJ10656 | 0.31 | P15RS | hypothetical protein FLJ10656 |
| AB033058 | DLG3 | 0.31 | DLG3 | discs, large homolog 3 (neuroendocrine- |
| | | | | dlg, Drosophila) |
| NM_016442 | ARTS-1 | 0.31 | ARTS-1 | type 1 tumor necrosis factor receptor |
| | | | | shedding aminopeptidase regulator |
| Contig51456_RC | LOC93109 | 0.31 | LOC93109 | hypothetical protein BC007772 |
| NM_005542 | INSIG1 | 0.31 | INSIG1 | insulin induced gene 1 |
| AL133052 | C1orf37 | 0.31 | C1orf37 | chromosome 1 open reading frame 37 |
| Contig54926_RC | SFTPC | 0.31 | SFTPC | Homo sapiens cDNA FLJ42627 fis, clone |
| | | | | BRACE3018308 |
| NM_021052 | HIST1H2AE | 0.31 | HIST1H2AE | histone 1, H2ae |
| NM_003504 | CDC45L | 0.3 | CDC45L | CDC45 cell division cycle 45-like (S. cerevisiae) |
| Contig56843_RC | CCNB1 | 0.3 | CCNB1 | cyclin B1 |
| NM_019013 | FLJ10156 | 0.3 | FLJ10156 | hypothetical protein FLJ10156 |
| NM_012291 | ESPL1 | 0.3 | ESPL1 | extra spindle poles like 1 (S. cerevisiae) |
| NM_007370 | RFC5 | 0.3 | RFC5 | replication factor C (activator 1) 5, |
| | | | | 36.5 kDa |
| NM_014791 | MELK | 0.3 | MELK | maternal embryonic leucine zipper kinase |
| NM_005192 | CDKN3 | 0.3 | CDKN3 | cyclin-dependent kinase inhibitor 3 |
| | | | | (CDK2-associated dual specificity |
| | | | | phosphatase) |
| X74794 | MCM4 | 0.3 | MCM4 | MCM4 minichromosome maintenance |
| | | | | deficient 4 (S. cerevisiae) |
| NM_018390 | FLJ11323 | 0.3 | FLJ11323 | hypothetical protein FLJ11323 |
| NM_004203 | RBL2 | 0.3 | PKMYT1 | membrane-associated tyrosine- and |
| | | | | threonine-specific cdc2-inhibitory kinase |
| NM_016049 | LOC51016 | 0.3 | C14orf122 | chromosome 14 open reading frame 122 |
| NM_012453 | TBL2 | 0.3 | TBL2 | transducin (beta)-like 2 |
| NM_000150 | FUT6 | 0.3 | FUT6 | fucosyltransferase 6 (alpha (1,3) |
| | | | | fucosyltransferase) |
| Contig18296_RC | | 0.3 | | Homo sapiens transcribed sequences |
| NM_006441 | MTHFS | 0.3 | MTHFS | 5,10-methenyltetrahydrofolate synthetase |
| | | | | (5-formyltetrahydrofolate cyclo-ligase) |
| Contig21986_RC | | 0.3 | | Homo sapiens transcribed sequence with |
| | | | | moderate similarity to protein |
| | | | | ref: NP_112198.1 (H. sapiens) ring finger |
| | | | | protein 32 [Homo sapiens] |
| NM_004981 | KCNJ4 | 0.3 | KCNJ4 | potassium inwardly-rectifying channel, |
| | | | | subfamily J, member 4 |
| NM_003824 | FADD | 0.3 | FADD | Fas (TNFRSF6)-associated via death |
| | | | | domain |
| AF080158 | IKBKB | 0.3 | IKBKB | inhibitor of kappa light polypeptide gene |
| | | | | enhancer in B-cells, kinase beta |
| NM_017681 | FLJ20130 | 0.3 | FLJ20130 | hypothetical protein FLJ20130 |
| NM_004701 | CCNB2 | 0.29 | CCNB2 | cyclin B2 |
| Contig55725_RC | CDCA7 | 0.29 | CDCA7 | cell division cycle associated 7 |
| NM_005792 | MPHOSPH6 | 0.29 | MPHOSPH6 | M-phase phosphoprotein 6 |
| NM_017647 | FTSJ3 | 0.29 | FTSJ3 | FtsJ homolog 3 (E. coli) |
| AL122091 | LOC56965 | 0.29 | LOC56965 | hypothetical protein from EUROIMAGE |
| | | | | 1977056 |
| AL117629 | DKFZP434C245 | 0.29 | DKFZP434C245 | DKFZP434C245 protein |
| Contig26059_RC | | 0.29 | | Homo sapiens transcribed sequences |
| Contig43927_RC | | 0.29 | | Homo sapiens transcribed sequences |
| NM_006749 | SLC20A2 | 0.29 | SLC20A2 | solute carrier family 20 (phosphate |
| | | | | transporter), member 2 |
| Contig51710_RC | | −0.29 | | Homo sapiens clone DNA100312 |
| | | | | VSSW1971 (UNQ1971) mRNA, complete |
| | | | | cds |
| NM_018014 | BCL11A | −0.29 | BCL11A | B-cell CLL/lymphoma 11A (zinc finger |
| | | | | protein) |
| Contig26492_RC | C12orf6 | −0.29 | C12orf6 | chromosome 12 open reading frame 6 |
| Contig32604_RC | | −0.29 | FLJ11753 | hypothetical protein FLJ11753 |
| NM_001801 | CDO1 | −0.29 | CDO1 | cysteine dioxygenase, type I |
| NM_006614 | CHL1 | −0.29 | CHL1 | cell adhesion molecule with homology to |
| | | | | L1CAM (close homolog of L1) |
| Contig45441_RC | | −0.29 | LOC284542 | hypothetical protein LOC284542 |
| D25304 | ARHGEF6 | −0.29 | ARHGEF6 | Rac/Cdc42 guanine nucleotide exchange |
| | | | | factor (GEF) 6 |
| NM_006983 | MMP23B | −0.29 | MMP23B | matrix metalloproteinase 23B |
| Contig38463_RC | LOC64150 | −0.29 | C14orf134 | deiodinase, iodothyronine, type III |
| | | | | opposite strand |
| NM_003005 | SELP | −0.29 | SELP | selectin P (granule membrane protein |
| | | | | 140 kDa, antigen CD62) |
| NM_014926 | KIAA0848 | −0.29 | KIAA0848 | KIAA0848 protein |
| AL080169 | DKFZP434C171 | −0.3 | DKFZP434C171 | DKFZP434C171 protein |
| Contig43289_RC | | −0.3 | LOC170371 | hypothetical protein LOC170371 |
| AK001560 | NECL1 | −0.3 | NECL1 | nectin-like protein 1 |
| NM_006274 | CCL19 | −0.3 | CCL19 | chemokine (C-C motif) ligand 19 |
| NM_006344 | HML2 | −0.3 | CLECSF13 | C-type (calcium dependent, |
| | | | | carbohydrate-recognition domain) lectin, |
| | | | | superfamily member 13 (macrophage- |
| | | | | derived) |
| D00174 | SERPINF2 | −0.3 | SERPINF2 | serine (or cysteine) proteinase inhibitor, |
| | | | | clade F (alpha-2 antiplasmin, pigment |
| | | | | epithelium derived factor), member 2 |
| Contig10007_RC | | −0.3 | | Homo sapiens similar to MHC HLA-SX- |
| | | | | alpha (LOC377373), mRNA |
| Contig43648_RC | | −0.3 | | Homo sapiens transcribed sequences |
| Contig54956_RC | FLJ23119 | −0.3 | FLJ23119 | hypothetical protein FLJ23119 |
| NM_000076 | CDKN1C | −0.3 | CDKN1C | cyclin-dependent kinase inhibitor 1C |
| | | | | (p57, Kip2) |
| NM_000927 | ABCB1 | −0.3 | ABCB1 | ATP-binding cassette, sub-family B |
| | | | | (MDR/TAP), member 1 |
| Contig41990_RC | DKFZP434D0127 | −0.3 | USP44 | ubiquitin specific protease 44 |
| NM_001723 | BPAG1 | −0.3 | BPAG1 | bullous pemphigoid antigen 1, |
| | | | | 230/240 kDa |
| Contig50661_RC | FLJ14440 | −0.3 | THSD2 | thrombospondin, type I, domain 2 |
| AF052117 | | −0.3 | CLCN4 | chloride channel 4 |
| Contig8373_RC | | −0.3 | | Homo sapiens transcribed sequence with |
| | | | | weak similarity to protein |
| | | | | ref: NP_060312.1 (H. sapiens) |
| | | | | hypothetical protein FLJ20489 |
| | | | | [Homo sapiens] |
| AL109696 | | −0.3 | | Homo sapiens mRNA full length insert |
| | | | | cDNA clone EUROIMAGE 21920 |
| Contig25938_RC | | −0.3 | EBF2 | early B-cell factor 2 |
| AB040912 | SEMA6D | −0.3 | SEMA6D | sema domain, transmembrane domain |
| | | | | (TM), and cytoplasmic domain, |
| | | | | (semaphorin) 6D |
| Contig57803_RC | CHD1L | −0.3 | CHD1L | chromodomain helicase DNA binding |
| | | | | protein 1-like |
| AB037734 | PCDH19 | −0.3 | PCDH19 | protocadherin 19 |
| NM_004787 | SLIT2 | −0.3 | SLIT2 | slit homolog 2 (Drosophila) |
| NM_001232 | CASQ2 | −0.3 | CASQ2 | calsequestrin 2 (cardiac muscle) |
| Contig2313_RC | | −0.3 | DKFZp667B0210 | hypothetical protein DKFZp667B0210 |
| AL080218 | | −0.3 | STAT5B | signal transducer and activator of |
| | | | | transcription 5B |
| AL080065 | DKFZP564J102 | −0.3 | DKFZP564J102 | DKFZP564J102 protein |
| NM_002142 | HOXA9 | −0.3 | HOXA9 | homeo box A9 |
| Contig59294_RC | EPC1 | −0.3 | EPC1 | enhancer of polycomb homolog 1, |
| | | | | (Drosophila) |
| AL117544 | DKFZP434I092 | −0.3 | DKFZP434I092 | DKFZP434I092 protein |
| NM_002101 | GYPC | −0.31 | GYPC | glycophorin C (Gerbich blood group) |
| NM_002405 | MFNG | −0.31 | MFNG | manic fringe homolog (Drosophila) |
| Contig52193_RC | | −0.31 | ABCB1 | ATP-binding cassette, sub-family B |
| | | | | (MDR/TAP), member 1 |
| Contig58260_RC | DGAT2 | −0.31 | DGAT2 | diacylglycerol O-acyltransferase homolog |
| | | | | 2 (mouse) |
| Contig49773_RC | CYYR1 | −0.31 | CYYR1 | cysteine and tyrosine-rich 1 |
| Contig54993_RC | CLDN11 | −0.31 | CLDN11 | claudin 11 (oligodendrocyte |
| | | | | transmembrane protein) |
| AB033090 | PAK7 | −0.31 | PAK7 | p21(CDKN1A)-activated kinase 7 |
| NM_003722 | TP73L | −0.31 | TP73L | tumor protein p73-like |
| NM_002996 | CX3CL1 | −0.31 | CX3CL1 | chemokine (C—X3—C motif) ligand 1 |
| Contig30993_RC | BACH2 | −0.31 | BACH2 | BTB and CNC homology 1, basic leucine |
| | | | | zipper transcription factor 2 |
| NM_000693 | ALDH1A3 | −0.31 | ALDH1A3 | aldehyde dehydrogenase 1 family, |
| | | | | member A3 |
| NM_014737 | RASSF2 | −0.31 | RASSF2 | Ras association (RaIGDS/AF-6) domain |
| | | | | family 2 |
| Contig46351_RC | | −0.31 | | Homo sapiens transcribed sequence with |
| | | | | weak similarity to protein pir: JC1405 |
| | | | | (H. sapiens) JC1405 6- |
| | | | | pyruvoyltetrahydropterin synthase- |
| | | | | human |
| Contig8113_RC | | −0.31 | | Homo sapiens transcribed sequences |
| NM_002125 | HLA-DRB5 | −0.31 | HLA-DRB5 | major histocompatibility complex, class II, |
| | | | | DR beta 3 |
| Contig37198_RC | HSPA12B | −0.31 | HSPA12B | heat shock 70 kD protein 12B |
| AF131817 | | −0.31 | CBFA2T1 | core-binding factor, runt domain, alpha |
| | | | | subunit 2; translocated to, 1; cyclin D- |
| | | | | related |
| NM_018659 | C17 | −0.31 | C17 | cytokine-like protein C17 |
| Contig20512_RC | | −0.31 | LOC285671 | hypothetical protein LOC285671 |
| NM_014015 | MYLE | −0.31 | DEXI | dexamethasone-induced transcript |
| NM_001765 | CD1C | −0.32 | CD1C | CD1C antigen, c polypeptide |
| NM_018692 | C20orf17 | −0.32 | C20orf17 | chromosome 20 open reading frame 17 |
| Contig49875 | | −0.32 | | Homo sapiens full length insert cDNA |
| | | | | YN61C04 |
| Contig59870_RC | IRX1 | −0.32 | IRX1 | iroquois homeobox protein 1 |
| Contig42036_RC | BACH2 | −0.32 | BACH2 | BTB and CNC homology 1, basic leucine |
| | | | | zipper transcription factor 2 |
| Contig46756_RC | SYN2 | −0.32 | SYN2 | synapsin II |
| Contig50470_RC | KIAA1921 | −0.32 | KIAA1921 | KIAA1921 protein |
| NM_013272 | SLC21A11 | −0.32 | SLC21A11 | solute carrier organic anion transporter |
| | | | | family, member 3A1 |
| NM_000820 | GAS6 | −0.32 | GAS6 | growth arrest-specific 6 |
| Contig27623_RC | | −0.32 | | Homo sapiens transcribed sequences |
| NM_016250 | NDRG2 | −0.32 | NDRG2 | NDRG family member 2 |
| NM_006829 | APM2 | −0.32 | APM2 | adipose specific 2 |
| NM_015544 | DKFZP564K1964 | −0.32 | DKFZP564K1964 | DKFZP564K1964 protein |
| NM_016815 | GYPC | −0.33 | GYPC | glycophorin C (Gerbich blood group) |
| NM_000014 | A2M | −0.33 | A2M | alpha-2-macroglobulin |
| NM_004887 | CXCL14 | −0.33 | CXCL14 | chemokine (C—X—C motif) ligand 14 |
| U56387 | PCSK5 | −0.33 | PCSK5 | proprotein convertase subtilisin/kexin |
| | | | | type 5 |
| NM_018286 | FLJ10970 | −0.34 | FLJ10970 | hypothetical protein FLJ10970 |
| NM_002001 | FCER1A | −0.34 | FCER1A | Fc fragment of IgE, high affinity I, |
| | | | | receptor for; alpha polypeptide |
| NM_003012 | SFRP1 | −0.34 | SFRP1 | secreted frizzled-related protein 1 |
| NM_000114 | EDN3 | −0.34 | EDN3 | endothelin 3 |
| NM_000331 | SAA1 | −0.34 | SAA1 | serum amyloid A1 |
| NM_003862 | FGF18 | −0.34 | FGF18 | fibroblast growth factor 18 |
| NM_004349 | CBFA2T1 | −0.34 | CBFA2T1 | core-binding factor, runt domain, alpha |
| | | | | subunit 2; translocated to, 1; cyclin D- |
| | | | | related |
| NM_005269 | GLI | −0.34 | GLI | glioma-associated oncogene homolog |
| | | | | (zinc finger protein) |
| Contig50368 | | −0.34 | | cDNA encoding novel polypeptide from |
| | | | | human umbilical vein endothelial cell. |
| NM_017888 | FLJ20581 | −0.34 | FLJ20581 | hypothetical protein FLJ20581 |
| Contig42103_RC | | −0.35 | C20orf17 | chromosome 20 open reading frame 17 |
| Contig49279_RC | | −0.35 | FLJ25461 | hypothetical protein FLJ25461 |
| NM_001830 | CLCN4 | −0.35 | CLCN4 | chloride channel 4 |
| NM_001463 | FRZB | −0.35 | FRZB | frizzled-related protein |
| NM_003970 | MYOM2 | −0.35 | MYOM2 | myomesin (M-protein) 2, 165 kDa |
| V00522 | HLA-DRB3 | −0.35 | HLA-DRB3 | major histocompatibility complex, class II, |
| | | | | DR beta 3 |
| NM_013999 | MEOX1 | −0.36 | MEOX1 | mesenchyme homeo box 1 |
| NM_000587 | C7 | −0.36 | C7 | complement component 7 |
| AL110280 | | −0.36 | PAPLN | papilin, proteoglycan-like sulfated |
| | | | | glycoprotein |
| NM_000109 | DMD | −0.36 | DMD | dystrophin (muscular dystrophy, |
| | | | | Duchenne and Becker types) |
| Contig26022_RC | MGC13057 | −0.36 | MGC13057 | hypothetical protein MGC13057 |
| NM_013981 | NRG2 | −0.36 | NRG2 | neuregulin 2 |
| NM_013261 | PPARGC1 | −0.36 | PPARGC1 | peroxisome proliferative activated |
| | | | | receptor, gamma, coactivator 1 |
| Contig37540 | | −0.37 | | Homo sapiens transcribed sequence with |
| | | | | weak similarity to protein |
| | | | | ref: NP_009056.1 (H. sapiens) |
| | | | | ubiquitously transcribed tetratricopeptide |
| | | | | repeat gene, Y chromosome; |
| | | | | Ubiquitously transcribed TPR gene on Y |
| | | | | chromosome [Homo sapiens] |
| NM_017899 | TSC | −0.37 | TSC | hypothetical protein FLJ20607 |
| NM_003152 | STAT5A | −0.37 | STAT5A | signal transducer and activator of |
| | | | | transcription 5A |
| NM_002036 | FY | −0.38 | FY | Duffy blood group |
| NM_004867 | ITM2A | −0.38 | ITM2A | integral membrane protein 2A |
| Contig47308_RC | | −0.38 | | Homo sapiens hypothetical gene |
| | | | | supported by NM_018692 (LOC374296), |
| | | | | mRNA |
| NM_004527 | MEOX1 | −0.39 | MEOX1 | mesenchyme homeo box 1 |
| NM_004615 | TM4SF2 | −0.4 | TM4SF2 | transmembrane 4 superfamily member 2 |
| NM_003956 | CH25H | −0.4 | CH25H | cholesterol 25-hydroxylase |
| NM_012464 | TLL1 | −0.4 | TLL1 | tolloid-like 1 |
| NM_004944 | DNASE1L3 | −0.41 | DNASE1L3 | deoxyribonuclease I-like 3 |

TABLE 7
100 prognosis markers identified by an iterative
method in sporadic, ER+ individuals.
Table 7.

| Accession/ | |
|---|---|
| Contig No. | SEQ ID NO.: |
| AB024704 | SEQ ID NO 2 |
| AF052162 | SEQ ID NO 16 |
| AF119666 | SEQ ID NO 20 |
| AF131817 | SEQ ID NO 22 |
| AK001166 | SEQ ID NO 25 |
| AK001560 | SEQ ID NO 26 |
| AK002117 | SEQ ID NO 27 |
| AL049685 | SEQ ID NO 29 |
| AL049949 | SEQ ID NO 30 |
| AL080169 | SEQ ID NO 34 |
| AL110280 | SEQ ID NO 38 |
| AL133017 | SEQ ID NO 42 |
| AL137698 | SEQ ID NO 47 |
| D00174 | SEQ ID NO 48 |
| D55716 | SEQ ID NO 54 |
| NM_000076 | SEQ ID NO 62 |
| NM_000109 | SEQ ID NO 63 |
| NM_000114 | SEQ ID NO 64 |
| NM_000331 | SEQ ID NO 68 |
| NM_000587 | SEQ ID NO 69 |
| NM_000820 | SEQ ID NO 72 |
| NM_000927 | SEQ ID NO 76 |
| NM_001034 | SEQ ID NO 78 |
| NM_001071 | SEQ ID NO 79 |
| NM_001237 | SEQ ID NO 83 |
| NM_001463 | SEQ ID NO 86 |
| NM_002001 | SEQ ID NO 97 |
| NM_002036 | SEQ ID NO 98 |
| NM_002101 | SEQ ID NO 99 |
| NM_002358 | SEQ ID NO 102 |
| NM_002405 | SEQ ID NO 103 |
| NM_002875 | SEQ ID NO 108 |
| NM_002996 | SEQ ID NO 109 |
| NM_003012 | SEQ ID NO 111 |
| NM_003152 | SEQ ID NO 113 |
| NM_003504 | SEQ ID NO 120 |
| NM_003600 | SEQ ID NO 124 |
| NM_003626 | SEQ ID NO 125 |
| NM_003686 | SEQ ID NO 126 |
| NM_003862 | SEQ ID NO 129 |
| NM_003956 | SEQ ID NO 131 |
| NM_003970 | SEQ ID NO 132 |
| NM_003981 | SEQ ID NO 133 |
| NM_004162 | SEQ ID NO 135 |
| NM_004217 | SEQ ID NO 138 |
| NM_004349 | SEQ ID NO 140 |
| NM_004526 | SEQ ID NO 142 |
| NM_004527 | SEQ ID NO 143 |
| NM_004615 | SEQ ID NO 145 |
| NM_004701 | SEQ ID NO 148 |
| NM_004787 | SEQ ID NO 150 |
| NM_004867 | SEQ ID NO 153 |
| NM_004887 | SEQ ID NO 154 |
| NM_004944 | SEQ ID NO 155 |
| NM_005192 | SEQ ID NO 158 |
| NM_005269 | SEQ ID NO 160 |
| NM_005542 | SEQ ID NO 162 |
| NM_005573 | SEQ ID NO 163 |
| NM_005733 | SEQ ID NO 165 |
| NM_006027 | SEQ ID NO 169 |
| NM_012310 | SEQ ID NO 190 |
| NM_012464 | SEQ ID NO 195 |
| NM_013261 | SEQ ID NO 197 |
| NM_013277 | SEQ ID NO 199 |
| NM_013981 | SEQ ID NO 200 |
| NM_013999 | SEQ ID NO 201 |
| NM_014176 | SEQ ID NO 204 |
| NM_014791 | SEQ ID NO 213 |
| NM_016049 | SEQ ID NO 220 |
| NM_016250 | SEQ ID NO 222 |
| NM_016602 | SEQ ID NO 225 |
| NM_016815 | SEQ ID NO 226 |
| NM_018154 | SEQ ID NO 239 |
| NM_018286 | SEQ ID NO 240 |
| NM_018492 | SEQ ID NO 244 |
| NM_020675 | SEQ ID NO 251 |
| U96131 | SEQ ID NO 264 |
| NM_003158 | SEQ ID NO 269 |
| Contig34952 | SEQ ID NO 284 |
| Contig37540 | SEQ ID NO 286 |
| Contig26022_RC | SEQ ID NO 305 |
| Contig27623_RC | SEQ ID NO 308 |
| Contig31646_RC | SEQ ID NO 314 |
| Contig33814_RC | SEQ ID NO 317 |
| Contig37198_RC | SEQ ID NO 325 |
| Contig38901_RC | SEQ ID NO 328 |
| Contig41413_RC | SEQ ID NO 333 |
| Contig42036_RC | SEQ ID NO 335 |
| Contig42103_RC | SEQ ID NO 336 |
| Contig43289_RC | SEQ ID NO 337 |
| Contig43759_RC | SEQ ID NO 340 |
| Contig45821_RC | SEQ ID NO 348 |
| Contig46756_RC | SEQ ID NO 350 |
| Contig46796_RC | SEQ ID NO 351 |
| Contig47308_RC | SEQ ID NO 352 |
| Contig49279_RC | SEQ ID NO 357 |
| Contig49869_RC | SEQ ID NO 359 |
| Contig52419_RC | SEQ ID NO 367 |
| Contig54993_RC | SEQ ID NO 375 |
| Contig59870_RC | SEQ ID NO 386 |

TABLE 8
Accession/contig number, gene name, correlation to prognosis, and description for
each of the markers listed in Table 1.
Table 8.

| Accession/ | | | | |
|---|---|---|---|---|
| Contig No. | Gene | Corr. | Name | Description |
| NM_013277 | RACGAP1 | 0.61 | RACGAP1 | Rac GTPase activating protein 1 |
| NM_003504 | CDC45L | 0.59 | CDC45L | CDC45 cell division cycle 45-like (S. cerevisiae) |
| NM_005573 | LMNB1 | 0.59 | LMNB1 | lamin B1 |
| NM_002358 | MAD2L1 | 0.58 | MAD2L1 | MAD2 mitotic arrest deficient-like 1 (yeast) |
| NM_018492 | TOPK | 0.58 | TOPK | T-LAK cell-originated protein kinase |
| AL133017 | FLJ22865 | 0.58 | FLJ22865 | hypothetical protein FLJ22865 |
| AK001166 | FLJ11252 | 0.57 | XTP1 | HBxAg transactivated protein 1 |
| NM_002875 | RAD51 | 0.56 | RAD51 | RAD51 homolog (RecA homolog, E. coli) (S. cerevisiae) |
| AF119666 | LOC55971 | 0.56 | LOC55971 | Homo sapiens insulin receptor tyrosine |
| | | | | kinase substrate mRNA, complete cds. |
| NM_003158 | STK6 | 0.55 | STK6 | Homo sapiens mRNA for aurora/IPL1- |
| | | | | related kinase, complete cds. |
| NM_012310 | KIF4A | 0.55 | KIF4A | kinesin family member 4A |
| NM_001034 | RRM2 | 0.55 | RRM2 | ribonucleotide reductase M2 polypeptide |
| NM_014176 | HSPC150 | 0.55 | HSPC150 | HSPC150 protein similar to ubiquitin- |
| | | | | conjugating enzyme |
| AK002117 | | 0.55 | GNA13 | |
| NM_020675 | AD024 | 0.54 | AD024 | |
| Contig41413_RC | RRM2 | 0.54 | RRM2 | ribonucleotide reductase M2 polypeptide |
| NM_003686 | EXO1 | 0.54 | EXO1 | exonuclease 1 |
| D55716 | MCM7 | 0.54 | MCM7 | MCM7 minichromosome maintenance |
| | | | | deficient 7 (S. cerevisiae) |
| AB024704 | C20orf1 | 0.53 | C20orf1 | TPX2, microtubule-associated protein |
| | | | | homolog (Xenopus laevis) |
| Contig38901_RC | MGC45866 | 0.53 | MGC45866 | hypothetical protein MGC45866 |
| NM_001237 | CCNA2 | 0.53 | CCNA2 | cyclin A2 |
| NM_014791 | MELK | 0.53 | MELK | maternal embryonic leucine zipper kinase |
| NM_005733 | KIF20A | 0.53 | KIF20A | kinesin family member 20A |
| NM_018154 | ASF1B | 0.53 | ASF1B | ASF1 anti-silencing function 1 homolog B (S. cerevisiae) |
| NM_005542 | INSIG1 | 0.53 | INSIG1 | insulin induced gene 1 |
| NM_003600 | STK6 | 0.52 | STK6 | serine/threonine kinase 6 |
| NM_004701 | CCNB2 | 0.52 | CCNB2 | cyclin B2 |
| NM_004526 | MCM2 | 0.52 | MCM2 | MCM2 minichromosome maintenance |
| | | | | deficient 2, mitotin (S. cerevisiae) |
| U96131 | TRIP13 | 0.51 | TRIP13 | thyroid hormone receptor interactor 13 |
| NM_005192 | CDKN3 | 0.51 | CDKN3 | cyclin-dependent kinase inhibitor 3 (CDK2- |
| | | | | associated dual specificity phosphatase) |
| NM_001071 | TYMS | 0.51 | TYMS | thymidylate synthetase |
| Contig34952 | SHCBP1 | 0.51 | SHCBP1 | likely ortholog of mouse Shc SH2-domain |
| | | | | binding protein 1 |
| Contig46796_RC | C20orf172 | 0.51 | C20orf172 | chromosome 20 open reading frame 172 |
| Contig33814_RC | | 0.51 | ASPM | |
| NM_003626 | PPFIA1 | 0.51 | PPFIA1 | protein tyrosine phosphatase, receptor type, |
| | | | | f polypeptide (PTPRF), interacting protein |
| | | | | (liprin), alpha 1 |
| NW_016049 | LOC51016 | 0.51 | C14orf122 | |
| NM_003981 | PRC1 | 0.5 | PRC1 | |
| NM_006027 | EXO1 | 0.5 | EXO1 | exonuclease 1 |
| NM_004217 | STK12 | 0.5 | AURKB | aurora kinase B |
| NM_004162 | RAB5A | 0.5 | RAB5A | RAB5A, member RAS oncogene family |
| AL049685 | RAP2C | 0.5 | RAP2C | RAP2C, member of RAS oncogene family |
| AF052162 | FLJ12443 | 0.5 | FLJ12443 | hypothetical protein FLJ12443 |
| AL049949 | | −0.5 | FLJ90798 | |
| Contig31646_RC | COL14A1 | −0.5 | COL14A1 | collagen, type XIV, alpha 1 (undulin) |
| NM_003970 | MYOM2 | −0.5 | MYOM2 | myomesin (M-protein) 2, 165 kDa |
| Contig59870_RC | IRX1 | −0.5 | IRX1 | iroquois homeobox protein 1 |
| NM_013981 | NRG2 | −0.5 | NRG2 | neuregulin 2 |
| NM_000927 | ABCB1 | −0.5 | ABCB1 | ATP-binding cassette, sub-family B |
| | | | | (MDR/TAP), member 1 |
| AK001560 | NECL1 | −0.51 | NECL1 | nectin-like protein 1 |
| Contig52419_RC | JAM2 | −0.51 | JAM2 | junctional adhesion molecule 2 |
| Contig27623_RC | −0.51 | | | |
| NM_000114 | EDN3 | −0.51 | EDN3 | endothelin 3 |
| NM_002996 | CX3CL1 | −0.51 | CX3CL1 | chemokine (C—X3—C motif) ligand 1 |
| NM_013261 | PPARGC1 | −0.51 | PPARGC1 | |
| Contig46756_RC | SYN2 | −0.51 | SYN2 | synapsin II |
| NM_002405 | MFNG | −0.52 | MFNG | manic fringe homolog (Drosophila) |
| Contig43289_RC | | −0.52 | LOC170371 | |
| Contig45821_RC | ADCY4 | −0.52 | ADCY4 | adenylate cyclase 4 |
| NM_000820 | GAS6 | −0.52 | GAS6 | growth arrest-specific 6 |
| AF131817 | | −0.52 | CBFA2T1 | |
| Contig47308_RC | −0.52 | | | |
| Contig54993_RC | CLDN11 | −0.52 | CLDN11 | claudin 11 (oligodendrocyte transmembrane |
| | | | | protein) |
| NM_002001 | FCER1A | −0.52 | FCER1A | Fc fragment of IgE, high affinity I, receptor |
| | | | | for; alpha polypeptide |
| NM_000331 | SAA1 | −0.52 | SAA1 | serum amyloid A1 |
| NM_004787 | SLIT2 | −0.53 | SLIT2 | slit homolog 2 (Drosophila) |
| Contig43759_RC | | −0.53 | GRASP | |
| AL137698 | DKFZp434C1915 | −0.53 | PGM5 | phosphoglucomutase 5 |
| Contig42036_RC | BACH2 | −0.53 | BACH2 | BTB and CNC homology 1, basic leucine |
| | | | | zipper transcription factor 2 |
| NM_018286 | FLJ10970 | −0.54 | FLJ10970 | hypothetical protein FLJ10970 |
| Contig37198_RC | HSPA12B | −0.54 | HSPA12B | heat shock 70 kD protein 12B |
| NM_016602 | GPR2 | −0.54 | GPR2 | G protein-coupled receptor 2 |
| NM_004349 | CBFA2T1 | −0.54 | CBFA2T1 | core-binding factor, runt domain, alpha |
| | | | | subunit 2; translocated to, 1; cyclin D-related |
| AL110280 | | −0.54 | PAPLN | |
| NM_003012 | SFRP1 | −0.54 | SFRP1 | secreted frizzled-related protein 1 |
| NM_000109 | DMD | −0.54 | DMD | dystrophin (muscular dystrophy, Duchenne |
| | | | | and Becker types) |
| D00174 | SERPINF2 | −0.54 | SERPINF2 | serine (or cysteine) proteinase inhibitor, |
| | | | | clade F (alpha-2 antiplasmin, pigment |
| | | | | epithelium derived factor), member 2 |
| Contig42103_RC | | −0.55 | C20orf17 | chromosome 20 open reading frame 17 |
| Contig49869_RC | | −0.55 | | Homo sapiens cDNA FLJ31668 fis, clone |
| | | | | NT2RI2004916. |
| NM_001463 | FRZB | −0.55 | FRZB | frizzled-related protein |
| NM_004887 | CXCL14 | −0.55 | CXCL14 | chemokine (C—X—C motif) ligand 14 |
| AL080169 | DKFZP434C171 | −0.56 | DKFZP434C171 | DKFZP434C171 protein |
| NM_000587 | C7 | −0.56 | C7 | complement component 7 |
| NM_004615 | TM4SF2 | −0.56 | TM4SF2 | transmembrane 4 superfamily member 2 |
| NM_016250 | NDRG2 | −0.56 | NDRG2 | NDRG family member 2 |
| NM_003862 | FGF18 | −0.56 | FGF18 | fibroblast growth factor 18 |
| NM_012464 | TLL1 | −0.56 | TLL1 | tolloid-like 1 |
| Contig37540 | | −0.57 | | |
| Contig49279_RC | | −0.57 | FLJ25461 | hypothetical protein FLJ25461 |
| Contig26022_RC | MGC13057 | −0.58 | MGC13057 | hypothetical protein MGC13057 |
| NM_000076 | CDKN1C | −0.59 | CDKN1C | cyclin-dependent kinase inhibitor 1C (p57, |
| | | | | Kip2) |
| NM_004944 | DNASE1L3 | −0.6 | DNASE1L3 | deoxyribonuclease I-like 3 |
| NM_013999 | MEOX1 | −0.6 | MEOX1 | mesenchyme homeo box 1 |
| NM_005269 | GLI | −0.6 | GLI | glioma-associated oncogene homolog (zinc |
| | | | | finger protein) |
| NM_002101 | GYPC | −0.61 | GYPC | glycophorin C (Gerbich blood group) |
| NM_004867 | ITM2A | −0.61 | ITM2A | integral membrane protein 2A |
| NM_003956 | CH25H | −0.61 | CH25H | |
| NM_016815 | GYPC | −0.62 | GYPC | glycophorin C (Gerbich blood group) |
| NM_004527 | MEOX1 | −0.63 | MEOX1 | mesenchyme homeo box 1 |
| NM_003152 | STAT5A | −0.65 | STAT5A | signal transducer and activator of |
| | | | | transcription 5A |
| NM_002036 | FY | −0.67 | FY | Duffy blood group |

### 5.1.3 Identification of Markers

The present invention provides sets of markers for the identification of conditions or indications associated with breast cancer. Generally, the marker sets were identified by determining which of ˜25,000 human markers had expression patterns that correlate with the conditions or indications.

The methods for identification of sets of markers make use of measured cellular constituent profiles, e.g., expression profiles of a plurality of genes (e.g., measurements of abundance levels of the corresponding gene products), in tumor samples from a plurality of patients whose prognosis outcomes are known. The prognosis outcomes can be the prognosis at a predetermined time after initial diagnosis. The predetermined time can be any appropriate time period, e.g., 2, 3, 4, or 5 years. Prognosis markers can be obtained by identifying genes whose expression levels correlate with prognosis outcome, e.g., genes whose expression levels in good prognosis patients group are significantly different from those in poor prognosis patients. In preferred embodiments, the tumor samples from the plurality of patients are separated into a good prognosis group and a poor prognosis group for the predetermined time period. Genes whose expression levels exhibit differences between the good and poor prognosis groups to at least a predetermined level are selected as the genes whose expression levels correlate with patient prognosis. This section describes embodiments which employ genes and gene-derived nucleic acids as markers. However, it will be understood by a person skilled in the art that proteins or other cellular constituents may also be used as markers.

In a preferred embodiment, the expression profile is a differential expression profile. Each measurement in the profile is a differential expression level of a marker in a breast tumor sample versus that in a reference sample (also termed a standard or control sample). In one embodiment, the reference sample comprises polynucleotide molecules, derived from one or more samples from a plurality of normal individuals. For example, the normal individuals may be persons not having breast cancer. The standard or control may also comprise polynucleotide molecules, derived from one or more samples derived from individuals having a different form or stage of breast cancer; a different disease or different condition, or individuals exposed or subjected to a different condition, than the individual from which the sample of interest was obtained. The reference or control may be a sample, or set of samples, taken from the individual at an earlier time, for example, to assess the progression of a condition, or the response to a course of therapy.

In a preferred embodiment, the standard or control is a pool of target polynucleotide molecules derived from a plurality of different individuals. However, where protein levels, or the levels of any other relevant biomolecule, are to be compared, the pool may be a pool of proteins or the relevant biomolecule. In a preferred embodiment in the context of breast cancer, the pool comprises samples taken from a number of individuals having sporadic-type tumors.

In another preferred embodiment, the pool comprises an artificially-generated population of nucleic acids designed to approximate the level of nucleic acid derived from each marker found in a pool of marker-derived nucleic acids derived from tumor samples. In another embodiment, the pool, also called a “mathematical sample pool,” is represented by a set of expression values, rather than a set of physical polynucleotides; the level of expression of relevant markers in a sample from an individual with a condition, such as a disease, is compared to values representing control levels of expression for the same markers in the mathematical sample pool. Such a control may be a set of values stored on a computer. Such artificial or mathematical controls may be constructed for any condition of interest.

In another embodiment, the reference sample is derived from a normal breast cell line or a breast cancer cell line. Of course, where, for example, expressed proteins are used as markers, the proteins are obtained from the individual's sample, and the standard or control could be a pool of proteins from a number of normal individuals, or from a number of individuals having a particular state of a condition, such as a pool of samples from individuals having a particular prognosis of breast cancer.

In one embodiment, the method for identifying marker sets is as follows. After extraction and labeling of target polynucleotides, the expression of all markers (genes) in a sample X is compared to the expression of all markers in a standard or control. In one embodiment, the standard or control comprises target polynucleotide molecules derived from a sample from a normal individual (i.e., an individual not having breast cancer). In a preferred embodiment, the standard or control is a pool of target polynucleotide molecules. The pool may be derived from collected samples from a number of normal individuals. In a preferred embodiment, the pool comprises samples taken from a number of individuals having sporadic-type tumors. In another preferred embodiment, the pool comprises an artificially-generated population of nucleic acids designed to approximate the level of nucleic acid derived from each marker found in a pool of marker-derived nucleic acids derived from tumor samples. In yet another embodiment, the pool is derived from normal or breast cancer cell lines or cell line samples.

The comparison may be accomplished by any means known in the art. For example, expression levels of various markers may be assessed by separation of target polynucleotide molecules (e.g., RNA or cDNA) derived from the markers in agarose or polyacrylamide gels, followed by hybridization with marker-specific oligonucleotide probes. Alternatively, the comparison may be accomplished by the labeling of target polynucleotide molecules followed by separation on a sequencing gel. Polynucleotide samples are placed on the gel such that patient and control or standard polynucleotides are in adjacent lanes. Comparison of expression levels is accomplished visually or by means of densitometer. In a preferred embodiment, the expression of all markers is assessed simultaneously by hybridization to a microarray. In each approach, markers meeting certain criteria are identified as associated with breast cancer.

In one embodiment, genes are first screening genes based on significant variation in expression as compared to a standard or control sample in a set of breast cancer tumor samples. Genes may be screened, for example, by determining whether they show significant variation as compared to a standard or control sample in at least some samples among the set of samples. Genes that do not show significant variation in at least some samples in the set of samples are presumed not to be informative, and are discarded from further consideration. Genes showing significant variation in at least some samples in the sample set are retained as candidate informative genes. The degree of variation in expression of a gene may be estimated by determining a difference or ratio of the expression of the gene in a sample and a control. The difference or ratio of expression may be further transformed, e.g., by a linear or log transformation. Selection of candidate markers may be made based upon either significant up- or down-regulation of the gene in at least some samples in the set or based on the statistical significance (e.g., the p-value) of the variation in expression of the gene. Preferably, both selection criteria are used. Thus, in one embodiment of the present invention, genes showing both a more than two-fold change (increase or decrease) in expression as compared to a standard in at least three samples, and a p-value of variation in expression of the gene in the set of tumor samples as compared to the standard sample is no more than 0.01 (i.e., is statistically significant) are selected as candidate genes associated with prognosis of breast cancer.

Expression profiles comprising a plurality of different genes in a plurality of n breast cancer tumor samples can be used to identify markers that correlate with, and therefore are useful for discriminating, different clinical categories. In a specific embodiment using n tumor samples, markers are identified by calculation of correlation coefficients between the clinical category or clinical parameter(s) and the linear, logarithmic or any transform of the expression ratio across all samples for each individual gene. Specifically, the correlation coefficient may be calculated as:

ρ=(\vec{c}·\vec{r})/(∥\vec{c}∥·∥\vec{r}∥) Equation (1)

where \vec{c} represents the clinical parameters in the n tumor samples or categories and \vec{r} represents the measured expression levels of a gene in the n tumor samples, e.g., each element in \vec{r} can be the linear, logarithmic or any transform of the ratio of expression of the gene between a tumor sample and a control. Genes for which the coefficient of correlation exceeds a cutoff or threshold value are identified as breast cancer-related markers specific for a particular clinical type. Such a cutoff or threshold value may correspond to a certain significance of discriminating genes obtained by Monte Carlo simulations. The threshold depends upon the number of samples used, and can be calculated as 3×1/\sqrt{n−3}, where 1/\sqrt{n−3} is the distribution width and n=the number of samples. In a specific embodiment, markers are chosen if the correlation coefficient is greater than about 0.3 or less than about −0.3.

Next, the significance of the set of marker genes can be evaluated. The significance may be calculated by any appropriate statistical method. In a specific example, a Monte-Carlo technique is used to randomize the association between the expression profiles of the plurality of patients and the clinical categories to generate a set of randomized data. The same marker selection procedure as used to select the marker set is applied to the randomized data to obtain a control marker set. A plurality of such runs can be performed to generate a probability distribution of the number of genes in control marker sets. In a preferred embodiment, 10,000 such runs are performed. From the probability distribution, the probability of finding a marker set consisting of a given number of markers when no correlation between the expression levels and phenotype is expected (i.e., based randomized data) can be determined. The significance of the marker set obtained from the real data can be evaluated based on the number of markers in the marker set by comparing to the probability of obtaining a control marker set consisting of the same number of markers using the randomized data. In one embodiment, if the probability of obtaining a control marker set consisting of the same number of markers using the randomized data is below a given probability threshold, the marker set is said to be significant.

Once a marker set is identified, the markers may be rank-ordered in order of significance of discrimination. One means of rank ordering is by the amplitude of correlation between the change in gene expression of the marker and the specific condition being discriminated. Another, preferred, means is to use a statistical metric. In a specific embodiment, the metric is a Fisher-like statistic:

$$t = ( 〈 {x}_{1} 〉 - 〈 {x}_{2} 〉 ) / \sqrt{[ \begin{matrix}{σ}_{1}^{2} ( {n}_{1} - 1 ) + \\ {σ}_{2}^{2} ( {n}_{2} - 1 )\end{matrix} ] / ( {n}_{1} + {n}_{2} - 1 ) / ( 1 / {n}_{1} + 1 / {n}_{2} )} \qquad Equation ( 2 )$$

In this equation, |x₁> is the error-weighted average of the log ratio of transcript expression measurements within a first clinical group (e.g., good prognosis), <x₂> is the error-weighted average of log ratio within a second, related clinical group (e.g., poor prognosis), σ₁ is the variance of the log ratio within the first clinical group (e.g., good prognosis), n₁ is the number of samples for which valid measurements of log ratios are available, σ₂ is the variance of log ratio within the second clinical group (e.g., poor prognosis), and n₂ is the number of samples for which valid measurements of log ratios are available. The t-value represents the variance-compensated difference between two means. The rank-ordered marker set may be used to optimize the number of markers in the set used for discrimination.

The rank-ordered marker set may be used to optimize the number of markers in the set used for discrimination. This is accomplished generally in a “leave one out” method as follows. In a first run, a subset, for example 5, of the markers from the top of the ranked list is used to generate a template, where out of X samples, X−1 are used to generate the template, and the status of the remaining sample is predicted. This process is repeated for every sample until every one of the X samples is predicted once. In a second run, additional markers, for example 5, are added, so that a template is now generated from 10 markers, and the outcome of the remaining sample is predicted. This process is repeated until the entire set of markers is used to generate the template. For each of the runs, type 1 error (false negative) and type 2 errors (false positive) are counted; the optimal number of markers is that number where the type 1 error rate, or type 2 error rate, or preferably the total of type 1 and type 2 error rate is lowest.

For prognostic markers, validation of the marker set may be accomplished by an additional statistic, a survival model. This statistic generates the probability of tumor distant metastasis as a function of time since initial diagnosis. A number of models may be used, including Weibull, normal, log-normal, log logistic, log-exponential, or log-Rayleigh (Chapter 12 “Life Testing”, S-PLUS 2000 GUIDE TO STATISTICS, Vol. 2, p. 368 (2000)). For the “normal” model, the probability of distant metastasis P at time t is calculated as

P=α×exp(−t²/τ²) Equation (3)

where α is fixed and equal to 1, and τ is a parameter to be fitted and measures the “expected lifetime”.

It will be apparent to those skilled in the art that the above methods, in particular the statistical methods, described above, are not limited to the identification of markers associated with breast cancer, but may be used to identify set of marker genes associated with any phenotype. The phenotype can be the presence or absence of a disease such as cancer, or the presence or absence of any identifying clinical condition associated with that cancer. the phenotype may also be the response, or lack thereof, to a particular treatment regimen, for example, a course of one or more anticancer drugs. In the disease context, the phenotype may be a prognosis such as a survival time, probability of distant metastasis of a disease condition, or likelihood of a particular response to a therapeutic or prophylactic regimen. The phenotype need not be cancer, or a disease; the phenotype may be a nominal characteristic associated with a healthy individual.

In another embodiment, the invention provides an “iterative” method for the identification of sets of genes associated with a particular phenotype. An important aspect of this method is that samples, within a set of samples used to construct a classifier for the phenotype, that are incorrectly predicted using classifier templates constructed using all samples in the set, are discarded, and samples the phenotype of which is accurately predicted are retained. The retained samples are then used to construct a second classifier, which is more likely to contain a set of genes that reflects the dominant underlying molecular mechanism for the particular phenotype.

In one embodiment, therefore, the invention provides a method for determining a set of marker genes whose expression is associated with a particular phenotype, comprising the steps of: (a) selecting phenotype having two or more phenotype categories; (b) identifying a first plurality of genes, wherein the expression of said genes in a first plurality of samples is correlated or anticorrelated with one of the phenotype categories; (c) predicting the phenotype category of each sample in said plurality of samples based on the expression level of each of said plurality of genes across all other samples in said plurality of samples; (d) selecting those samples for which the phonotype category is correctly predicted, to form a second plurality of samples; and (e) identifying a second plurality of genes, wherein the expression of said genes in said second plurality of samples is correlated or anticorrelated with one of the phenotype categories; wherein said second plurality of genes is a set of marker genes whose expression is associated with a particular phenotype. In a specific embodiment, the phenotype is breast cancer. In a more specific embodiment, said phenotype categories are good prognosis and poor prognosis. In an even more specific embodiment, said good prognosis means no reoccurrence or metastasis within five years of initial diagnosis of breast cancer, and poor prognosis means reoccurrence or metastasis within five years of initial diagnosis of breast cancer. In another specific embodiment, said phenotype categories are response and non-response to a particular anticancer drug, or to a particular combination of anticancer drugs.

This iterative method, of course, may be applied to any disease or condition for which two or more phenotype categories exist. The method may be applied to the original generation of sets of markers informative for a particular phenotype and phenotype category(ies), and may be used to improve existing sets of markers that were selected by less robust means.

It should be noted that each of the markers identified as being phenotype and/or phenotype category-informative may be considered likely targets for therapeutics for that phenotype. For example, markers identified as breast cancer prognosis-informative represent genes, and/or their encoded proteins, that are targets for therapeutics against breast cancer.

### 5.1.4 Sample Collection

In the present invention, target polynucleotide molecules are extracted from a sample taken from an individual having breast cancer. The sample may be collected in any clinically acceptable manner, but must be collected such that marker-derived polynucleotides (i.e., RNA) are preserved. mRNA or nucleic acids derived therefrom (i.e., cDNA or amplified DNA) are preferably labeled distinguishably from standard or control polynucleotide molecules, and both are simultaneously or independently hybridized to a microarray comprising some or all of the markers or marker sets or subsets described above. Alternatively, mRNA or nucleic acids derived therefrom may be labeled with the same label as the standard or control polynucleotide molecules, wherein the intensity of hybridization of each at a particular probe is compared. A sample may comprise any clinically relevant tissue sample, such as a tumor biopsy or fine needle aspirate, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines. The sample may also be paraffin-embedded tissue sections (see, e.g., U.S. Patent Application Publication No. 2005/0048542A1, which is incorporated by reference herein in its entirety). The expression profiles of paraffin-embedded tissue samples are preferably obtained using quantitative reverse transcriptase polymerase chain reaction qRT-PCR (see Section 5.4.2.7., infra).

Methods for preparing total and poly(A)+ RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifigation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNAse inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLEcULAR BIOLOGY, vol. 2, Current Protocols Publishing, New York (1994). Once bound, poly(A)+ mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences. More preferably, the mRNA molecules of the RNA sample comprise mRNA molecules corresponding to each of the marker genes. In another specific embodiment, the RNA sample is a mammalian RNA sample.

In a specific embodiment, total RNA or mRNA from cells are used in the methods of the invention. The source of the RNA can be cells of a plant or animal, human, mammal, primate, non-human animal, dog, cat, mouse, rat, bird, yeast, eukaryote, prokaryote, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

Probes to the homologs of the marker sequences disclosed herein can be employed preferably wherein non-human nucleic acid is being assayed.

### 5.2 Methods of Using Breast Cancer Marker Sets

The present invention provides methods of using the marker sets to analyze a sample from an individual so as to determine the metastatic potential of an individual's tumor at a molecular level, i.e., to determine a prognosis for the individual from which the sample is obtained. The individual need not actually be having breast cancer. Essentially, the expression of specific marker genes in the individual, or a sample taken therefrom, is analyzed, e.g., compared to a standard or control, to determine if the pattern of expression indicates a good or a poor prognosis. For example, assuming two breast cancer-related conditions, X and Y, one can compare the levels of expression of breast cancer prognostic markers for condition X in an individual to the respective levels of the marker-derived polynucleotides in a control, wherein the levels of expression in the control represent the levels of expression of the markers exhibited by samples having condition X. In this instance, if the expression of the markers in the individual's sample is substantially (i.e., statistically) similar to that of the control, then the individual is said to have condition X, whereas if the expression of the markers in the individual's sample is substantially (i.e., statistically) different from that of the control, then the individual does not have condition X. Where, as here, the choice is bimodal (i.e., a sample is either X or Y), if the individual does not have condition X, the individual can additionally be said to have condition Y. For example, conditions X and Y can be a good prognosis and a poor prognosis, respectively, as defined by the particular disease or condition, such as breast cancer, and the particular clinical status of the individual. Of course, the comparison to a control representing condition Y can also be performed. In this instance, if the expression of the markers in the individual's sample is substantially (i.e., statistically) similar to that of the control, then the individual is said to have condition Y. Preferably both are performed simultaneously, such that each control acts as both a positive and a negative control. The distinguishing result may thus either be a demonstrable difference from the expression levels (i.e., the amount of marker-derived RNA, or polynucleotides derived therefrom) represented by the control, or no significant difference.

Thus, in one embodiment, the method of determining a particular tumor-related status of an individual comprises the steps of (1) hybridizing labeled target polynucleotides from the individual to a microarray containing one of the above marker sets; (2) hybridizing standard or control polynucleotides molecules to the microarray, wherein the standard or control molecules are differentially labeled from the target molecules; and (3) determining the difference in transcript levels, or lack thereof, between the target and standard or control, wherein the difference, or lack thereof, determines the individual's tumor-related status. In a more specific embodiment, the standard or control molecules comprise marker-derived polynucleotides from a pool of samples from normal individuals, or a pool of tumor samples from individuals having sporadic-type tumors. In a preferred embodiment, the standard or control is an artificially-generated pool of marker-derived polynucleotides, which pool is designed to mimic the level of marker expression exhibited by clinical samples of normal or breast cancer tumor tissue having a particular clinical indication (i.e., good prognosis or poor prognosis; no reoccurrence or metastasis within five years of initial diagnosis or reoccurrence or metastasis within five years of initial diagnosis; etc.). In another specific embodiment, the control molecules comprise a pool derived from normal or breast cancer cell lines.

The present invention provides sets of markers useful for distinguishing “good prognosis” from “poor prognosis” tumor types. In a preferred embodiment, “good prognosis” means no reoccurrence or metastasis, in the individual from which the sample was taken, within five years of initial diagnosis, and “poor prognosis” means reoccurrence or metastasis within five years of initial diagnosis. Thus, in one embodiment of the above method, the level of polynucleotides (i.e., mRNA or polynucleotides derived therefrom) in a sample from an individual, expressed from the different markers provided in any of Tables 1-8 are compared to the level of expression of the same markers from a control, wherein the control comprises marker-related polynucleotides derived from samples obtained from individuals with no 5-year reoccurrence or metastasis, samples take from individuals having reoccurrence or metastasis within five years, or both. Preferably, the comparison is to both, and preferably the comparison is to polynucleotide pools from a number of “good prognosis” and “poor prognosis” samples, respectively. Where, for example, the individual's marker expression most closely resembles or correlates with the “good prognosis” control, and does not resemble or correlate with the “poor prognosis” control, the individual is classified as having a good prognosis. Where the pool is not pure “good prognosis” or “poor prognosis,” for example, a sporadic pool may be used. A set of experiments should be performed in which nucleic acids from individuals with known prognosis status are hybridized against the pool, in order to define the expression templates for the “good prognosis” and “poor prognosis” group. Nucleic acids from each individual with unknown prognosis status are hybridized against the same pool and the expression profile is compared to the template(s) to determine the individual's prognosis.

The control or standard may be presented in a number of different formats. For example, the control, or template, to which the expression of marker genes in a breast cancer tumor sample is compared may be the average absolute level of expression of each of the genes in a pool of marker-derived nucleic acids pooled from breast cancer tumor samples obtained from a plurality of breast cancer patients. In this case, the difference between the absolute level of expression of these genes in the control and in a sample from a breast cancer patient provides the degree of similarity or dissimilarity of the level of expression in the patient sample and the control. The absolute level of expression may be measured by the intensity of the hybridization of the nucleic acids to an array. In other embodiments, the values for the expression levels of the markers in both the patient sample and control are transformed (see Section 5.4.3). For example, the expression level value for the patient, and the average expression level value for the pool, for each of the marker genes selected, may be transformed by taking the logarithm of the value. Moreover, the expression level values may be normalized by, for example, dividing by the median hybridization intensity of all of the samples that make up the pool. The control may be derived from hybridization data obtained simultaneously with the patient sample expression data, or may constitute a set of numerical values stores on a computer, or on computer-readable medium.

In one embodiment, the invention provides for method of determining whether an individual having breast cancer will likely experience a relapse within five years of initial diagnosis (i.e., whether an individual has a poor prognosis) comprising (1) comparing the level of expression of at least ten of the different markers listed in any of Tables 1-8 in a sample taken from the individual to the level of the same markers in a standard or control, where the standard or control levels represent those found in an individual with a poor prognosis; and (2) determining whether the level of the marker-related polynucleotides in the sample from the individual is significantly different than that of the control, wherein if no substantial difference is found, the patient has a poor prognosis, and if a substantial difference is found, the patient has a good prognosis. Persons of skill in the art will readily see that the markers associated with good prognosis can also be used as controls. In a more specific embodiment, both controls are run.

Poor prognosis of breast cancer may indicate that a tumor is relatively aggressive, while good prognosis may indicate that a tumor is relatively nonaggressive. Therefore, the invention provides for a method of determining a course of treatment of a breast cancer patient, comprising determining whether the level of expression of at least 10 of the different markers listed in any of Tables 1-8, or one or more subsets thereof, correlates with the level of these markers in a sample representing a good prognosis expression pattern or a poor prognosis pattern; and determining a course of treatment, wherein if the expression correlates with the poor prognosis pattern, the tumor is treated as an aggressive tumor.

For the embodiments of the methods described in this section, any of the marker sets described in Section 5.1.2. can be used. For example, the full set of markers may be used (i.e., the complete set of different markers shown in any of Tables 1-8). Alternatively, all markers disclosed herein may be used, i.e., all 387 prognosis-informative markers. In other embodiments, subsets of the markers may be used. In a preferred embodiment, the prognosis of an individual is determined using the markers listed in any of Tables 1-4 are used. In another preferred embodiment, the individual is identified as being ER+, and the prognosis of an individual is determined using the markers listed in any of Tables 5-8 are used. An individual may be identified as ER+ or ER− by an acceptable means (e.g., northern blot analysis, SDS-PAGE analysis, or microarray analysis). The level of expression of the ER gene alone may be determined, whereby, for example, if the level of expression is, or is nearly, zero, the individual is ER−, and higher levels of expression indicate that the individual is ER+. Alternatively, one may identify an sample as ER− or ER+ using gene expression levels, for example, those disclosed in International Application Publication No. WO 02/103320. In other embodiments, the prognosis of an individual may be determined using one or more subsets of at least 10, 15, 20, 25, 30, 40, 50, 75, 100, 125, 150, or 175 of the different markers present in any one or more of Tables 1-8 (SEQ ID NOS:1-387), up to the total number of markers 387.

In other preferred embodiments, the prognosis of an individual is determined using only those markers listed in Table 1, Table 2, Table 3, Table 4, Table 5, Table 6, Table 7 or Table 8. In other embodiments, the prognosis of an individual may be determined using one or more subsets of at least 10, 15, 20, 25, 30, 40, 50, 75, 100, 125, 150, or 175 of the different markers present in any of Tables 1-8, up to the total number of markers in a Table. In other embodiments, the prognosis of an individual may be determined using one or more subsets of no more than 10, 15, 20, 25, 30, 40, 50, 75, 100, 125, 150, or 175 of the different markers present in any of Tables 1-8, up to the total number of markers in a Table. In a preferred embodiment, where the individual is ER+, the different markers, or subsets of different markers, used are those listed in any of Tables 5-8.

The invention provides a method for determining a prognosis of an individual having breast cancer, comprising classifying said individual as having a good prognosis or a poor prognosis based on an expression profile comprising measurements of expression levels of a plurality of genes in a cell sample taken from the individual, said plurality of genes comprising 10 different genes corresponding to the markers listed in any one or more of Tables 1, 3, 5 and 7 (SEQ ID NOS:1-387), wherein a good prognosis predicts no reoccurrence or metastasis within a predetermined period after initial diagnosis, and wherein a poor prognosis predicts reoccurrence or metastasis within said predetermined period after initial diagnosis. In one embodiment, the patient's cellular constituent profile comprising measurements of a set of markers, e.g., expression levels of marker genes, is evaluated to determine whether the profile indicates good prognosis or poor prognosis. In a preferred embodiment, the patient's prognosis is evaluated by comparing the cellular constituent profile to a predetermined cellular constituent template profile corresponding to a certain prognosis, e.g., a good prognosis template comprising measurements of the plurality of cellular constituents which are representative of levels of the cellular constituents in a plurality of good prognosis patients or a poor prognosis template comprising measurements of the plurality of cellular constituents which are representative of levels of the cellular constituents in a plurality of poor prognosis patients. Herein a good prognosis patient is a patient who has no reoccurrence or metastasis within a period of time after initial diagnosis, e.g., a period of 1, 2, 3, 4, 5 or 10 years, and a poor prognosis patient is a patient who has reoccurrence or metastasis within a period of time after initial diagnosis, e.g., a period of 1, 2, 3, 4, 5 or 10 years. In a preferred embodiment, both periods are 5 years.

The degree of similarity of the patient's cellular constituent profile to a template representing good or poor prognosis can be used to indicate whether the patient has good or poor prognosis. In a preferred embodiment, a patient is classified as having a good prognosis profile if the patient's cellular constituent profile has a high similarity to a good prognosis template, e.g., a similarity to a good prognosis template above a predetermined threshold value; and/or has a low similarity to a poor prognosis template, e.g., a similarity to a poor prognosis template no higher than a predetermined threshold value. In another embodiment, a patient is classified as having a poor prognosis profile if the patient's cellular constituent profile has a low similarity to a good prognosis template and/or has a high similarity to a poor prognosis template.

The similarity between the marker expression profile of an individual and that of a control or template can be assessed in a number of ways. In the simplest case, the profiles can be compared visually in a printout of expression difference data. Alternatively, the similarity can be calculated mathematically.

In one embodiment, the similarity between two patients x and y, or patient x and a template y, expressed as a similarity value, can be calculated using the following equation:

$$S = 1 - [ \begin{matrix}{\sum }_{i = 1}^{{N}_{v}} \frac{( {x}_{i} - \overline{x} )}{{σ}_{{x}_{i}}} \frac{( {y}_{i} - \overline{y} )}{{σ}_{{y}_{i}}} / \\ \sqrt{{\sum }_{i = 1}^{{N}_{v}} {( \frac{{x}_{i} - \overline{x}}{{σ}_{{x}_{i}}} )}^{2} {\sum }_{i = 1}^{{N}_{v}} {( \frac{{y}_{i} - \overline{y}}{{σ}_{{y}_{i}}} )}^{2}}\end{matrix} ] \qquad Equation ( 4 )$$

In this equation, x and y are two patients with components of log ratio xᵢ and yᵢ, i=1, . . . , N. Associated with every value xᵢ is error σₓ_{ᵢ}. The smaller the value of σₓ_{ᵢ}, the more reliable the measurement xᵢ. The error-weighted arithmetic mean may be calculated using the following formula:

$$\overline{x} = {\sum }_{i = 1}^{{N}_{v}} \frac{{x}_{i}}{{σ}_{{x}_{i}}^{2}} / {\sum }_{i = 1}^{{N}_{v}} \frac{1}{{σ}_{{x}_{i}}^{2}} \qquad Equation ( 5 )$$

In a preferred embodiment, templates are developed for sample comparison. The template can be defined as the error-weighted log ratio average of the expression difference for the group of marker genes able to differentiate the particular breast cancer-related condition. For example, templates are defined for “good prognosis” samples and for “poor prognosis” samples. Next, a classifier parameter is calculated. This parameter may be calculated using either expression level differences between the sample and template, or by calculation of a correlation coefficient. In one embodiment, the similarity is represented by a correlation coefficient between the patient's profile and the template. In one embodiment, a correlation coefficient above a correlation threshold indicates high similarity, whereas a correlation coefficient below the threshold indicates low similarity. In preferred embodiments, the correlation threshold is set as 0.3, 0.4, 0.5 or 0.6. In another embodiment, similarity between a patient's profile and a template is represented by a distance between the patient's profile and the template. In one embodiment, a distance below a given value indicates high similarity, whereas a distance equal to or greater than the given value indicates low similarity.

Either one or both of the two classifier parameters (P₁ and P₂) can then be used to measure degrees of similarities between a patient's profile and the templates: P₁ measures the similarity between the patient's profile \vec{y} and the good prognosis template \vec{z}₁, and P₂ measures the similarity between \vec{y} and the poor prognosis template \vec{z}₂. Such a coefficient, Pᵢ, can be calculated using the following equation:

Pᵢ=(\vec{z}ᵢ·\vec{y})/(∥\vec{z}ᵢ∥·∥\vec{y}∥) Equation (6).

Thus, in one embodiment, \vec{y} is classified as a good prognosis profile if P₁ is greater than a selected correlation threshold or if P₂ is equal to or less than a selected correlation threshold. In another embodiment, \vec{y} is classified as a poor prognosis profile if P₁ is less than a selected correlation threshold or if P₂ is above a selected correlation threshold. In still another embodiment, \vec{y} is classified as a good prognosis profile if P₁ is greater than a first selected correlation threshold and \vec{y} is classified as a poor prognosis profile if P₂ is greater than a second selected correlation threshold.

Thus, in a more specific embodiment, the above method of determining a particular tumor-related status of an individual, i.e., prognosis, comprises the steps of (1) hybridizing labeled target polynucleotides from an individual to a microarray containing one of the above marker sets; (2) hybridizing standard or control polynucleotides molecules to the microarray, wherein the standard or control molecules are differentially labeled from the target molecules; and (3) determining the ratio (or difference) of transcript levels between two channels (individual and control), or simply the transcript levels of the individual; and (4) comparing the results from (3) to the predefined templates, wherein said determining is accomplished by means of the statistic of Equation 4 or Equation 6, and wherein the difference, or lack thereof, determines the individual's tumor-related status (for example, prognosis).

The invention further provides a method for classifying a breast cancer patient according to prognosis, comprising comparing the levels of expression of at least 10 of the different genes for which markers are listed in any of Tables 1-8 in a cell sample taken from said breast cancer patient to control levels of expression of said at least five genes; and classifying said breast cancer patient according to prognosis of his or her breast cancer based on the similarity between said levels of expression in said cell sample and said control levels. In a more specific embodiment, the second step of this method comprises determining whether said similarity exceeds one or more predetermined threshold values of similarity. In another more specific embodiment of this method, said control levels are the mean levels of expression of each of said at least ten genes in a pool of tumor samples obtained from a plurality of breast cancer patients having a good prognosis, e.g., who have no metastasis within five years of initial diagnosis. In another more specific embodiment of this method, said control levels comprise the expression levels of said genes in breast cancer patients who have had no metastasis within five years of initial diagnosis. In yet another more specific embodiment of this method, said control levels comprise, for each of said at least ten of the different genes for which markers are listed in any of Tables 1-8, mean log intensity values stored on a computer. In yet another more specific embodiment of this method, said control levels comprise, for each of said at least ten of the genes for which markers are listed in any of Tables 1-8, mean log intensity values stored on a computer. The set of mean log intensity values listed in this table may be used as a “good prognosis” template for any of the prognostic methods described herein. The above method may also compare the level of expression of at least 10, 20, 30, 40, 50, 75, 100 or more different genes for which markers listed in any of Tables 1-8, or each of the genes for which markers are listed in Table 1, Table 2, Table 3, Table 4, Table 5, Table 6, Table 7 or Table 8.

The present invention further provides a method of further classifying “good prognosis” patients into two groups: those having a “very good prognosis” and those having an “intermediate prognosis.” For each of the above classifications, the invention further provides recommended therapeutic regimens.

The present invention also provides for the classification of a breast cancer patient into one of three prognostic categories comprising (a) determining the similarity between the level of expression of at least ten of the different genes for which markers are listed in any of Tables 1-8 to control levels of expression to obtain a patient similarity value; (b) providing a first threshold similarity value that differentiates persons having a good prognosis from those having a poor prognosis, and providing determining a second threshold similarity value, where said second threshold similarity value indicates a higher degree of similarity of the expression of said genes to said control than said first similarity value; and (c) classifying the breast cancer patient into a first prognostic category if the patient similarity value exceeds the first and second threshold similarity values, a second prognostic category if the patient similarity value equals or exceeds the first but not the second threshold similarity value, and a third prognostic category if the patient similarity value is less than the first threshold similarity value. In a more specific embodiment, the levels of expression of each of said at least five genes is determined first. As above, the control comprises marker-related polynucleotides derived from breast cancer tumor samples taken from breast cancer patients clinically determined to have a good prognosis (“good prognosis” control), breast cancer patients clinically determined to have a poor prognosis “poor prognosis” control), or both. In a preferred embodiment, the control is a “good prognosis” control or template, i.e., a control or template comprising the mean levels of expression of said genes in breast cancer patients who have had no distant metastasis within five years of initial diagnosis. In another more specific embodiment, said control levels comprise a set of values, for example mean log intensity values, preferably normalized, stored on a computer. In another specific embodiment, said determining in step (a) may be accomplished by a method comprising determining the difference between the absolute expression level of each of said genes and the average expression level of the same genes in a pool of tumor samples obtained from a plurality of breast cancer patients who have had no relapse of breast cancer within five years of initial diagnosis. In another specific embodiment, said determining in step (a) may be accomplished by a method comprising determining the degree of similarity between the level of expression of each of said genes in a breast cancer tumor sample taken from a breast cancer patient and the level of expression of the same genes in a pool of tumor samples obtained from a plurality of breast cancer patients who have had no relapse of breast cancer within five years of initial diagnosis.

In a specific embodiment of the above method, said first threshold similarity value and said second threshold similarity values are selected by a method comprising (a) rank ordering in descending order said tumor samples that compose said pool of tumor samples by the degree of similarity between the level of expression of said genes in each of said tumor samples to the mean level of expression of the same genes of the remaining tumor samples that compose said pool to obtain a rank-ordered list, said degree of similarity being expressed as a similarity value; (b) determining an acceptable number of false negatives in said classifying, wherein said false negatives are breast cancer patients for whom the expression levels of said at least ten of the different genes for which markers are listed in any of Tables 1-8 in said cell sample predicts that said patient will have no distant metastasis within the first five years after initial diagnosis, but who has had a distant metastasis within the first five years after initial diagnosis; (c) determining a similarity value above which in said rank ordered list fewer than said acceptable number of tumor samples are false negatives; and (d) selecting said similarity value determined in step (c) as said first threshold similarity value; and (e) selecting a second similarity value, greater than said first similarity value, as said second threshold similarity value. In an even more specific embodiment of this method, said second threshold similarity value is selected in step (e) by a method comprising determining which of said tumor samples, taken from patients having a distant metastasis within five years of initial diagnosis, in said rank ordered list has the greatest similarity value, and selecting said greatest similarity value as said second threshold similarity value. In even more specific embodiments, said first and second threshold similarity values are correlation coefficients, and said first threshold similarity value is 0.4 and said second threshold similarity value is greater than 0.4. In another specific embodiment, said first similarity value is a similarity value above which at most 10% false negatives are predicted in a training set of tumors, and said second correlation coefficient is a coefficient above which at most 5% false negatives are predicted in said training set of tumors. In another specific embodiment, said first correlation coefficient is a coefficient above which 10% false negatives are predicted in a training set of tumors, and said second correlation coefficient is a coefficient above which no false negatives are predicted in said training set of tumors. In the above and other embodiments, “false negatives” are patients classified by the expression of the marker genes as having a good prognosis, or who are predicted by such expression to have a good prognosis, but who actually do develop distant metastasis within five years.

In a specific embodiment of the above methods, the first, second and third prognostic categories are characterized as “very good prognosis,” “intermediate prognosis,” and “poor prognosis,” respectively. Patients classified into the first prognostic category (“very good prognosis”) are likely not to have a distant metastasis within five years of initial diagnosis. Patients classified as having an “intermediate prognosis” are also unlikely to have a distant metastasis within five years of initial diagnosis, but may be recommended to undergo a different therapeutic regimen than patients having a “very good prognosis” marker gene expression profile (see below). Patients classified into the third prognostic category (“poor prognosis”) are likely to have a distant metastasis within five years of initial diagnosis.

In a more specific embodiment, the similarity value is the degree of difference between the absolute (i.e., untransformed) level of expression of each of the genes in a tumor sample taken from a breast cancer patient and the mean absolute level of expression of the same genes in a control. In another more specific embodiment, the similarity value is calculated using expression level data that is transformed. In another more specific embodiment, the similarity value is expressed as a similarity metric, such as a correlation coefficient, representing the similarity between the level of expression of the marker genes in the tumor sample and the mean level of expression of the same genes in a plurality of breast cancer tumor samples taken from breast cancer patients.

In another specific embodiment, said first and second similarity values are derived from control expression data obtained in the same hybridization experiment as that in which the patient expression level data is obtained. In another specific embodiment, said first and second similarity values are derived from an existing set of expression data. In a more specific embodiment, said first and second correlation coefficients are derived from a mathematical sample pool. For example, comparison of the expression of marker genes in new tumor samples may be compared to the pre-existing template determined for these genes for patients in a previous study; the template, or average expression levels of each of the marker genes can be used as a reference or control for any tumor sample. Preferably, the comparison is made to a template comprising the average expression level of at least ten of the different genes listed in any of Tables 1-8 for the 108 out of 153 patients (see Examples) clinically determined to have a good prognosis. The coefficient of correlation of the level of expression of these genes in the tumor sample to the “good prognosis” patient template is then determined to produce a tumor correlation coefficient. For this control patient set, two similarity values may be derived: a first correlation coefficient that minimizes Type 1 and Type 2 error, and a second correlation coefficient that is higher than the first correlation coefficient. The second correlation coefficient is that of the actual poor prognosis sample in the rank-ordered list of samples having the highest correlation to the “good prognosis” template. The value of the second correlation coefficient will depend upon the set of samples selected for generation of the template. New breast cancer patients whose coefficients of correlation of the expression of these marker genes with the “good prognosis” template equal or exceed the second correlation coefficient are classified as having a “very good prognosis”; those having a coefficient of correlation of between the first and second correlation coefficients are classified as having an “intermediate prognosis”; and those having a correlation coefficient lower than the first correlation coefficient are classified as having a “poor prognosis.”

Because the above methods may utilize arrays to which fluorescently-labeled marker-derived target nucleic acids are hybridized, the invention also provides a method of classifying a breast cancer patient according to prognosis, e.g., a breast cancer patient 55+ years of age or older, comprising the steps of (a) contacting first nucleic acids derived from a tumor sample taken from said breast cancer patient, and second nucleic acids derived from two or more tumor samples from breast cancer patients who have had no distant metastasis within five years of initial diagnosis, with an array under conditions such that hybridization can occur, detecting at each of a plurality of discrete loci on said array a first fluorescent emission signal from said first nucleic acids and a second fluorescent emission signal from said second nucleic acids that are bound to said array under said conditions, wherein said array comprises at least ten of the different genes for which markers are listed in any of Tables 1-4 and wherein at least 50% of the probes on said array are listed in Tables 1-8; (b) calculating the similarity between said first fluorescent emission signals and said second fluorescent emission signals across said at least ten genes; and (c) classifying said breast cancer patient according to prognosis of his or her breast cancer based on the similarity between said first fluorescent emission signals and said second fluorescent emission signals across said at least ten genes.

Once patients have been classified as having a “very good prognosis,” “intermediate prognosis” or “poor prognosis,” this information can be combined with the patient's clinical data to determine an appropriate treatment regimen. In one embodiment, the patient's lymph node metastasis status (i.e., whether the patient is pN+ or pN0) is determined. Patients who are pN0 and have a “very good prognosis” or “intermediate” expression profile may be treated without adjuvant chemotherapy. All other patients should be treated with adjuvant chemotherapy. In a more specific embodiment, the patient's estrogen receptor status is also identified (i.e., whether the patient is ER+ or ER−). Here, patients classified as having an “intermediate prognosis” or “poor prognosis” who are ER+ are assigned a therapeutic regimen that additionally comprises adjuvant hormonal therapy.

Thus, the invention provides for a method of assigning a therapeutic regimen to a breast cancer patient, e.g., a breast cancer patient 55+ years of age or older, comprising (a) classifying said patient as having a “poor prognosis,” “intermediate prognosis,” or “very good prognosis” on the basis of the levels of expression of at least ten of the different genes for which markers are listed in any of Tables 1-8; and (b) assigning said patient a therapeutic regimen, said therapeutic regimen comprising no adjuvant chemotherapy if the patient is lymph node negative and is classified as having a good prognosis or an intermediate prognosis, or comprising chemotherapy if said patient has any other combination of lymph node status and expression profile. In another embodiment, the invention provides a method for assigning a therapeutic regimen for a breast cancer patient, comprising determining the lymph node status for said patient; determining the level of expression of at least ten of the different genes listed in any of Tables 1-8 in a tumor sample from said patient, thereby generating an expression profile; classifying said patient as having a “poor prognosis”, “intermediate prognosis” or “very good prognosis” on the basis of said expression profile; and assigning the patient a therapeutic regimen, said therapeutic regimen comprising no adjuvant chemotherapy if the patient is lymph node negative and is classified as having a good prognosis or an intermediate prognosis, or a therapeutic regiment comprising chemotherapy if said patient has any other combination of lymph node status and expression profile. In a more specific embodiment of the above methods, the ER status of the patient is additionally determined, and if the breast cancer patient is ER(+) and has an intermediate or poor prognosis, the therapeutic regimen additionally comprises hormonal therapy. In another more specific embodiment is to determine the lymph node status and expression profiles, and to assign intermediate prognosis patients adjuvant hormonal therapy (whether or not ER status has been determined). In another specific embodiment, the breast cancer patient is premenopausal. In another specific embodiment, the breast cancer patient has stage I or stage II breast cancer.

The use of marker sets is not restricted to the prognosis of breast cancer-related conditions, and may be applied in a variety of phenotypes or conditions, clinical or experimental, in which gene expression plays a role. Where a set of markers has been identified that corresponds to two or more phenotypes, the marker set can be used to distinguish these phenotypes. For example, the phenotypes may be the diagnosis and/or prognosis of clinical states or phenotypes associated with other cancers, other disease conditions, or other physiological conditions, wherein the expression level data is derived from a set of genes correlated with the particular physiological or disease condition. Further, the expression of markers specific to other types of cancer may be used to differentiate patients or patient populations for those cancers for which different therapeutic regimens are indicated.

### 5.3 Improving Sensitivity to Expression Level Differences

In using the markers disclosed herein, and, indeed, using any sets of markers to differentiate an individual having one phenotype from another individual having a second phenotype, one can compare the absolute expression of each of the markers in a sample to a control; for example, the control can be the average level of expression of each of the markers, respectively, in a pool of individuals. To increase the sensitivity of the comparison, however, the expression level values are preferably transformed in a number of ways.

For example, the expression level of each of the markers can be normalized by the average expression level of all markers the expression level of which is determined, or by the average expression level of a set of control genes. Thus, in one embodiment, the markers are represented by probes on a microarray, and the expression level of each of the markers is normalized by the mean or median expression level across all of the genes represented on the microarray, including any non-marker genes. In a specific embodiment, the normalization is carried out by dividing the median or mean level of expression of all of the genes on the microarray. In another embodiment, the expression levels of the markers are normalized by the mean or median level of expression of a set of control markers. In a specific embodiment, the control markers comprise a set of housekeeping genes. In another specific embodiment, the normalization is accomplished by dividing by the median or mean expression level of the control genes.

The sensitivity of a marker-based assay will also be increased if the expression levels of individual markers are compared to the expression of the same markers in a pool of samples. Preferably, the comparison is to the mean or median expression level of each the marker genes in the pool of samples. Such a comparison may be accomplished, for example, by dividing by the mean or median expression level of the pool for each of the markers from the expression level each of the markers in the sample. This has the effect of accentuating the relative differences in expression between markers in the sample and markers in the pool as a whole, making comparisons more sensitive and more likely to produce meaningful results that the use of absolute expression levels alone. The expression level data may be transformed in any convenient way; preferably, the expression level data for all is log transformed before means or medians are taken.

In performing comparisons to a pool, two approaches may be used. First, the expression levels of the markers in the sample may be compared to the expression level of those markers in the pool, where nucleic acid derived from the sample and nucleic acid derived from the pool are hybridized during the course of a single experiment. Such an approach requires that new pool nucleic acid be generated for each comparison or limited numbers of comparisons, and is therefore limited by the amount of nucleic acid available. Alternatively, and preferably, the expression levels in a pool, whether normalized and/or transformed or not, are stored on a computer, or on computer-readable media, to be used in comparisons to the individual expression level data from the sample (i.e., single-channel data).

Thus, the current invention provides the following method of classifying a first cell or organism as having one of at least two different phenotypes, where the different phenotypes comprise a first phenotype and a second phenotype. The level of expression of each of a plurality of genes in a first sample from the first cell or organism is compared to the level of expression of each of said genes, respectively, in a pooled sample from a plurality of cells or organisms, the plurality of cells or organisms comprising different cells or organisms exhibiting said at least two different phenotypes, respectively, to produce a first compared value. The first compared value is then compared to a second compared value, wherein said second compared value is the product of a method comprising comparing the level of expression of each of said genes in a sample from a cell or organism characterized as having said first phenotype to the level of expression of each of said genes, respectively, in the pooled sample. The first compared value is then compared to a third compared value, wherein said third compared value is the product of a method comprising comparing the level of expression of each of the genes in a sample from a cell or organism characterized as having the second phenotype to the level of expression of each of the genes, respectively, in the pooled sample. Optionally, the first compared value can be compared to additional compared values, respectively, where each additional compared value is the product of a method comprising comparing the level of expression of each of said genes in a sample from a cell or organism characterized as having a phenotype different from said first and second phenotypes but included among the at least two different phenotypes, to the level of expression of each of said genes, respectively, in said pooled sample. Finally, a determination is made as to which of said second, third, and, if present, one or more additional compared values, said first compared value is most similar, wherein the first cell or organism is determined to have the phenotype of the cell or organism used to produce said compared value most similar to said first compared value.

In a specific embodiment of this method, the compared values are each ratios of the levels of expression of each of said genes. In another specific embodiment, each of the levels of expression of each of the genes in the pooled sample is normalized prior to any of the comparing steps. In a more specific embodiment, the normalization of the levels of expression is carried out by dividing by the median or mean level of the expression of each of the genes or dividing by the mean or median level of expression of one or more housekeeping genes in the pooled sample from said cell or organism. In another specific embodiment, the normalized levels of expression are subjected to a log transform, and the comparing steps comprise subtracting the log transform from the log of the levels of expression of each of the genes in the sample. In another specific embodiment, the two or more different phenotypes are different stages of a disease or disorder. In still another specific embodiment, the two or more different phenotypes are different prognoses of a disease or disorder. In yet another specific embodiment, the levels of expression of each of the genes, respectively, in the pooled sample or said levels of expression of each of said genes in a sample from the cell or organism characterized as having the first phenotype, second phenotype, or said phenotype different from said first and second phenotypes, respectively, are stored on a computer or on a computer-readable medium.

In a specific embodiment, the two phenotypes are good prognosis and poor prognosis. In a more specific embodiment, the two phenotypes are no metastasis within five years of initial diagnosis of breast cancer, and reoccurrence or metastasis within five years of initial diagnosis of breast cancer.

In another specific embodiment, the comparison is made between the expression of each of the genes in the sample and the expression of the same genes in a pool representing only one of two or more phenotypes. In the context of prognosis-correlated genes, for example, one can compare the expression levels of prognosis-related genes in a sample to the average level of the expression of the same genes in a “good prognosis” pool of samples (as opposed to a pool of samples that include samples from patients having poor prognoses and good prognoses). Thus, in this method, a sample is classified as having a good prognosis if the level of expression of prognosis-correlated genes exceeds a chosen coefficient of correlation to the average “good prognosis” expression profile (i.e., the level of expression of prognosis-correlated genes in a pool of samples from patients having a “good prognosis.” Patients whose expression levels correlate more poorly with the “good prognosis” expression profile (i.e., whose correlation coefficient fails to exceed the chosen coefficient) are classified as having a poor prognosis. The method can be applied to subdivisions of these prognostic classes. For example, in a specific embodiment, the phenotype is good prognosis and said determination comprises (1) determining the coefficient of correlation between the expression of said plurality of genes in the sample and of the same genes in said pooled sample; (2) selecting a first correlation coefficient value between 0.4 and +1 and a second correlation coefficient value between 0.4 and +1, wherein said second value is larger than said first value; and (3) classifying said sample as “very good prognosis” if said coefficient of correlation equals or is greater than said second correlation coefficient value, “intermediate prognosis” if said coefficient of correlation equals or exceeds said first correlation coefficient value, and is less than said second correlation coefficient value, or “poor prognosis” if said coefficient of correlation is less than said first correlation coefficient value.

Of course, single-channel data may also be used without specific comparison to a mathematical sample pool. For example, a sample may be classified as having a first or a second phenotype, wherein the first and second phenotypes are related, by calculating the similarity between the expression of at least 5 markers in the sample, where the markers are correlated with the first or second phenotype, to the expression of the same markers in a first phenotype template and a second phenotype template, by (a) labeling nucleic acids derived from a sample with a fluorophore to obtain a pool of fluorophore-labeled nucleic acids; (b) contacting said fluorophore-labeled nucleic acid with a microarray under conditions such that hybridization can occur, detecting at each of a plurality of discrete loci on the microarray a flourescent emission signal from said fluorophore-labeled nucleic acid that is bound to said microarray under said conditions; and (c) determining the similarity of marker gene expression in the individual sample to the first and second templates, wherein if said expression is more similar to the first template, the sample is classified as having the first phenotype, and if said expression is more similar to the second template, the sample is classified as having the second phenotype.

### 5.4 Determination of Marker Gene Expression Levels

### 5.4.1 Methods

The expression levels of the marker genes in a sample may be determined by any means known in the art. The expression level may be determined by isolating and determining the level (i.e., amount) of nucleic acid transcribed from each marker gene. Alternatively, or additionally, the level of specific proteins translated from mRNA transcribed from a marker gene may be determined.

The level of expression of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

These examples are not intended to be limiting; other methods of determining RNA abundance are known in the art.

The level of expression of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al, 1990, GEL ELECTROPHORESIS OF PROTEINS: A PRACTICAL APPROACH, IRL Press, New York; Shevchenko et al., Proc. Nat'l Acad. Sci. USA 93:1440-1445 (1996); Sagliocco et al., Yeast 12:1519-1533 (1996); Lander, Science 274:536-539 (1996). The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array and their binding is assayed with assays known in the art. Generally, the expression, and the level of expression, of proteins of diagnostic or prognostic interest can be detected through immunohistochemical staining of tissue slices or sections.

Finally, expression of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

### 5.4.2 Microarrays

In preferred embodiments, polynucleotide microarrays are used to measure expression so that the expression status of each of the markers above is assessed simultaneously. In a specific embodiment, the invention provides oligonucleotide or cDNA arrays comprising probes hybridizable to the genes corresponding to each of the marker sets described above (i.e., markers to distinguish patients 55 years and older with good prognosis versus patients with poor prognosis). In a more specific embodiment, the invention provides oligonucleotide arrays comprising probes having sequences identified by SEQ ID NOS: 388-774, corresponding respectively to markers identified by SEQ ID NOS: 1-387, or a subset or subsets of at least 10, 20, 30, 40, 50, 75, 100, 125, 150, 175 or 200 of these probes.

The microarrays provided by the present invention may comprise probes hybridizable to the genes corresponding to markers able to distinguish the status of one, two, or all three of the clinical conditions noted above. In particular, the invention provides polynucleotide arrays comprising probes to a subset or subsets of at least 10, 20, 30, 40, 50, 75, 100, 125, 150, 175 or 200 of the different markers for which genes are listed in any of Tables 1-8.

In specific embodiments, the invention provides polynucleotide arrays in which polynucleotide probes complementary and hybridizable to the breast cancer prognosis-related markers described herein are at least 50%, 60%, 70%, 80%, 85%, 90%, 95% or 98% of the probes on said array. In another specific embodiment, the microarray of the invention comprises probes to at least 10 genes selected from Table 1, Table 2, Table 3, Table 4, Table 5, Table 6, Table 7 or Table 8. In another specific embodiment, the microarray of the convention comprises probes complementary and hybridizable to 10%, 15%, 20%, 25%, 30%, 40%, 50%, 60%, 70%, 80%, or 90% of the genes listed in Table 1, Table 2, Table 3, Table 4, Table 5, Table 6, Table 7 or Table 8. Probes may be generated, of course, from the sequence of any of SEQ ID NOS: 1-387 for inclusion in a microarray of the invention. Preferably, a microarray of the invention comprises probes to all 200 genes listed in Tables 1 or 2; all 100 genes listed in Tables 3 or 4; all 200 genes listed in Tables 5 or 6; and/or all 100 genes listed in Tables 7 or 8. In another embodiment, the microarray of the invention comprises probes complementary and hybridizable to at least 10 of the genes listed in Tables 1-4, and probes complementary and hybridizable to at least 10 of the genes listed in Tables 5-8. The microarray may comprise probes complementary and hybridizable to 10%, 15%, 20%, 25%, 30%, 40%, 50%, 60%, 70%, 80%, or 90% of the different markers listed in any of Tables 1-8; that is, may comprise probes complementary and hybridizable to 10%, 15%, 20%, 25%, 30%, 40%, 50%, 60%, 70%, 80%, or 90% of the sequences of SEQ ID NOS:1-387.

In yet another specific embodiment, microarrays that are used in the methods disclosed herein optionally comprise markers additional to at least some of the different markers listed in Tables 1-8. For example, in a specific embodiment, the microarray is a screening or scanning array as described in Altschuler et al., International Publication WO 02/18646, published Mar. 7, 2002 and Scherer et al., International Publication WO 02/16650, published Feb. 28, 2002. The scanning and screening arrays comprise regularly-spaced, positionally-addressable probes derived from genomic nucleic acid sequence, both expressed and unexpressed. Such arrays may comprise probes corresponding to a subset of, or all of, the different markers listed in Tables 1-8, or a subset thereof as described above, and can be used to monitor marker expression in the same way as a microarray containing only markers listed in Tables 1-6.

In yet another specific embodiment, the microarray is a commercially-available cDNA microarray that comprises at least five of the different markers listed in Tables 1-8. Preferably, a commercially-available cDNA microarray comprises all of the markers listed in Tables 1-8. However, such a microarray may comprise 5, 10, 15, 25, 50, 100, 150, 200, 250 or more of the different markers in any of Tables 1-8, up to the total number of markers listed in Tables 1-8. In a specific embodiment of the microarrays used in the methods disclosed herein, the different markers that are all or a portion of Tables 1-8 are at least 50%, 60%, 70%, 80%, 90%, 95% or 98% of the probes on the microarray.

The microarray of the invention may additionally include sets of probes complementary and hybridizable to genes informative for related or unrelated conditions. For example, a microarray comprising probes complementary and hybridizable to a plurality of the different prognosis-informative genes listed in any or all of Tables 1-8 may additionally comprise probes complementary and hybridizable to genes informative for ER tumor status, genes that may be used to distinguish sporadic from BRCA-1 type tumors, or genes that are informative for any other clinical aspect of breast cancer, or any other related or unrelated condition.

General methods pertaining to the construction of microarrays comprising the marker sets and/or subsets above are described in the following sections.

### 5.4.2.1 Construction of Microarrays

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the markers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers is present on the array. In a preferred embodiment, the array comprises the 550 of the 2,460 RE-status markers, 70 of the BRCA1/sporadic markers, and all 231 of the prognosis markers.

### 5.4.2.2 Preparing Probes for Microarrays

As noted above, the “probe” to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROROCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001)).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

### 5.4.2.3 Attaching Probes to the Solid Surface

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in SYNTHETIC DNA ARRAYS IN GENETIC ENGINEERING, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

### 5.4.2.4 Target Polynucleotide Molecules

The polynucleotide molecules which may be analyzed by the present invention (the “target polynucleotide molecules”) may be from any clinically relevant source, but are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. No. 5,545,522, 5,891,636, or 5,716,785). Methods for preparing total and poly(A)⁺ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from cells of the various types of interest in this invention using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, which is preferred for S. cerevisiae, RNA is extracted from cells using phenol and chloroform, as described in Ausubel et al., eds., 1989, CURRENT PROTOCOLS IN MOLECULAR BIOLOGY, Vol III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived therefrom, is isolated from a sample taken from a person having breast cancer. Target polynucleotide molecules that are poorly expressed in particular cells may be enriched using normalization techniques (Bonaldo et al., 1996, Genome Res. 6:791-806).

As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the target polynucleotides. Alternatively, random primers may be used in conjunction with PCR methods or 17 promoter-based in vitro transcription methods in order to amplify the target polynucleotides.

In a preferred embodiment, the detectable label is a luminescent label. For example, fluorescent labels, bioluminescent labels, chemiluminescent labels, and calorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrine (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a standard. The standard can comprise target polynucleotide molecules from normal individuals (i.e., those not having breast cancer). In a highly preferred embodiment, the standard comprises target polynucleotide molecules pooled from samples from normal individuals or tumor samples from individuals having sporadic-type breast tumors. In another embodiment, the target polynucleotide molecules are derived from the same individual, but are taken at different time points, and thus indicate the efficacy of a treatment by a change in expression of the markers, or lack thereof, during and after the course of treatment (i.e., chemotherapy, radiation therapy or cryotherapy), wherein a change in the expression of the markers from a poor prognosis pattern to a good prognosis pattern indicates that the treatment is efficacious. In this embodiment, different timepoints are differentially labeled.

### 5.4.2.5 Hybridization to Microarrays

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A 93:10614 (1993)). Useful hybridization conditions are also provided in, e.g., Tijessen, 1993, HYBRIDIZATION WITH NUCLEIC ACID PROBES, Elsevier Science Publishers B.V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

### 5.4.2.6 Signal Detection and Data Analysis

When fluorescently labeled probes are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech 14:1681-1684 (1996), may be used to monitor mRNA abundance levels at a large number of sites simultaneously.

Signals are recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different breast cancer-related condition.

### 5.4.2.7. Expression Profiling Using RT-PCR

Quantitative reverse transcriptase PCR (qRT-PCR) can also be used to determine the expression level of a marker gene. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™. Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 770™ Sequence Detection System™. The system consists of a thermocycler, laser, charge-coupled device (CCD), camera and computer. The system includes software for running the instrument and for analyzing the data.

5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

### 5.5 Computer-Facilitated Analysis

The present invention further provides for kits comprising the marker sets above. In a preferred embodiment, the kit contains a microarray ready for hybridization to target polynucleotide molecules, plus software for the data analyses described above.

The analytic methods described in the previous sections can be implemented by use of the following computer systems and according to the following programs and methods. A computer system comprises internal components linked to external components. The internal components of a typical computer system include a processor element interconnected with a main memory. For example, the computer system can be an Intel 8086-, 80386-, 80486-, Pentium™, or Pentium™-based processor with preferably 32 MB or more of main memory. The computer system may also be a Macintosh or a Macintosh-based system, but may also be a minicomputer or mainframe.

The external components may include mass storage. This mass storage can be one or more hard disks (which are typically packaged together with the processor and memory). Such hard disks are preferably of 1 GB or greater storage capacity. Other external components include a user interface device, which can be a monitor, together with an inputting device, which can be a “mouse”, or other graphic input devices, and/or a keyboard. A printing device can also be attached to the computer.

Typically, a computer system is also linked to network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on the mass storage device. A software component comprises the operating system, which is responsible for managing computer system and its network interconnections. This operating system can be, for example, of the Microsoft Windows® family, such as Windows 3.1, Windows 95, Windows 98, Windows 2000, or Windows NT, or may be of the Macintosh OS family, or may be UNIX or an operating system specific to a minicomputer or mainframe. The software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, FORTRAN and JAVA. Most preferably, the methods of this invention are programmed in mathematical software packages that allow symbolic entry of equations and high-level specification of processing, including some or all of the algorithms to be used, thereby freeing a user of the need to procedurally program individual equations or algorithms. Such packages include Mathlab from Mathworks (Natick, Mass.), Mathematica® from Wolfram Research (Champaign, Ill.), or S-Plus® from Math Soft (Cambridge, Mass.). Specifically, the software component includes the analytic methods of the invention as programmed in a procedural language or symbolic package.

The software to be included with the kit comprises the data analysis methods of the invention as disclosed herein. In particular, the software may include mathematical routines for marker discovery, including the calculation of similarity values between clinical categories (e.g., ER status) and marker expression. The software may also include mathematical routines for calculating the similarity between sample marker expression and control marker expression, using array-generated fluorescence data, to determine the clinical classification of a sample.

Additionally, the software may also include mathematical routines for determining the prognostic outcome, and recommended therapeutic regimen, for a particular breast cancer patient. Such software would include instructions for the computer system's processor to receive data structures that include the level of expression of ten or more of the different marker genes listed in any of Tables 1-8 in a breast cancer tumor sample obtained from the breast cancer patient; the mean level of expression of the same genes in a control or template; and the breast cancer patient's clinical information, for example including lymph node and ER status. The software may additionally include mathematical routines for transforming the hybridization data and for calculating the similarity between the expression levels for the marker genes in the patient's breast cancer tumor sample and the control or template. In a specific embodiment, the software includes mathematical routines for calculating a similarity metric, such as a coefficient of correlation, representing the similarity between the expression levels for the marker genes in the patient's breast cancer tumor sample and the control or template, and expressing the similarity as that similarity metric.

The software may include decisional routines that integrate the patient's clinical and marker gene expression data, and recommend a course of therapy. In one embodiment, for example, the software causes the processor unit to receive expression data for the patient's tumor sample, calculate a metric of similarity of these expression values to the values for the same genes in a template or control, compare this similarity metric to a pre-selected similarity metric threshold or thresholds that differentiate prognostic groups, assign the patient to the prognostic group, and, on the basis of the prognostic group, assign a recommended therapeutic regimen. In a specific example, the software additionally causes the processor unit to receive data structures comprising clinical information about the breast cancer patient. In a more specific example, such clinical information includes the patient's age, stage of breast cancer, estrogen receptor status, and lymph node status.

Where the control is an expression template comprising expression values for marker genes within a group of breast cancer patients, the control can comprise either hybridization data obtained at the same time (i.e., in the same hybridization experiment) as the patient's individual hybridization data, or can be a set of hybridization or marker expression values stores on a computer, or on computer-readable media. If the latter is used, new patient hybridization data for the selected marker genes, obtained from initial or follow-up tumor samples, or suspected tumor samples, can be compared to the stored values for the same genes without the need for additional control hybridizations. However, the software may additionally comprise routines for updating the control data set, i.e., to add information from additional breast cancer patients or to remove existing members of the control data set, and, consequently, for recalculating the average expression level values that comprise the template. In another specific embodiment, said control comprises a set of single-channel mean hybridization intensity values for each of said at least ten of said genes, stored on a computer-readable medium.

Clinical data relating to a breast cancer patient, and used by the computer program products of the invention, can be contained in a database of clinical data in which information on each patient is maintained in a separate record, which record may contain any information relevant to the patient, the patient's medical history, treatment, prognosis, or participation in a clinical trial or study, including expression profile data generated as part of an initial diagnosis or for tracking the progress of the breast cancer during treatment.

Thus, one embodiment of the invention provides a computer program product for classifying a breast cancer patient according to prognosis, the computer program product for use in conjunction with a computer having a memory and a processor, the computer program product comprising a computer readable storage medium having a computer program mechanism encoded thereon, wherein said computer program product can be loaded into the one or more memory units of a computer and causes the one or more processor units of the computer to execute the steps of (a) receiving a first data structure comprising the level of expression of at least ten of the different genes for which markers are listed in any of Tables 1-8 in a cell sample taken from said breast cancer patient; (b) determining the similarity of the level of expression of said at least 10 genes to control levels of expression of said at least five genes to obtain a patient similarity value; (c) comparing said patient similarity value to selected first and second threshold values of similarity of said level of expression of said genes to said control levels of expression to obtain first and second similarity threshold values, respectively, wherein said second similarity threshold indicates greater similarity to said control levels of expression than does said first similarity threshold; and (d) classifying said breast cancer patient as having a first prognosis if said patient similarity value exceeds said first and said second threshold similarity values, a second prognosis if said patient similarity value exceeds said first threshold similarity value but does not exceed said second threshold similarity value, and a third prognosis if said patient similarity value does not exceed said first threshold similarity value or said second threshold similarity value. In a specific embodiment of said computer program product, said first threshold value of similarity and said second threshold value of similarity are values stored in said computer. In another more specific embodiment, said first prognosis is a “very good prognosis,” said second prognosis is an “intermediate prognosis,” and said third prognosis is a “poor prognosis,” and wherein said computer program mechanism may be loaded into the memory and further cause said one or more processor units of said computer to execute the step of assigning said breast cancer patient a therapeutic regimen comprising no adjuvant chemotherapy if the patient is lymph node negative and is classified as having a good prognosis or an intermediate prognosis, or comprising chemotherapy if said patient has any other combination of lymph node status and expression profile. In another specific embodiment, said computer program mechanism may be loaded into the memory and further cause said one or more processor units of the computer to execute the steps of receiving a data structure comprising clinical data specific to said breast cancer patient. In a more specific embodiment, said clinical data includes the lymph node and estrogen receptor (ER) status of said breast cancer patient. In more specific embodiment, said single-channel hybridization intensity values are log transformed. The computer implementation of the method, however, may use any desired transformation method. In another specific embodiment, the computer program product causes said processing unit to perform said comparing step (c) by calculating the difference between the level of expression of each of said genes in said cell sample taken from said breast cancer patient and the level of expression of the same genes in said control. In another specific embodiment, the computer program product causes said processing unit to perform said comparing step (c) by calculating the mean log level of expression of each of said genes in said control to obtain a control mean log expression level for each gene, calculating the log expression level for each of said genes in a breast cancer sample from said breast cancer patient to obtain a patient log expression level, and calculating the difference between the patient log expression level and the control mean log expression for each of said genes. In another specific embodiment, the computer program product causes said processing unit to perform said comparing step (c) by calculating similarity between the level of expression of each of said genes in said cell sample taken from said breast cancer patient and the level of expression of the same genes in said control, wherein said similarity is expressed as a similarity value. In more specific embodiment, said similarity value is a correlation coefficient. The similarity value may, however, be expressed as any art-known similarity metric.

In an exemplary implementation, to practice the methods of the present invention, a user first loads experimental data into the computer system. These data can be directly entered by the user from a monitor, keyboard, or from other computer systems linked by a network connection, or on removable storage media such as a CD-ROM, floppy disk (not illustrated), tape drive (not illustrated), ZIP® drive (not illustrated) or through the network. Next the user causes execution of expression profile analysis software which performs the methods of the present invention.

In another exemplary implementation, a user first loads experimental data and/or databases into the computer system. This data is loaded into the memory from the storage media or from a remote computer, preferably from a dynamic geneset database system, through the network. Next the user causes execution of software that performs the steps of the present invention.

Additionally, because the data obtained and analyzed in the software and computer system products of the invention are confidential, the software and/or computer system comprises access controls or access control routines, such as encryption, password-controlled access, and the like.

Alternative computer systems and software for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### 6. EXAMPLES

### Example 1

### Identification of Prognosis-Relevant Markers

A. Materials and Experimental Methods

153 tumor samples were collected from breast cancer patients, each of whom was at least 55 years of age. Of the 153 patients, 45 had metastasis and 108 had no metastasis. RNA samples from each patient were prepared, and each RNA sample was profiled using inkjet microarrays. Marker genes were then identified based on expression patterns, and classifiers were trained to use these marker genes to classify tumors into prognostic categories. These marker genes were then used to predict the prognostic outcome.

Amplification, Labeling, and Hybridization

Total RNA was extracted from flash-frozen biopsy tumor specimens from each of the 153 breast cancer patients by using RNeasy columns (Qiagen). 5 μg total RNA was used as input for cRNA synthesis. An oligo-dT primer containing a T7 RNA polymerase promoter sequence was used to prime first strand cDNA synthesis, and random primers (pdN6) were used to prime second strand cDNA synthesis by MMLV Reverse Transcriptase. This reaction yielded a double-stranded cDNA that contained the T7 RNA polymerase (M7RNAP) promoter. The double-stranded cDNA was then transcribed into cRNA by T7RNAP. cRNA was labeled with Cy3 or Cy5 dyes using a two-step process. First, allylamine-derivitized nucleotides were enzymatically incorporated into cRNA products. For cRNA labeling, a 3:1 mixture of 5-(3-Aminoallyl)uridine 5′-triphosphate (Sigma) and UTP was substituted for UTP in the in vitro transcription (IVT) reaction. Allylamine-derivitized cRNA products were then reacted with N-hydroxy succinimide esters of Cy3 or Cy5 (CyDye, Amersham Pharmacia Biotech). 5 μg Cy5-labeled cRNA from one breast cancer patient were mixed with the same amount of Cy3-labeled product from the pool of equal amount of cRNA from each individual sporadic patient. Hybridizations were done in duplicate with fluor reversals. Before hybridization, labeled cRNAs were fragmented to an average size of approximately 50-100 nucleotides by heating at 60° C. in the presence of 10 mM ZnCl₂. Fragmented cRNAs were added to hybridization buffer containing 1 M NaCl, 0.5% sodium sarcosine and 50 mM MES, pH 6.5, which stringency was regulated by the addition of formamide to a final concentration of 30%. Hybridizations were carried out in a final volume of 3 ml at 40° C. on a rotating platform in a hybridization oven (Robbins Scientific). After hybridization, slides were washed and scanned using a confocal laser scanner (Agilent Technologies). Fluorescence intensities on scanned images were quantified, normalized and corrected.

Pooling of Samples

The reference cRNA pool was formed by pooling equal amount of cRNAs from each individual patient.

25 k Human Microarray and Hybridization

Hybridizations were carried out in duplicate, the second time after fluorescent dye reversals. Before hybridization, labeled cRNAs were fragmented to an average size of approximately 50-100 nucleotides by heating at 60° C. in the presence of 10 mM ZnCl₂. Fragmented cRNAs were added to hybridization buffer containing 1 M NaCl, 0.5% sodium sarcosine and 50 mM MES, pH 6.5, and hybridization stringency was regulated by the addition of formamide to a final concentration of 30%. Hybridizations were carried out in a final volume of 3 ml at 40° C. on a rotating platform in a hybridization oven (Robbins Scientific). Hu25K microarrays represented the 24479 biological oligonucleotides plus 1281 control probes were used for this study. Sequences for microarrays were selected from RefSeq (a collection of non-redundant mRNA sequences, www.ncbi.nlm.nih.gov/LocusLink/refseq.html) and Phil Green EST contigs. Each mRNA or EST contig was represented on the Hu25K microarray by a single 60-mer oligonucleotide chosen by an oligo probe design program. After hybridization, slides were washed and scanned using a confocal laser scanner (Agilent Technologies). Fluorescence intensities on scanned images were quantified, normalized and corrected. Intensity ratios relative to the reference pool were calculated and the significance of the differential regulation was estimated by the error model developed for the transcript ratio measurements with two-color-labeled hybridization microarray system.

Analytic Methods and Primary Results

The methodological invention consists of four parts. The first part is the overview of the gene expression patterns from all 153 tumors from patient group of >55 year by two-dimensional unsupervised clustering to identify the dominant tumor types. The second part focuses on evaluating the 70-gene based classifier in the age group >55 years to test whether there is a prognostic profile that are universally valid across age groups (<55 year and >55 year) for breast cancer. In the third part, a group of marker genes was also identified that can be used to classify sporadic breast cancer patients with age >55 year into two different prognostic groups—poor prognosis group and good prognosis group. Finally, similar classifiers were identified for prognosis within patient groups with ER+.

B. Overall Expression Patterns of Breast Cancer Tumors in Patients with Age >55 year.

Of approximately 25,000 sequences represented on the array, a group of approximately 10,000 genes was selected that were significantly differentially expressed across the group of samples. A gene was deemed significant if (1) it was differentially expressed by more than two-fold, and (2) the absolute value of the p-value of significance for differential expression was less than 0.01 in at least 10 out of the 153 tumor samples. These selection criteria were guided by an error model developed for the null hypothesis of transcript ratio measurements with a two-color-labeled hybridization microarray system.

An unsupervised clustering algorithm was used to cluster tumors based on their similarities measured over the set of ˜10,000 significant genes. The similarity measure between two patients x and y was defined as

$$S = 1 - [ \begin{matrix}{\sum }_{i = 1}^{{N}_{v}} \frac{( {x}_{i} - \overline{x} )}{{σ}_{{x}_{i}}} \frac{( {y}_{i} - \overline{y} )}{{σ}_{{y}_{i}}} / \\ \sqrt{{\sum }_{i = 1}^{{N}_{v}} {( \frac{{x}_{i} - \overline{x}}{{σ}_{{x}_{i}}} )}^{2} {\sum }_{i = 1}^{{N}_{v}} {( \frac{{y}_{i} - \overline{y}}{{σ}_{{y}_{i}}} )}^{2}}\end{matrix} ] \qquad Equation ( 4 )$$

In Equation (1), x and y are two patients with components of log ratio xᵢ and yᵢ, i=1, . . . , N. Associated with every value xᵢ is error σₓ_{ᵢ}. The smaller the value σₓ_{ᵢ}, the more reliable the measurement xᵢ. The error-weighted arithmetic mean was calculated as;

$$\overline{x} = {\sum }_{i = 1}^{{N}_{v}} \frac{{x}_{i}}{{σ}_{{x}_{i}}^{2}} / {\sum }_{i = 1}^{{N}_{v}} \frac{1}{{σ}_{{x}_{i}}^{2}} \qquad Equation ( 5 )$$

The use of correlation as similarity metric emphasizes the importance of co-regulation in clustering rather than the amplitude of expression.

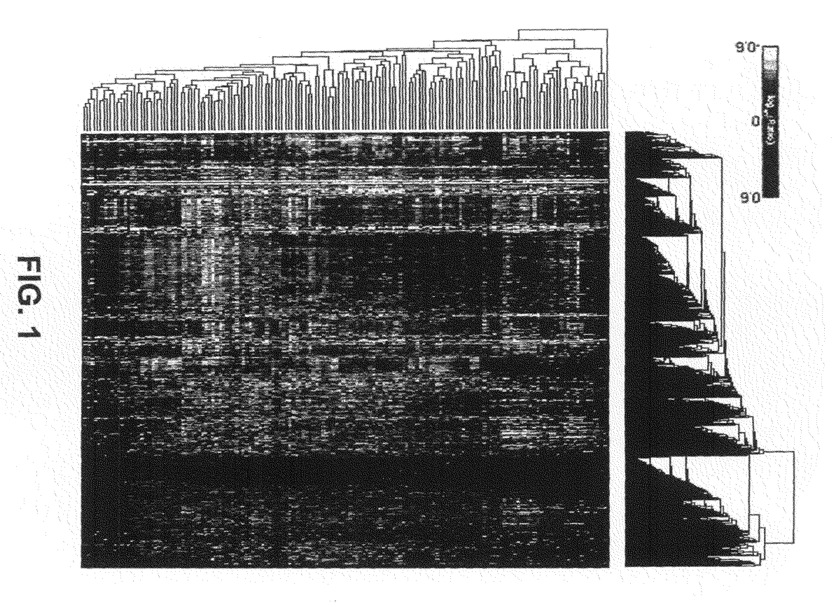

The set of ˜10,000 genes can also be clustered based on their similarities measured over the group of 153 tumor samples. The similarity measure between two genes is defined in the same way as in Equation (1) except that for each gene there are 153 components of log ratio measurements. The two-dimensional clustering results shown in FIG. 1 are genome-wide overview of data representation for the profiled 153 tumor samples. The overall pattern revealed by unsupervised clustering relates to the end-point of interest in this study, i.e., metastasis status. This indicates that the transcriptional profiles of RNA samples from breast tumors measured with microarray technology represent patient disease states of prognostic value, and therefore the use of supervised algorithms should allow identification of predictors and construction of classifiers to differentiate tumors by prognosis.

C. Testing Predictive Power of the 70-Gene Classifier for Breast Tumor Prognosis

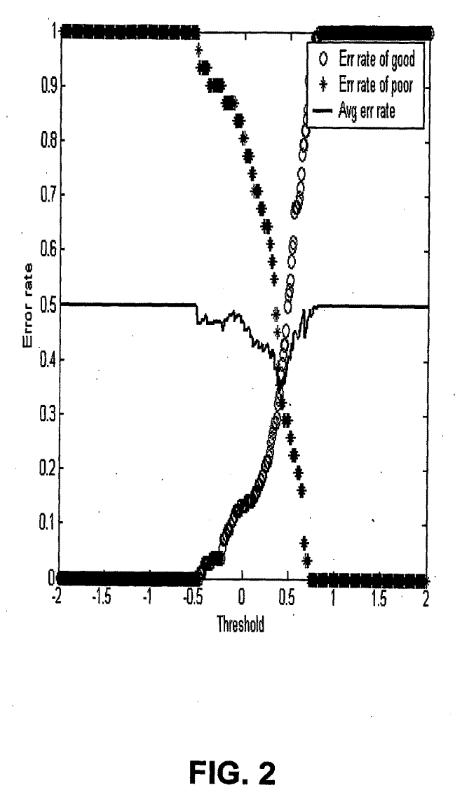

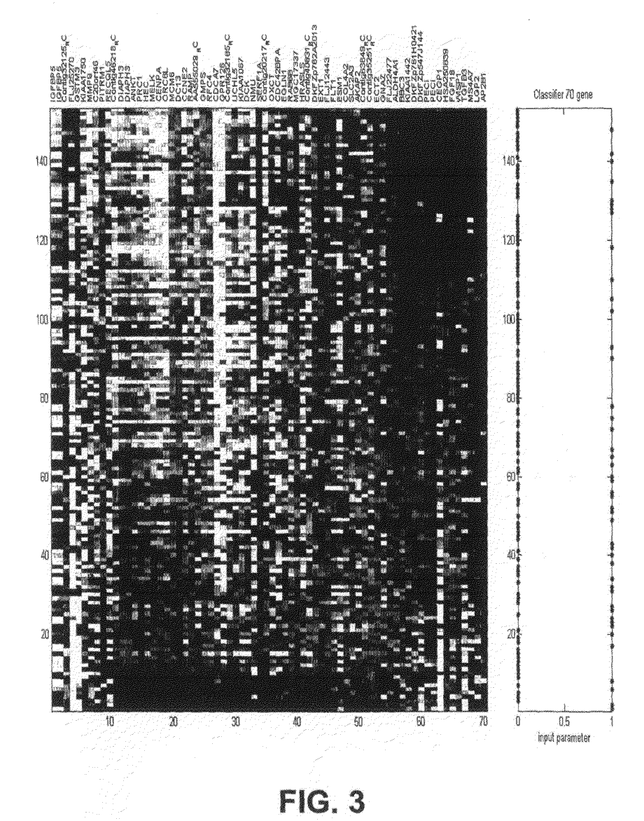

A 70-gene classifier previously described (van't Veer et al., Nature 415, 530-536 (2002)) was developed using samples from breast tumors from patients <55 years of age. The predictive power and performance of this 70-gene classifier was evaluated across two age groups. With the same procedure detailed in a previous study (van't Veer et al., Nature 415, 530-536 (2002)) and the same threshold used previously, the 70-gene classifier was used to divide all 153 tumor samples into two groups based on the expression of the 70 reporter genes, one with good prognosis and one with poor prognosis. The odds ratio was calculated for the predicted prognosis of all 153 tumor samples in comparison with actual clinical outcomes. The odds ratio of outcome prediction was found to be significant: 2.5 for the overall metastasis, and 5.2 for the 5-year metastasis. The 95% confidence interval is 1.2-5.1 for the overall metastasis and 2.0-13.0 for the 5-year metastasis. These numbers were obtained at a fixed threshold that were defined in our previous study for the age group of <55 years (see Van't Veer (2002)). FIG. 2 shows the total error rate (type 1+type 2 errors) as a function of threshold for overall metastasis of all 153 tumor samples. FIG. 3 shows the gene expression pattern of the 70-reporters for 153 profiled tumor samples. Visually, there are expression patterns in the group of 70 genes that are indicative of disease outcome among the 153 tumors. These results indicate that the classifier based on data from patients with age <55 years has predictive power in prognosis of breast tumors from patients with age >55 years.

D. Classification Method for Selecting Marker Genes as Prognostic Predictors for Breast Cancers of Patients with Age >55 Years

153 tumors from breast cancer patients with age >55 years were used to refine prognostic predictors from gene expression data for this age group. Of the 153 samples in this breast cancer group of age >55 years, 108 samples were from individuals that had no metastasis, among which 89 had a follow up time more than 5 years (collectively the 108 individuals are referred to as the “no-metastasis group”) and 45 samples exhibited metastasizes, among which 29 exhibited metastasis within 5 years of the initial diagnosis (collectively, the “metastasis group”). The goal was to identify a set of marker genes from this data set exhibiting certain expression patterns that allow differentiation of these two subgroups among “sporadic” patients in the age group of >55 years.

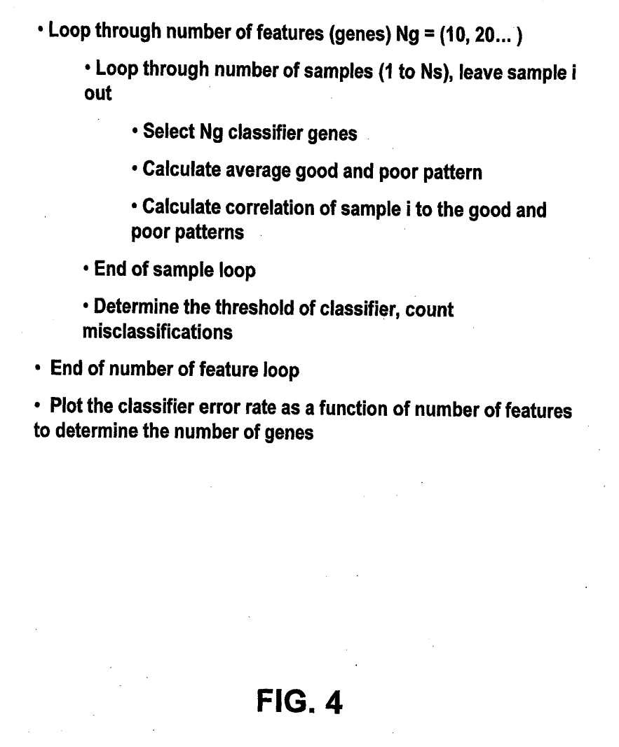

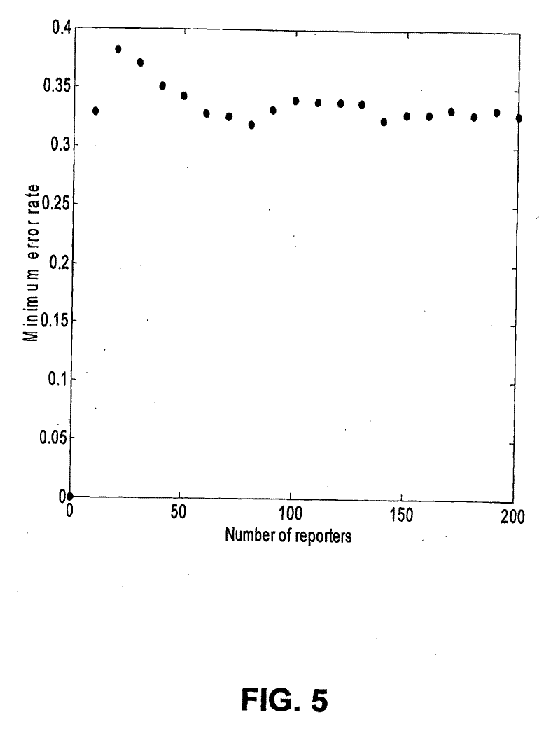

A “leave-one-out” cross-validation method was used to build and evaluate a classifier (See FIG. 4). In this method, one sample is reserved for cross validation each time the classifier is trained. The training of the classifier involves the following steps (1)-(3) for any reserved sample. Steps (1)-(3) are repeated N times for N samples so that each sample is reserved once. See van't Veer et al., Nature 415, 530-536 (2002).

Selection of Candidate Discriminating Genes

Non-informative genes in each group of patients were first filtered out. Only genes with | log 10(ratio)|>log 10(2) and P-value (for log(ratio)≠0)<0.01 in more than 3 experiments were selected for the classifier. This step removed all genes that showed no significant change across all samples. In the first step, a set of candidate discriminating genes was identified based on gene expression data of a subset of these 153 samples. The subset of samples used for feature selection were those from individuals having either a good outcome with a follow up time at least 5 years, or a poor outcome metastasized with in 5 years, and those that were not omitted. The correlation ρ between the prognostic category number (metastasis versus non-metastasis) \vec{c} and the logarithmic expression ratio \vec{r} across all tumor samples for each individual gene was calculated as:

ρ=(\vec{c}·\vec{r})/(∥\vec{c}∥·∥\vec{r}∥) Equation (1)

where both C and r in Equation (1) are mean subtracted. Although the majority of genes do not correlate with the prognostic categories, a small group of genes do correlate. Genes with larger correlation coefficients were used as reporters for the prognosis of interest—reoccurrence group and non-reoccurrence group.

Rank-Ordering of Candidate Discriminating Genes

In the second step, genes on the candidate list were rank-ordered based on the magnitude of correlation as calculated above.

Classification Based on Marker Genes

In the third step, a subset of N genes (as specified by the classifier) from the top of this rank-ordered list was used as discriminating genes. In particular, a template was defined for “good prognosis” group (called \vec{z}₁) by using the error-weighted log ratio average of the selected group of genes. Similarly, a template was defined for “poor prognosis” group (called \vec{z}₂) by using the error-weighted log ratio average of the selected group of genes. Two classifier parameters (P₁ and P₂) were defined based on either correlation or distance. P₁ measures the similarity between one sample \vec{y} and the “good prognosis” template \vec{z}₁ over this selected group of genes. P₂ measures the similarity between one sample \vec{y} and the “poor prognosis” template \vec{z}₂ over this selected group of genes. For correlation case, Pi is defined as:

Pᵢ=(\vec{z}ᵢ·\vec{y})/(∥\vec{z}ᵢ∥·∥\vec{y}∥) Equation (3).

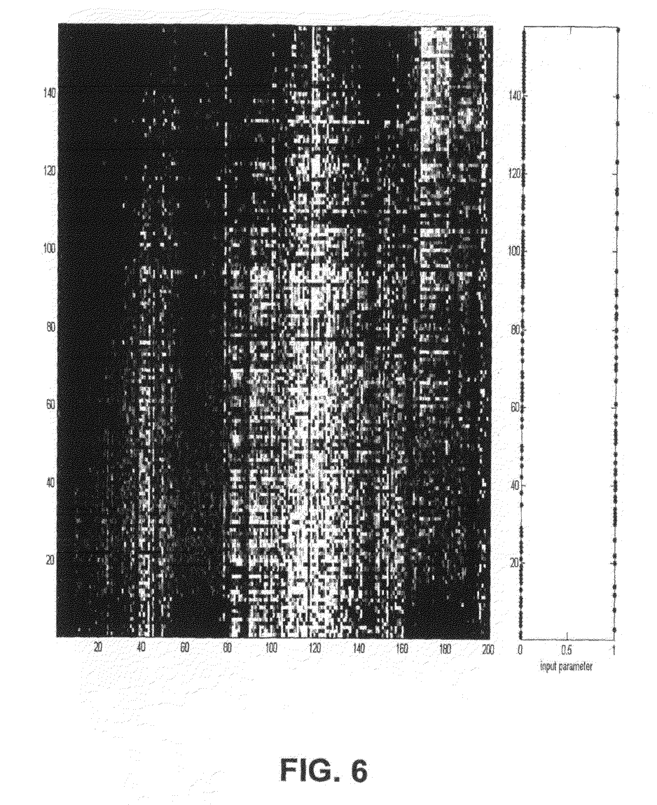

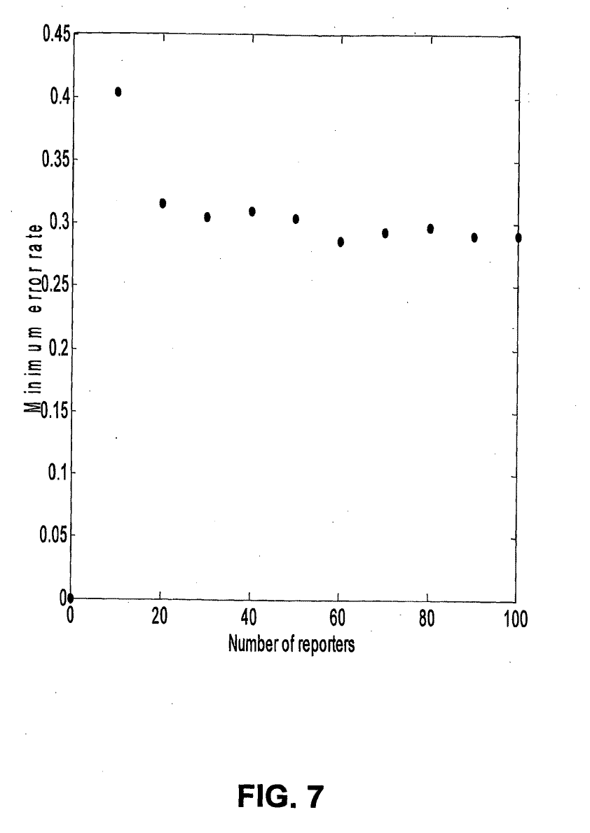

The performance of a classifier may vary with the number of features used in the classifier. To find the optimal number of features, the above process was repeated by varying the number of features (i.e., genes) starting from 10, and also in increments of 10, to several hundred genes. The error rate is quite stable for marker genes above 100 (see FIG. 7). A set of 200 genes was thus selected as the optimal set of marker genes to classify breast cancer tumors into “poor prognosis” group and “good prognosis” group (see Tables 1 and 2). The classification results made with this optimal set of 200 marker genes are shown in FIG. 6.

### Example 2

### Iterative Algorithm (“Homogenous Method”) to Build Classifier for Prognosis of Breast Tumors from Patients with Age >55 Years

Another optimal prognosis classifier was constructed using a different algorithm than that described above. The basic algorithm for classification used here is similar to the method previously used, except as noted below.

A. Feature Selection and Performance Evaluation:

Non-informative genes were first filtered in each group of patients. Specifically, only genes with | log 10(ratio)|>log 10(2) and P-value (for log(ratio)≠0)<0.01 in more than 3 experiments were selected. This step removed all genes that showed no significant change across all samples. The second step involved a double loop of a leave-one-out cross validation (LOOCV) procedure to select the training samples, classifier features and evaluate the performance. Even though all samples in each group were used to evaluate the classifier, only “training samples” were used to develop the classifier. In the leave-one-out process, if the left-out sample is one of the training samples, it is removed from the feature selection and classifier construction from that leave-one-out step. As above, the classifier features were selected according to correlation with outcome (i.e., good prognosis or poor prognosis). Because of the “iterative training sample selection,” the features selected from each step of the second loop of leave-one-out process were highly overlapping. The final “optimal” reporter genes were selected using all the “training samples” as the result of “re-substitution” because one classifier was needed for each group.

B. Identification of Homogeneous Patterns and Dominant Mechanism by “Iterative Training Sample Selection”:

In order to identify homogeneous patterns and reveal the dominant mechanisms, a classifier-building method called “iterative training sample selection” was used. In the first step of this method, only the samples of those patients who had metastasis shorter than 5 years or who were metastasis-free with more than 5 years of follow-up time were used as the training set. Based on these training samples, a complete LOOCV (including reselecting features) process was performed. During this step, the number of features was fixed at 50 genes. This number is chosen to provide a stable classifier by the algorithm. Training samples that were incorrectly predicted (samples from individuals with a poor prognosis correlating more to the average good prognosis profile than the average poor prognosis profile, or vice-versa) by this LOOCV process were removed from the training set in the second round of LOOCV. This is the opposite of the “boost” algorithm (see foe example, Achapire and Singer, Machine Learning 37(3):297-336 (1999)), which increases the weight of the misclassified samples in the training for the accuracy of the classifier. The current algorithm focuses on the most common prediction rule (that is, the mechanism of tumor development) within the data set by excluding “unpredictable,” or incorrectly-predicted, samples from the training set, ensuring robust feature selection. Biologically, for complicated diseases like cancer, there are likely some samples, in a set of samples from individuals with the disease, of tumors that do not develop by the most common mechanism. Including such samples tends to confuse feature selection for the predominant mechanism. Identification of the “unpredictable samples” in the first round of LOOCV, and exclusion of them from the training set of the second round, avoids a confounding factor in feature selection.

Using this method, a very homogeneous group of genes, many cell cycle-related, was selected. Due to the homogeneous pattern, the classifier accuracy was almost independent of the number of features. Even though the classifier accuracy was not the objective of the current algorithms, the iterative method resulted in an improved accuracy due to the robust feature set.

C. Error Rate and Odds Ratio, Threshold in the Final LOOCV:

Unless otherwise stated, the error rate is the average error rate from two populations: the number of poor outcome samples mis-classified as good outcome, divided by the total number of poor samples; and the number of good outcome samples mis-classified as poor outcome, divided by the total number of good samples. Two odds ratios are reported for a given threshold for differentiating good-outcome samples form poor-outcome samples: (1) the overall odds ratio; and (2) the 5 year odds ratio. The 5 year odds ratio was calculated from samples from individuals who were metastasis free for more than five years, or from individuals that had metastasis within 5 years).

The threshold was applied to cor1-cor2, where “cor1” stands for correlation to the “average good profile” in the training set, and “cor2” stands for the correlation to the “average poor profile” in the training set. The threshold in the final round of LOOCV was defined as follows. (1) For each of the N sample i left out for training, features were selected based on the training set. (2) Given a feature set, an incomplete LOOCV was performed using N−1 samples; only the “average poor profile” and “average good profile” was varied depending on whether the left out sample was in the training set or not. (3) A threshold is then determined based on the minimum error rate from the N−1 samples, and that threshold is assigned to sample i in step (1). This step was repeated for each sample i in the set of samples. (4) The mean threshold from all N samples was then calculated, and designated the final threshold. By this method, the threshold in the classifier did not necessarily correspond to the minimum error rate, hence avoiding overestimating the performance.

D. Correlation Calculation:

The correlation between expression log(ratio) and the endpoint data (final outcome) for each gene was calculated using the Pearson's correlation coefficient. The correlation between the profile and the “average good profile” and “average poor profile” for each tumor was the cosine product (no mean subtraction).

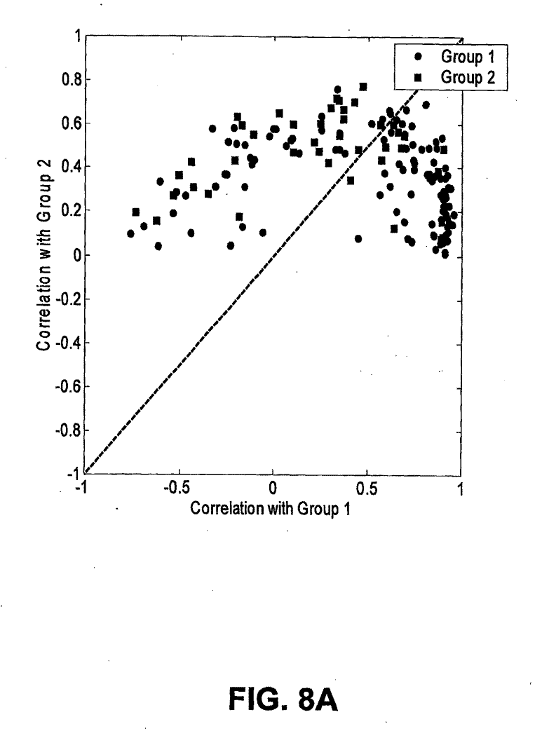

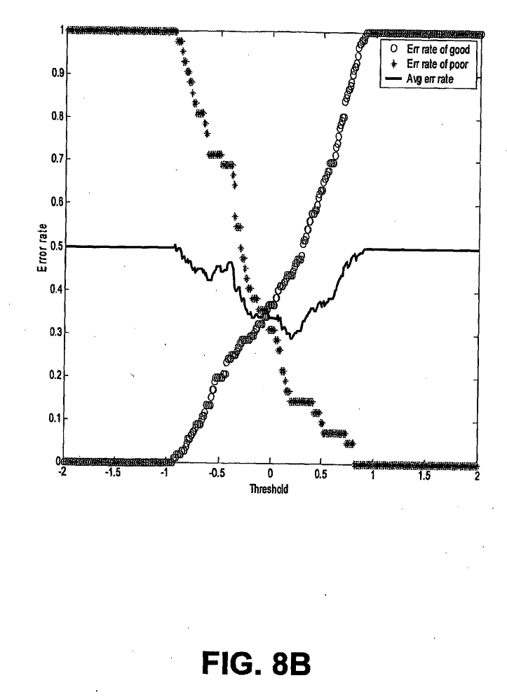

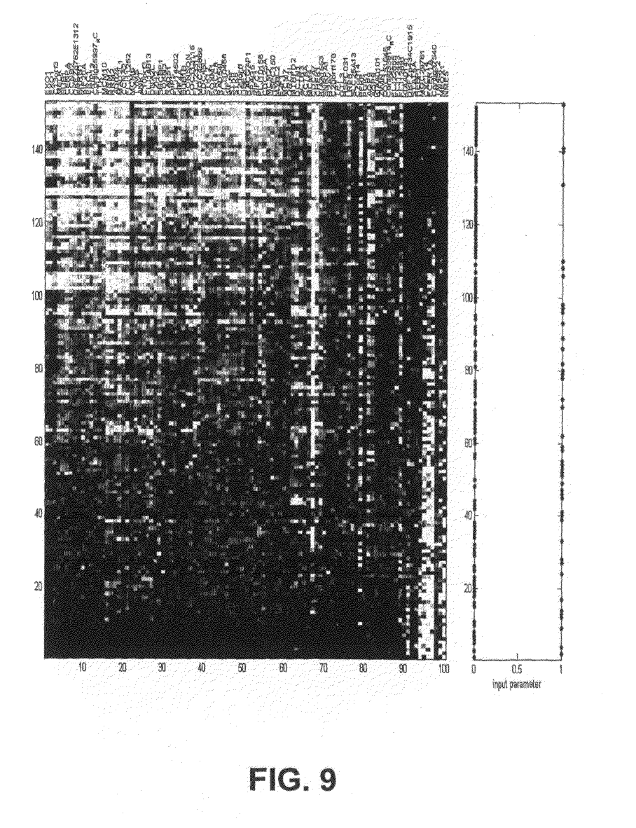

The total error rate as a function of the number of discriminating genes is shown in FIG. 7. Using the above method, an optimal set of 100 genes was identified that was used to build a classifier to predict the prognosis (see Tables 3 and 4). The scattering plot between correlation to “poor prognosis” profile and the correlation to “good prognosis” profile is shown in FIG. 8A. The type 1 error rate, the type 2 error rate, and average error rate are all shown in FIG. 8B as a function of threshold. The heatmap of gene expression for these 100 genes in all 153 samples is shown in FIG. 9.

### Example 3

### Comparison of Three Classifiers

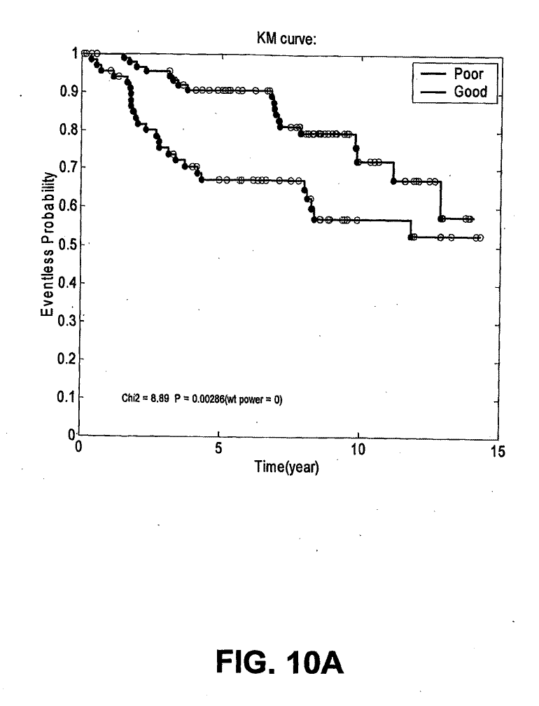

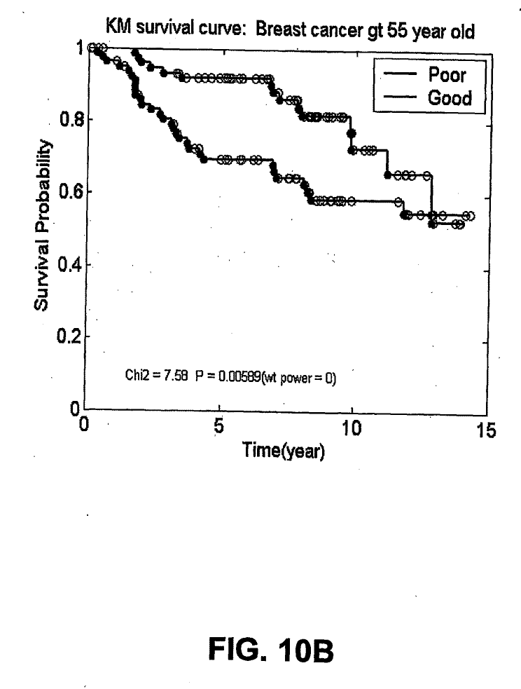

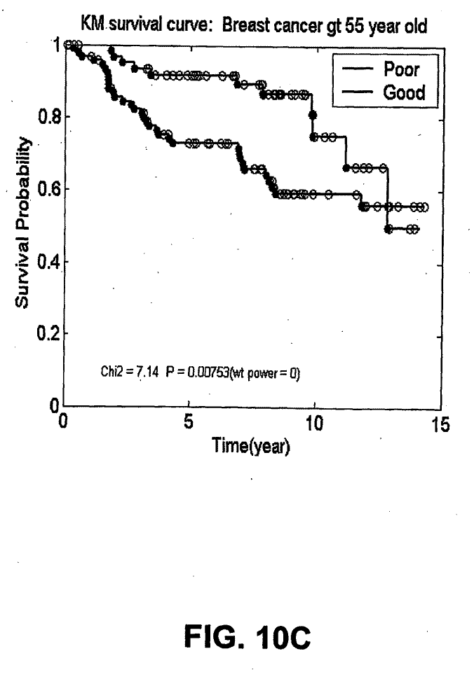

Table 9 summarizes the results of odds ratio, 95% confidence interval, total error rate, and p-value of log rank comparison test of two survival curves on Kalpan-Meier plots (FIG. 10) for predictions based on leave-one-out procedure from the previously constructed 70-gene based classifier, the 200-gene based classifier constructed by the same method, and the 100-gene based classifier constructed by the new (iterative) method.

TABLE 9
Comparison of three different classifier genesets in the prognosis
of samples from individuals age 55+.

| | | | | average |
|---|---|---|---|---|
| | Overall Odds | 5 year | average Error | Error Rate |
| | Ratio | Odds Ratio | Rate (overall) | (5 year) |
| 70 gene | 2.5 (1.2-5.1) | 5.2 (2.1-13.0) | 0.39 | 0.31 |
| Old method | 2.7 (1.3-5.6) | 5.4 (2.0-14.5) | 0.38 | 0.31 |
| New | 3.0 (1.4-6.5) | 4.5 (1.6-12.8) | 0.38 | 0.34 |
| method | | | | |

From the table and the K-M plots, it is evident that all three methods give similar results. The log rank test indicates that the separation into two prognosis groups has significance by all three classifiers (p<0.01). However, the significances are at similar levels (p=0.0029, 0.0059, and 0.0075 for the 70-gene model, the 200-gene model, and the 100-gene model, respectively).

### Example 4

### Marker Genes as Prognostic Predictors for Breast Cancers in Er+ Group

The estrogen receptor (ER) level (ER+ or ER−) affects the expression of thousands genes. It hence makes sense to develop a prognosis classifier separately for the ER+ patients and for the ER− patients. All 153 patient samples were divided into two groups, ER+ and ER−. Measurements from a microarray for ESR1 were used to determine the ER status. The threshold used was the same threshold established in the previous study (see Van't Veer (2002)). Samples with ESR1 log(ratio)>-0.65 were called ER+ samples. Of the 153 patients, 118 were ER+ and 35 were ER−. Because of the limited number of samples in the ER− group, only results derived from the ER+ group are discussed herein. Both the old and new method described above were used to build two separate classifiers for disease outcome prediction within ER+ group.

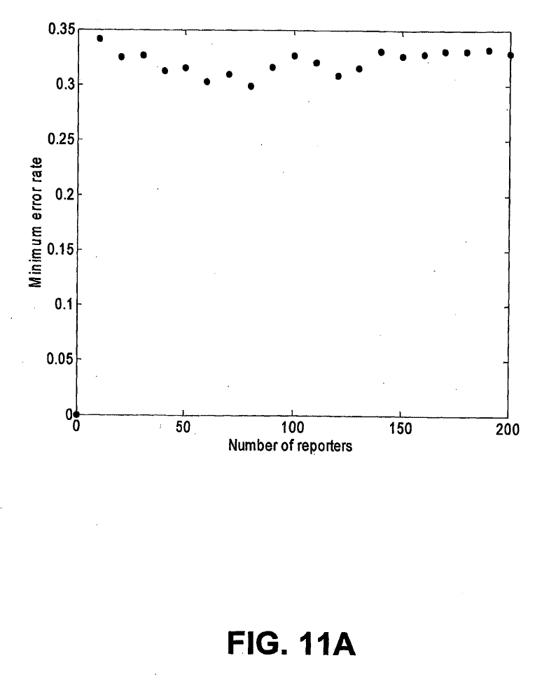

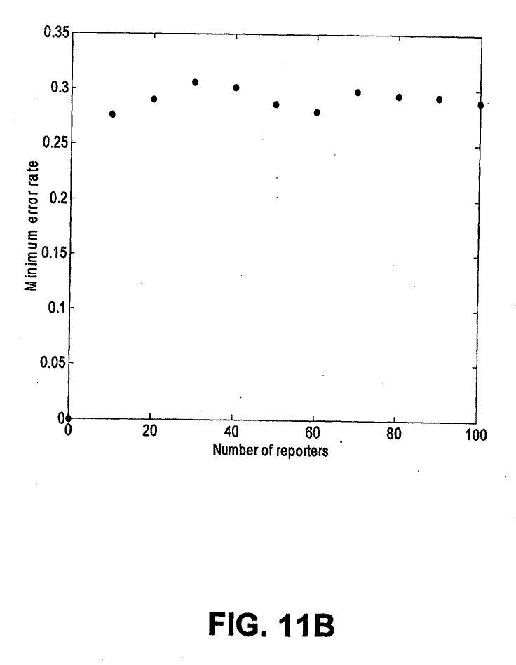

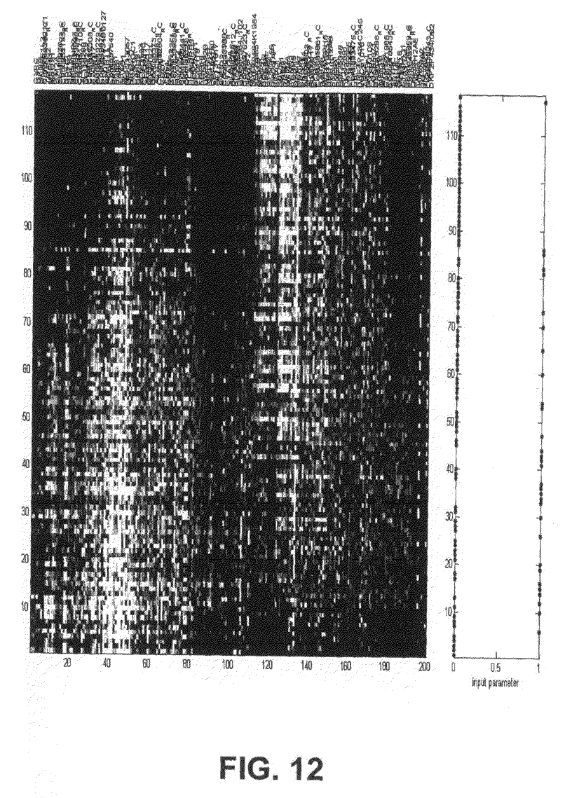

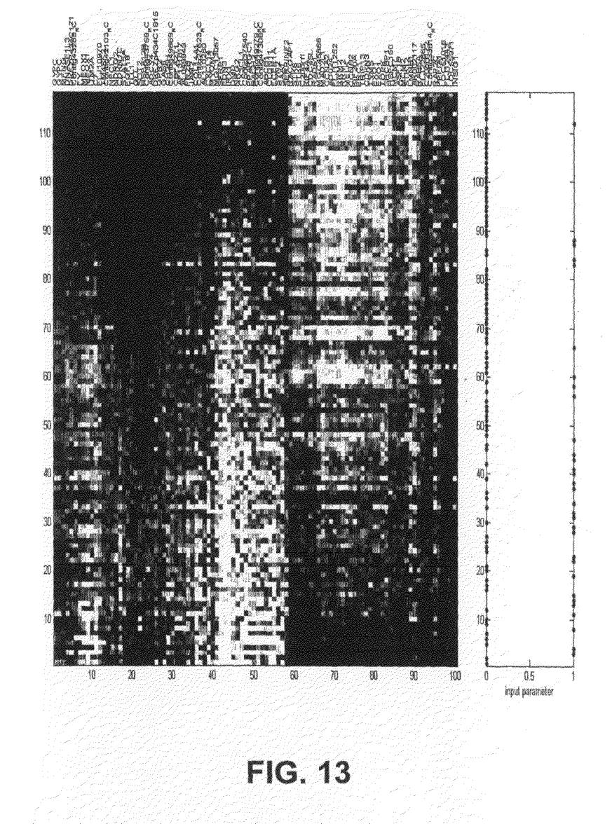

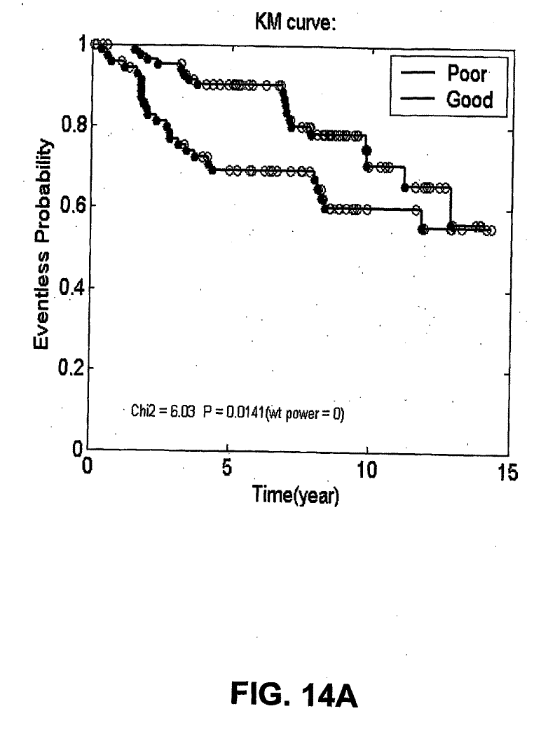

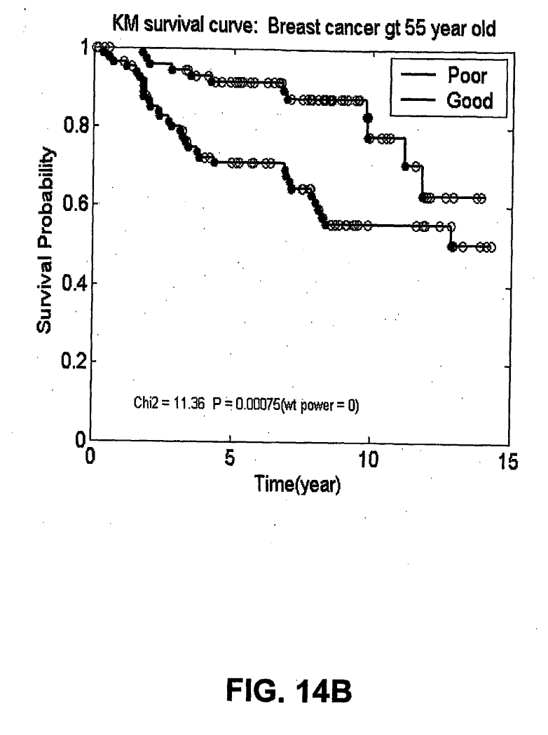

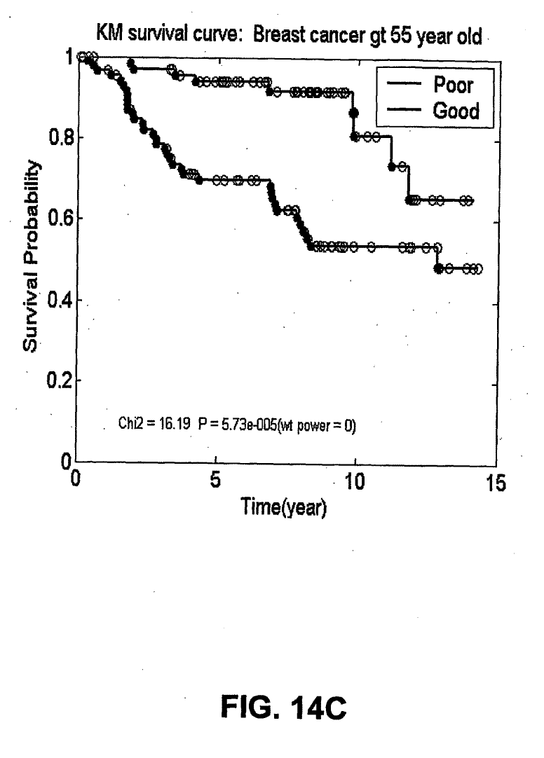

FIG. 11 shows the total error rate as a function of the number of discriminating genes for both methods. The error rates do not vary significantly with the number of genes in both cases. 200 reporter genes were therefore selected using the old algorithm (Tables 5 and 6), and 100 genes using the new algorithm (Tables 7 and 8). The discriminative patterns of these genes are shown in FIGS. 12 and 13, respectively. FIG. 14 compares the K-M plots for the 70 genes applied to the ER+ samples, the old algorithm and new algorithms. The results show that the old algorithm-derived 200-gene classifier improved significantly on the 70 gene classifier, and was, in turn, improved upon by the new algorithm derived 100-gene classifier (P-value of log-rank test improves from 1% for the 70-gene classifier, to 7.5E-4, and 5.7E-5 for the 200-marker and 100-marker classifiers, respectively). The odds ratio and average error rate (Table 10) also show the same trend. For example, the 5-year average error rate improved from 0.38 (70 gene) to 0.34 (old algorithm), to 0.27 (new algorithm).

TABLE 10
Comparison of three different classifier genesets in the prognosis
of samples from ER+ individuals age 55+.

| | | | average | average |
|---|---|---|---|---|
| | Overall | 5 year | Error Rate | Error Rate |
| | Odds Ratio | Odds Ratio | (overall) | (5 year) |
| 70 gene | 2.0 (0.8-4.8) | 2.9 (0.95-8.8) | 0.42 | 0.38 |
| Old method | 3.9 (1.7-9.3) | 3.9 (1.3-12.2) | 0.34 | 0.34 |
| new | 5.7 (2.3-14.1) | 7.1 (2.1-24.4) | 0.29 | 0.27 |
| method | | | | |

The gene-expression based classifiers for the purpose of prognosis suggests an application to clinical practices. The present classifier identifies a set of discriminating genes for the purposes of prognosis using gene expression profiles. The molecular classification of breast cancers on the basis of gene expression patterns can thus identify clinically significant subtype of cancers. The present study demonstrates that a global view of gene expression in breast cancer can bring clarity to previously difficult diagnostic categories. The precision of morphological diagnosis, even when assisted by immunohistochemstry for a few markers, was insufficient to identify diagnostic and prognostic subgroups.

### Example 5

### Biological Significance of Diagnostic Marker Genes

A search in the public domain was performed for functional annotations for several sets of marker genes for breast cancer prognosis in this age group, i.e., >55 years. Available gene descriptions and functional categories are listed in the corresponding table together with the gene list. See Tables 2, 4, 6, and 8. Of the total number of genes in each list, some percentage of genes is annotated. Interestingly, some key words such as “kinase” are involved in multiple genes among the annotated genes.

Kinases are important regulators of intracellular signal transduction pathways mediating cell proliferation, differentiation and apoptosis. Their activity is normally tightly controlled and regulated. Overexpression of certain kinases is known to be involved in oncogenesis, such as vascular endothelial growth factor receptors (VEGFR1 or FLT1), a tyrosine kinase that is an indicator of poor prognosis, which plays a very important role in tumor angiogenesis. Interestingly, vascular endothelial growth factor (VEGF), VEGFR's ligand, is also an indicator of poor prognosis, which means both ligand and receptor are co-upregulated in poor prognostic patients by an unknown mechanism. Given the total number of genes annotated among all 24,479 genes represented on the microarrays, an estimate the over-representation of the key word “kinase” in the list of genes of interest, and of the p-value of the number of “kinase” genes in the list, was made.

Cancer is characterized by deregulated cell proliferation. On the simplest level, this requires division of the cell, or mitosis. By keyword searching, “cell division” or “mitosis” was found to be included in 7 genes respectively in the 72 annotated entries from 156 genes indicating poor prognosis, and in zero genes in the 28 annotated genes from 75 genes that are indicators of good prognosis. Of 24,479 genes represented on the microarrays, there are 7,586 genes with annotations to date. “Cell division” is found in 62 gene annotations, and “mitosis” is found in 37 genes annotations. Given these statistics, the p-value that seven “cell division” or “mitosis” related genes in the group that are indicators of poor prognosis was estimated to be very significant (p-value=3.5×10⁻⁵). In comparison, the fact that no “cell division” or “mitosis” genes were found in the group of genes that are indicators of good prognosis was not found to be significant (p-value=0.69).

Cyclins, the regulatory subunits of cyclin-dependent kinases, control cell division or mitosis through key check-points within the cell cycle. Dysregulated expression and function of cyclins can lead to loss of normal growth control and cause uncontrolled expansion and invasion. Cyclin B2 and E2 were found to be overexpressed in poor prognostic patients.

Perspectives

The utility of classification by gene expression profiles is not limited to diagnosis and prognosis. Two developments may be anticipated in the near future as gene expression profiling becomes more widely used in medicine. First development would be identification of gene sets as predictors for prognosis of different cancer patients. It is expected that patient outcomes or response to therapy may be predicted by the overall expression pattern and/or the behavior of a set of specific marker genes. Identification of such markers is important beyond its diagnostic and prognostic potential, because in some cases a marker gene will itself contribute to tumor physiology. As microarray technology improves and becomes more widely available, expression analysis of a large variety of clinical samples will likely be employed to identify markers or patterns for diagnostic and prognostic purposes. If microarrays can then be manufactured at sufficiently low cost and reproducibility issues relating to sample purity and signal amplification convincingly resolved, expression profiles could become a standard molecular diagnostic and prognostic test. This new test could have substantially high specificity and sensitivity in situations where classical histo- or immunopathological approaches are unsatisfactory. The other development would be the discovery of candidate targets for therapy. It is conceivable that detailed studies of marker genes could help shed light into the underlying biological basis of different cancers and therefore could help identify corresponding therapeutic targets.

### 7. REFERENCES

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A computer-implemented method for determining a prognosis of an individual having breast cancer, comprising:
classifying, on a computer, said individual as having a good prognosis or a poor prognosis based on an expression profile comprising measurements of expression levels of a plurality of genes in a cell sample taken from the individual, said plurality of genes comprising 10 different genes for which markers are listed in any one or more of Tables 1, 3, 5 and 7 (SEQ ID NOS:1-387), wherein a good prognosis predicts no reoccurrence or metastasis within a predetermined period after initial diagnosis, and wherein a poor prognosis predicts reoccurrence or metastasis within said predetermined period after initial diagnosis.

2. The method of claim 1, wherein said plurality of genes comprises 20 different genes for which markers are listed in any one or more of Tables 1, 3, 5 and 7 (SEQ ID NOS:1-387).

3. The method of claim 1, wherein said plurality of genes comprises 50 different genes for which markers are listed in any one or more of Tables 1, 3, 5 and 7 (SEQ ID NOS:1-387).

4. The method of claim 1, wherein said plurality of genes comprises each of the genes for which markers are listed in Table 1.

5. The method of claim 1, wherein said plurality of genes comprises each of the genes for which markers are listed in Table 3.

6. The method of claim 1, wherein said individual is identified as ER+ (estrogen receptor positive), and said plurality of genes comprises 10 of the genes for which markers are listed in Table 5.

7. The method of claim 1, wherein said individual is identified as ER+ (estrogen receptor positive), and said plurality of genes comprises 50 of the genes for which markers are listed in Table 5.

8. The method of claim 1, wherein said individual is identified as ER+ (estrogen receptor positive), and said plurality of genes comprises each of the genes for which markers are listed in Table 5.

9. The method of claim 1, wherein said individual is identified as ER+ (estrogen receptor positive), and said plurality of genes comprises 10 of the genes for which markers are listed in Table 7.

10. The method of claim 1, wherein said individual is identified as ER+ (estrogen receptor positive), and said plurality of genes comprises 50 of the genes for which markers are listed in Table 7.

11. The method of claim 1, wherein said individual is identified as ER+ (estrogen receptor positive), and said plurality of genes comprises each of the genes for which markers are listed in Table 7.

12. The method of claim 1, wherein said classifying is carried out by a method comprising:
(a) comparing said expression profile to a good prognosis template comprising measurements of expression levels of said plurality of genes representative of expression levels of said plurality of genes in a plurality of good prognosis patients and/or to a poor prognosis template comprising measurements of expression levels of said plurality of genes representative of expression levels of said plurality of genes in a plurality of poor prognosis patients; and
(b) classifying said individual as having a good prognosis if said expression profile has a high similarity to said good prognosis template and/or has a low similarity to said poor prognosis template, or classifying said individual as having a poor prognosis if said expression profile has a low similarity to said good prognosis template and/or a high similarity to said poor prognosis template, wherein a high similarity corresponds to a degree of similarity above a predetermined threshold, and wherein a low similarity corresponds to a degree of similarity no greater than said predetermined threshold.

13. The method of claim 12, wherein the respective measurement of expression level of each gene in said plurality of genes in said good prognosis template or said poor prognosis template is an average of measured values of the expression levels of said gene in said plurality of good prognosis patients or in said plurality of poor prognosis patients, respectively.

14. The method of claim 13, wherein said average is an error-weighted average.

15. The method of claim 12, wherein said measurement of expression level of each gene in said expression profile is a differential expression level of said gene in said cell sample versus said gene in a first reference pool, represented as a log ratio; wherein the respective measurement of expression level of each gene in said plurality of genes in said good prognosis template is a differential expression level of said gene in said plurality of good prognosis patients versus said gene in a second reference pool, represented as a log ratio; and wherein the respective measurement of expression level of each gene in said plurality of genes in said poor prognosis template is a differential expression level of said gene in said plurality of poor prognosis patients versus said gene in a third reference pool, represented as a log ratio.

16. The method of claim 15, wherein the respective log ratio for each gene in said plurality of genes in said good prognosis template or said poor prognosis template is an average of the log ratios for said gene in said plurality of good prognosis patients or in said plurality of poor prognosis patients, respectively.

17. The method of claim 16, wherein said average is an error-weighted log ratio average.

18. The method of claim 12, said method comprising
(a) comparing said expression profile to a good prognosis template comprising measurements of expression levels of said plurality of genes representative of expression levels of said plurality of genes in a plurality of good prognosis patients; and
(b) classifying said individual as having a good prognosis if said expression profile has a high similarity to said good prognosis template, or classifying said individual as having a poor prognosis if said expression profile has a low similarity to said good prognosis template, wherein said similarity to said good prognosis template is represented by a first correlation coefficient between said expression profile and said good prognosis template, wherein said expression profile is said to have a high similarity to said good prognosis template if said first correlation coefficient between said expression profile and said good prognosis template is above a first threshold, and is said to have a low similarity to said good prognosis template if said first correlation coefficient between said expression profile and said good prognosis template is not above said first threshold.

19. The method of claim 18, wherein the respective measurement of expression level of each gene in said plurality of genes in said good prognosis template is an average of measured values of the expression levels of said gene in said plurality of good prognosis patients.

20. The method of claim 19, wherein said average is an error-weighted average.

21. The method of claim 18, wherein said measurement of expression level of each gene in said expression profile is a differential expression level of said gene in said cell sample versus said gene in a first reference pool, represented as a log ratio, and wherein the respective measurement of expression level of each gene in said plurality of genes in said good prognosis template is a differential expression level of said gene in said plurality of good prognosis patients versus said gene in a second reference pool, represented as a log ratio.

22. The method of claim 21, wherein the respective log ratio for each gene in said plurality of genes in said good prognosis template is an average of the log ratios for said gene in said plurality of good prognosis patients.

23. The method of claim 22, wherein said average is an error-weighted log ratio average.

24. The method of claim 18, wherein said first correlation coefficient between said expression profile and said good prognosis template is calculated according to the equation
P₁=(\vec{z}ᵢ·\vec{y})/(∥\vec{z}₁∥·∥\vec{y}∥)
wherein \vec{y} represents said expression profile, \vec{z}₁ represents said good prognosis template, and P₁ represents said first correlation coefficient between said expression profile and said good prognosis template.

25. The method of claim 1, wherein said classifying is carried out by a method comprising
(a) comparing said expression profile to a good prognosis template comprising measurements of expression levels of said plurality of genes representative of expression levels of said plurality of genes in a plurality of good prognosis patients and to a poor prognosis template comprising measurements of expression levels of said plurality of genes representative of expression levels of said plurality of genes in a plurality of poor prognosis patients; and
(b) classifying said individual as having a good prognosis if said expression profile has a higher similarity to said good prognosis template than to said poor prognosis template, or as having a poor prognosis if said expression profile has a higher similarity to said poor prognosis template than to said good prognosis template.

26. The method of claim 25, wherein the respective measurement of expression level of each gene in said plurality of genes in said good prognosis template and said poor prognosis template is an average of measured values of the expression levels of said gene in said plurality of good prognosis patients or in said plurality of poor prognosis patients, respectively.

27. The method of claim 26, wherein said average is an error-weighted average.

28. The method of claim 25, wherein said measurement of expression level of each gene in said expression profile is a differential expression level of said gene in said cell sample versus said gene in a first reference pool, represented as a log ratio; wherein the respective measurement of expression level of each gene in said plurality of genes in said good prognosis template is a differential expression level of said gene in said plurality of good prognosis patients versus said gene in a second reference pool, represented as a log ratio; and wherein the respective measurement of expression level of each gene in said plurality of genes in said poor prognosis template is a differential expression level of said gene in said plurality of poor prognosis patients versus said gene in a third reference pool, represented as a log ratio.

29. The method of claim 28, wherein the respective log ratio for each gene in said plurality of genes in said good prognosis template and said poor prognosis template is an average of the log ratios for said gene in said plurality of good prognosis patients or in said plurality of poor prognosis patients, respectively.

30. The method of claim 29, wherein said average is an error-weighted log ratio average.

31. The method of claim 25, wherein said similarity to said good prognosis template is represented by a first correlation coefficient between said expression profile and said good prognosis template, wherein said similarity to said poor prognosis template is represented by a second correlation coefficient between said expression profile and said poor prognosis template, and wherein said expression profile is said to have a higher similarity to said good prognosis template than to said poor prognosis template if said first correlation coefficient between said expression profile and said good prognosis template is greater than said second correlation coefficient between said expression profile and said good prognosis template.

32. The method of claim 31, wherein said first and second correlation coefficients between said expression profile and said good prognosis template and said poor prognosis template, respectively, are respectively calculated according to the equation
Pᵢ=(\vec{z}ᵢ·\vec{y})/(∥\vec{z}ᵢ∥·∥\vec{y}∥)
where i=1 and 2, wherein \vec{y} represents said expression profile, \vec{z}₁ represents said good prognosis template, and \vec{z}₂ represents said poor prognosis template, wherein P₁ represents said first correlation coefficient between said expression profile and said good prognosis template, and P₂ represents said second correlation coefficient between said expression profile and said poor prognosis template.

33. A method for determining a prognosis of an individual having breast cancer, comprising:
classifying said individual as having a good prognosis or a poor prognosis based on an expression profile comprising measurements of expression levels of a plurality of genes in a cell sample taken from the individual, said plurality of genes comprising 10 different genes for which markers are listed in any one or more of Tables 1, 3, 5 and 7 (SEQ ID NOS:1-387), wherein a good prognosis predicts no reoccurrence or metastasis within a predetermined period after initial diagnosis, and wherein a poor prognosis predicts reoccurrence or metastasis within said predetermined period after initial diagnosis, wherein said classifying comprises the steps of:
(a) generating a good prognosis template by hybridization of nucleic acids derived from a plurality of good prognosis patients against nucleic acids derived from a pool of tumors from a plurality of patients having breast cancer;
(b) generating a poor prognosis template by hybridization of nucleic acids derived from a plurality of poor prognosis patients against nucleic acids derived from said pool of tumors from said plurality of patients;
(c) generating said expression profile by hybridizing nucleic acids derived from said cell sample taken from said individual against said pool; and
(d) determining the similarity of said expression profile to the good prognosis template and to the poor prognosis template, wherein if said expression profile is more similar to the good prognosis template, the individual is classified as having a good prognosis, and if said expression profile is more similar to the poor prognosis template, the individual is classified as having a poor prognosis.

34. A computer-implemented method for assigning a person to one of a plurality of categories in a clinical trial, comprising.
(a) classifying, on a computer, said individual as having a good prognosis or a poor prognosis based on an expression profile comprising measurements of expression levels of a plurality of genes in a cell sample taken from the individual, said plurality of genes comprising 10 different genes for which markers are listed in any one or more of Tables 1, 3, and 7 (SEQ ID NOS:1-387), wherein a good prognosis predicts no reoccurrence or metastasis within a predetermined period after initial diagnosis, and wherein a poor prognosis predicts reoccurrence or metastasis within said predetermined period after initial diagnosis; and
(b) assigning said person to one category in a clinical trial if said person is classified as having a good prognosis, and a different category if that person is classified as having a poor prognosis.

35. The method of claim 18, said method further comprising classifying said individual as having a very good prognosis if said first correlation coefficient between said expression profile and said good prognosis template is above a second threshold, said second threshold is greater than said first threshold, or an intermediate prognosis if said first correlation coefficient between said expression profile and said good prognosis template is above a first threshold but not above said second threshold is greater than said first threshold.

36. The method of claim 1, wherein said measurement of expression level of each gene in said expression profile is a differential expression level of said gene in said cell sample versus said gene in a reference pool.

37. The method of claim 36, wherein said differential expression level is represented as a log ratio.

38. The method of any one of claim 1, wherein said reference pool is derived from a normal breast cell line or from a breast cancer cell line or from tumors from sporadic breast cancer patients.

39. A method for determining a prognosis of an individual having breast cancer, comprising:
(a) determining an expression profile by measuring expression levels of a plurality of genes in a cell sample taken from said individual, said plurality of genes comprising 10 different genes for which markers are listed in any one or more of Tables 1, 3, 5 and 7 (SEQ ID NOS:1-387); and
(b) classifying said individual as having a good prognosis or a poor prognosis based on said expression profile, wherein a good prognosis predicts no reoccurrence or metastasis within a predetermined period after initial diagnosis, and wherein a poor prognosis predicts reoccurrence or metastasis within said predetermined period after initial diagnosis.

40. The method of claim 1, wherein said individual is 55 years of age or older.

41. The method of claim 1, wherein said predetermined period is 5 years.

42-58. (canceled)
